(19) United States
(12) Patent Application Publication
(10) Pub. No.: US 2010/0028469 A1
(43) Pub. Date: Feb. 4, 2010

(54) Extracts of Cranberry and Methods of Using Thereof

(75) Inventors: Randall S. Alberte, Estero, FL (US); William P. Roschek, JR., Naples, FL (US); Dan Li, Singapore (SG)

Correspondence Address: FOLEY HOAG, LLP;PATENT GROUP, WORLD TRADE CENTER WEST, 155 SEAPORT BLVD, BOSTON, MA, 02110, US

(73) Assignee: HerbalScience Group, LLC, Naples, FL (US)

(21) Appl. No.: 12/478,431

(22) Filed: Jun. 4, 2009

Related U.S. Application Data
(60) Provisional application No. 61/058,911, filed on Jun. 4, 2008.
(60) Provisional application No. 61/101,513, filed on Sep. 30, 2008.

(51) Int. Cl. A61K 36/45; A61K 31/195; A61K 31/70; A61K 31/415; A61P 13/00

(52) U.S. Cl. 424/732; 514/561; 514/55; 514/393

(57) ABSTRACT

The present disclosure relates in part to extracts of cranberry (Vaccinium macrocarpon) comprising an enriched amount of certain compounds having anti-infective activity, e.g. antibacterial and/or antifungal activity, e.g. activity against C. albicans. Another aspect of the disclosure relates to combined cranberry and cinnamon extracts. In certain embodiments, these combined extracts have been optimized to control urinary tract infections caused by E. coli, S. aureus and C. albicans. Certain embodiments of the extract are enriched in bioactive compounds that have been shown to inhibit C. albicans adhesion and/or biofilm formation and its growth in vitro. In another aspect of the disclosure, the extracts are enriched in bioactives derived from cranberry and cinnamon that have been shown to inhibit the attachment and the growth of common urinary tract pathogens like E. coli, S. aureus and C. albicans.

### RELATED APPLICATIONS

This application claims the benefit of priority to U.S. Provisional Application Nos. 61/101,513, filed on Sep. 30, 2008, and 61/058,911, filed on Jun. 4, 2008, the contents of which are hereby incorporated in their entirety.

### BACKGROUND OF THE INVENTION

Urinary tract infections (UTI) have been a pervasive health care problem. It is well established that UTI are caused by microbial infections, perhaps most notably a Gram negative prokaryote, Escherichia coli, and more recently the Gram positive bacterium, Staphylococcus aureus, and a single-celled eukaryote, Candida albicans. The main characteristic that allows these microorganisms to be successful pathogens and survive in the hostile nosocomial environment is their ability to form biofilms on surfaces, thus preventing and counteracting the action of antibiotics and commonly used disinfectants.

The yeast, C. albicans, can cause pervasive fungal infections for many women. Nearly 75% of all women will experience a yeast infection at least one time in their life, and half of these women will experience recurrent infections (C. A. Rodgers and A. J. Beardall, 1999. Recurrent vulvovaginal candidiasis: why does it occur? International Journal of STD & AIDS. 10:435-439). Candida. albicans is prevalent infectious agent because of its biofilm lifestyle (J. W. Costerton, P. S. Stewart and E. P. Greenberg, 1999. Bacterial biofilms: a common cause of persistent infections. Science. 284:1318-1322; R. M. Donlan, 2002. Biofilms: microbial life on surfaces. Emerging Infectious Diseases. 8:881-890; M. A. Jabra-Rizk, W. A. Falkler and T. F. Meiller, 2004. Fungal biofilms and drug resistance. Emerging Infectious Diseases. 10: 14-19).

The biofilm formation process that C. albicans utilizes encompasses multiple steps. The first step is the production of a biological ‘glue’, then adhesion of C. albicans to a surface (manmade or natural), followed by the proliferation of C. albicans into a biofilm that initiates an inflammatory response and, in some cases, cellular invasion and entry into the bloodstream (M. A. Jabra-Rizk, W. A. Falkler and T. F. Meiller, 2004. Fungal biofilms and drug resistance. Emerging Infectious Diseases. 10:14-19; A. Escher and W. Characklis, 1990. Modeling the initial events in biofilm accumulation. BioFilms. 445-486). This latter step results in a severe toxic response termed candidiasis. Mortality is associated with candidiasis in greater than 25% of all incidences, and candidaemia rates have been increasing rapidly to the point that they are now the fourth-most-common cause of bloodstream infections in the U.S. (M. B. Edmond, S. E. Wallace, D. K. McClish, M. A. Pfaller, R. N. Jones and R. P. Wenzel, 1999. Nosocomial bloodstream infections in United States hospitals: a three-year analysis. Clinical Infectious Diseases. 29:239-244; D. A. Enoch, H. A. Ludlam and N. M. Brown, 2006. Invasive fungal infections: a review of epidemiology and management options. Journal of Medical Microbiology. 55:809-818). For these reasons, prevention of C. albicans adhesion, the first step in the infection process, is a fundamental, important, and powerful means to control and treat yeast infections (B. Barrett, D. Kiefer and D. Rabago, 1999. Assessing the risks and benefits of herbal medicine: an overview of scientific evidence. Alternative Therapies in Health and Medicine. 5:40-49; R. S. Alberte and R. D. Smith, 2006. Generation of combinatorial synthetic libraries and screening for novel proadhesins and antiadhesions; R. S. Alberte, R. D. Smith and R. C. Zimmerman, 2007. Safe and effective biofilm inhibitory compounds and health related uses thereof, L. Cegelski, G. R. Marshall, G. R. Eldridge and S. J. Hultgren, 2008. The biology and future prospects of antivirulence therapies. Nature Reviews: Microbiology. 6:17-27; M. G. Netea, G. D. Brown, B. J. Kullberg and N. A. Gow, 2008. An integrated model of the recognition of Candida albicans by the innate immune system. Nature Reviews: Microbiology. 6:67-78).

The worldwide anti-fungal market is valued over $1 billion, of which feminine hygiene products represent about one third. This market is growing at about 5.1% a year and the bulk is in OTC products. Yeast infections caused by C. albicans in women are most often recurrent and afflict over 15 million in the U.S. alone. Though most treatments are topical creams or lotions, there are several oral products. Current antifungal products for yeast infections when taken orally have significant side-effects (D. A. Enoch, H. A. Ludlam and N. M. Brown, 2006. Invasive fungal infections: a review of epidemiology and management options. Journal of Medical Microbiology. 55:809-818). Resistance generation in C. albicans is high, particularly from OTC azole-based products (M. A. Jabra-Rizk, W. A. Falkler and T. F. Meiller, 2004. Fungal biofilms and drug resistance. Emerging Infectious Diseases. 10:14-19; B. Mathema, E. Cross, E. Dun, S. Park, J. Bedell, B. Slade, M. Williams, L. Riley, V. Chaturvedi and D. S. Perlin, 2001. Prevalence of vaginal colonization by drug-resistant Candida species in college-age women with previous exposure to over-the-counter azole antifungals. Clinical Infectious Diseases. 33:E23-E27), the most common anti-yeast agents, and multi-drug resistant strains are becoming increasingly widespread (D. A. Enoch, H. A. Ludlam and N. M. Brown, 2006. Invasive fungal infections: a review of epidemiology and management options. Journal of Medical Microbiology. 55:809-818; D. Sanglard and F. C. Odds, 2002. Resistance of Candida species to antifungal agents: molecular mechanisms and clinical consequences. Lancet Infectious Diseases. 2:73-85; S. MacPherson, B. Akache, S. Weber, X. De Deken, M. Raymond and B. Turcotte, 2005. Candida albicans zinc cluster protein Upc2p confers resistance to antifungal drugs and is an activator of ergosterol biosynthetic genes. Antimicrobial Agents and Chemotherapy. 49:1745-1752). Therefore, there is not only a need for new antifungal treatments for yeast infections that can minimize side-effects, but also those that address new therapeutic targets to treat multi-drug resistant strains.

Pathogen biofilms are particularly difficult to treat (J. W. Costerton, P. S. Stewart and E. P. Greenberg, 1999. Bacterial biofilms: a common cause of persistent infections. Science. 284:1318-1322; R. M. Donlan, 2002. Biofilms: microbial life on surfaces. Emerging Infectious Diseases. 8:881-890) and Candida biofilms are no exception (M. A. Jabra-Rizk, W. A. Falkler and T. F. Meiller, 2004. Fungal biofilms and drug resistance. Emerging Infectious Diseases. 10: 14-19). A biofilm lifestyle requires that pathogens attach themselves to surfaces, a process mediated by the production of biological glues that also function in host recognition (R. M. Donlan, 2002. Biofilms: microbial life on surfaces. Emerging Infectious Diseases. 8:881-890; L. Cegelski, G. R. Marshall, G. R. Eldridge and S. J. Hultgren, 2008. The biology and future prospects of antivirulence therapies. Nature Reviews: Microbiology. 6:17-27; M. G. Netea, G. D. Brown, B. J. Kullberg and N. A. Gow, 2008. An integrated model of the recognition of Candida albicans by the innate immune system. Nature Reviews: Microbiology. 6:67-78). Biofilms offer a physical environment that protects pathogens from most known anti-microbial agents (whether antibiotics or anti-fungals), that target intracellular metabolic functions (J. W. Costerton, P. S. Stewart and E. P. Greenberg, 1999. Bacterial biofilms: a common cause of persistent infections. Science. 284:1318-1322). Though the reasons for this protection is not fully understood (R. M. Donlan, 2002. Biofilms: microbial life on surfaces. Emerging Infectious Diseases. 8:881-890; M. A. Jabra-Rizk, W. A. Falkler and T. F. Meiller, 2004. Fungal biofilms and drug resistance. Emerging Infectious Diseases. 10:14-19), the extensive extracellular matrix that is characteristic of biofilms is a major contributor (D. G. Allison, 2003. The biofilm matrix. Biofouling. 19:139-150). Recent work suggests that agents that interfere with biofilm formation and stability by acting on components of the extracellular matrix can dramatically enhance the effectiveness of antibiotics on bacterial biofilms (M. W. Mittelman, N. Allan, M. E. Olson, D. Vaughan and R. S. Alberte, 2008. Enhancement of in vitro antibiotic efficacy against Staphylococcus ssp. biofilms with a novel adhesion inhibitor. Antimicrobial Agents & Chemotherapy. In Preparation). Though only recognized in the last two decades (J. W. Costerton, P. S. Stewart and E. P. Greenberg, 1999. Bacterial biofilms: a common cause of persistent infections. Science. 284:1318-1322; N. Sharon and I. Ofek, 2002. Fighting infectious diseases with inhibitors of microbial adhesion to host tissues. Critical Reviews in Food Science and Nutrition. 42:267-272), the development of new anti-microbials that target pathogen adhesion/recognition, the first step in infection and a key virulence factor, is viewed as key to future anti-virulence therapies. In fact, (L. Cegelski, G. R. Marshall, G. R. Eldridge and S. J. Hultgren, 2008. The biology and future prospects of antivirulence therapies. Nature Reviews: Microbiology. 6:17-27) have stated that targeting virulence represents a new paradigm to empower the clinician to prevent and treat infectious disease.

Biofilm formation is a process that encompasses multiple steps; however, the first critical stage is the adhesion of the microbes to a surface in order to serve as an anchor to other microorganism of the same or a different species (S. M. Opal, 2007. Communal living by bacteria and the pathogenesis of urinary tract infections. PLoS Medicine. 4:e349; D. A. Rosen, T. M. Hooton, W. E. Stamm, P. A. Humphrey and S. J. Hultgren, 2007. Detection of intracellular bacterial communities in human urinary tract infection. PLoS Medicine. 4:e329). As a result, prevention of adhesion of these microorganisms would be fundamental for the treatment of UTI's.

Cranberry was introduced to European settlers by Native Americans who used these berries for the treatment of kidney stones and urinary tract health problems (B. Barrett, D. Kiefer and D. Rabago, 1999. Assessing the risks and benefits of herbal medicine: an overview of scientific evidence. Alternative Therapies in Health and Medicine. 5:40-49). Since that time, cranberry has been used to treat a number of ailments such as urinary tract infections, scurvy, stomach ailments, vomiting, and weight loss by a large part of the U.S. population (B. Barrett, D. Kiefer and D. Rabago, 1999. Assessing the risks and benefits of herbal medicine: an overview of scientific evidence. Alternative Therapies in Health and Medicine. 5:40-49; D. V. Moen, 1962. Observations on the effectiveness of cranberry juice in urinary infections. Wisconsin Medical Journal. 61:282-283). There are a number of cranberry extracts on the market, and cranberry juice is a common and popular beverage alone or in combination with other juices. In addition, there is public recognition of the health benefits of cranberry-based products (R. G. Jepson and J. C. Craig, 2008. Cranberries for preventing urinary tract infections. Cochrane Database of Systematic Reviews (Online). CD001321).

The mode of action of cranberry against UTI is unclear and has been attributed to several potential mechanisms. One mechanism is the acidification of urine, due to bacteria preferring less acidic conditions for growth (D. V. Moen, 1962. Observations on the effectiveness of cranberry juice in urinary infections. Wisconsin Medical Journal. 61:282-283; F. C. Lowe and E. Fagelman, 2001. Cranberry juice and urinary tract infections: what is the evidence? Urology. 57:407-413; A. B. Howell, N. Vorsa, A. Der Marderosian and L. Y. Foo, 1998. Inhibition of the adherence of P-fimbriated Escherichia coli to uroepithelial-cell surfaces by proanthocyanidin extracts from cranberries. New England Journal of Medicine. 339:1085-1086) although the pH change of urine after drinking cranberry is minimal. Also, the UTI interference has been attributed to the hippuric acid content, which is a metabolic product of benzoic acid, a known antimicrobial agent. More recent research has focused on the flavonoid content of cranberries, specifically cranberry proanthocyanidins (PACs). These PACs inhibit fimbriae binding of uropathogenic E. coli to host cells in the urinary tract and function as anti-adhesions by binding to the host cells, preventing the fimbrae of E. coli to adhere, and thus form a biofilm (A. B. Howell, N. Vorsa, A. Der Marderosian and L. Y. Foo, 1998. Inhibition of the adherence of P-fimbriated Escherichia coli to uroepithelial-cell surfaces by proanthocyanidin extracts from cranberries. New England Journal of Medicine. 339:1085-1086; A. B. Howell, 2007. Bioactive compounds in cranberries and their role in prevention of urinary tract infections. Molecular Nutrition & Food Research. 51:732-737).

A very common treatment for bacterial and fungal infections is the use of cinnamon (Cinnamomum cassia) extracts. The antimicrobial action of cinnamon can be partly attributed to the presence of cinnamaldehyde, eugenol, borneol, linool, and thymol, mainly antibacterial, and o-methoxycinnamaldehyde, mainly antifungal.

Although there is a large literature on the role of cranberry phytonutrients in preventing or mitigating urinary tract infections (UTIs) (J. P. Lavigne, G. Bourg, C. Combescure, H. Botto and A. Sotto, 2008. In-vitro and in-vivo evidence of dose-dependent decrease of uropathogenic Escherichia coli virulence after consumption of commercial Vaccinium macrocarpon (cranberry) capsules. Clinical Microbiology and Infection. 14:350-355; I. Ofek, J. Goldhar and N. Sharon, 1996. Anti-Escherichia coli adhesion activity of cranberry and blueberry juices. Advances in Experimental Medicine and Biology. 408:179-183; I. Ofek, J. Goldhar, D. Zafriri, H. L is, R. Adar and N. Sharon, 1991. Anti-Escherichia coli adhesion activity of cranberry and blueberry juices. New England Journal of Medicine. 324:1599), and particularly the Gram negative uropathogenic bacterium E. coli, the most common cause of UTIs, most of the reports on cranberry fruit for the control of yeast infections are anecdotal. Yeasts, though microbes like bacteria, are eukaryotic, therefore traditional antibiotics have no efficacy against them. Most anti-fungals generate significant side effects, and these are realized in 50-90% of patients taking oral anti-fungal treatments. For this reason, vaginal Candida infections are most often treated with OTC topical anti-fungals that minimize side effects, but sacrifice efficacy and lead to the generation of resistant yeast strains. Therefore, there is a need for new treatments for yeast infections that are safe and effective, and that can minimize the risk of recurrent infections and candidiasis. There is also a need for new treatments for urinary tract infections.

### SUMMARY OF THE INVENTION

One aspect of the invention relates to extracts of cranberry (Vaccinium macrocarpon) comprising an enriched amount of certain compounds having anti-infective activity, e.g. antibacterial and/or antifungal activity, e.g. activity against C. albicans. In certain embodiments, the extract has been optimized for use for control of yeast (C. albicans) infections for feminine hygiene. Another aspect of the invention relates to combined cranberry and cinnamon extracts. In certain embodiments, these combined extracts have been optimized to control urinary tract infections caused by E. coli, S. aureus and C. albicans. In certain embodiments, the extract possesses over 500 compounds detected by DART TOF-MS of which 94 were identified. Certain embodiments of the extract are enriched in bioactive compounds that have been shown to inhibit C. albicans adhesion and/or biofilm formation and its growth in vitro—two key anti-microbial properties that can control and mitigate yeast infections. In another aspect of the invention, the extracts are enriched in bioactives derived from cranberry and cinnamon that have been shown to inhibit the attachment and the growth of common urinary tract pathogens like E. coli, S. aureus and C. albicans. The inhibition of attachment, biofilm formation and growth of UTI pathogens will all block and/or mitigate urinary tract infections.

### BRIEF DESCRIPTION OF THE DRAWINGS

FIG. 1 depicts a DART TOF mass spectrum of cranberry Extract 1.

FIG. 2 depicts a DART TOF mass spectrum of cranberry Extract 2.

FIG. 3 depicts a DART TOF mass spectrum of cranberry Extract 3.

FIG. 4 depicts a DART TOF mass spectrum of cranberry Extract 4.

FIG. 5 depicts a DART TOF mass spectrum of cranberry Extract 5.

FIG. 6 depicts a DART TOF mass spectrum of cranberry Extract 6.

FIG. 7 depicts a DART-TOF mass spectrum of chemistries in Extract 6 bound to E. coli after being subjected to the direct binding assay.

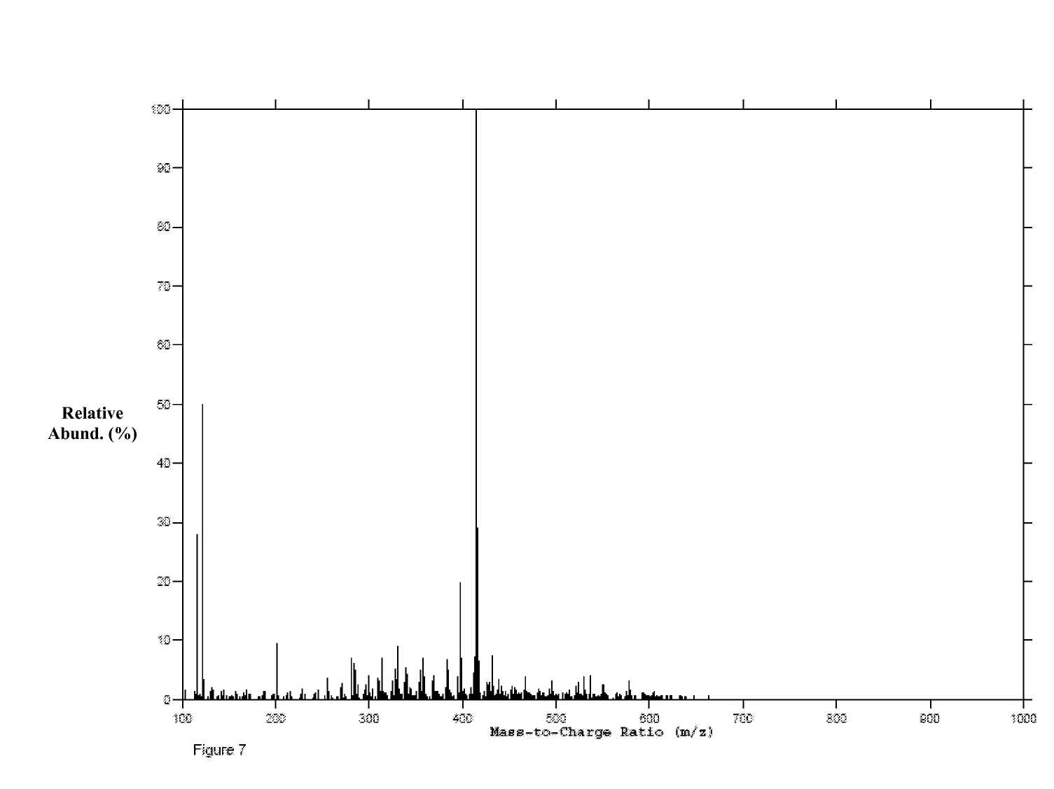

FIG. 8 depicts a DART-TOF mass spectrum of chemistries in Extract 6 bound to C. albicans after being subjected to the direct binding assay.

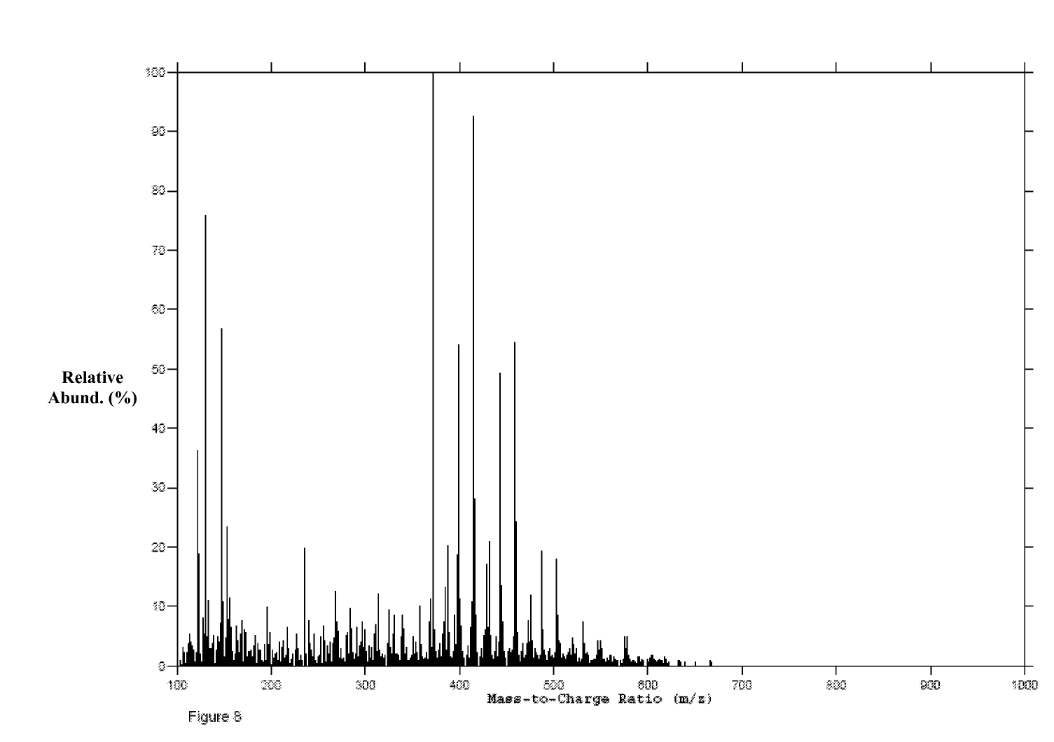

FIG. 9 depicts a DART-TOF spectrum of chemistries in Extract 6 bound to S. aureus (methicillin resistant; MRSA) after being subjected to the direct binding assay.

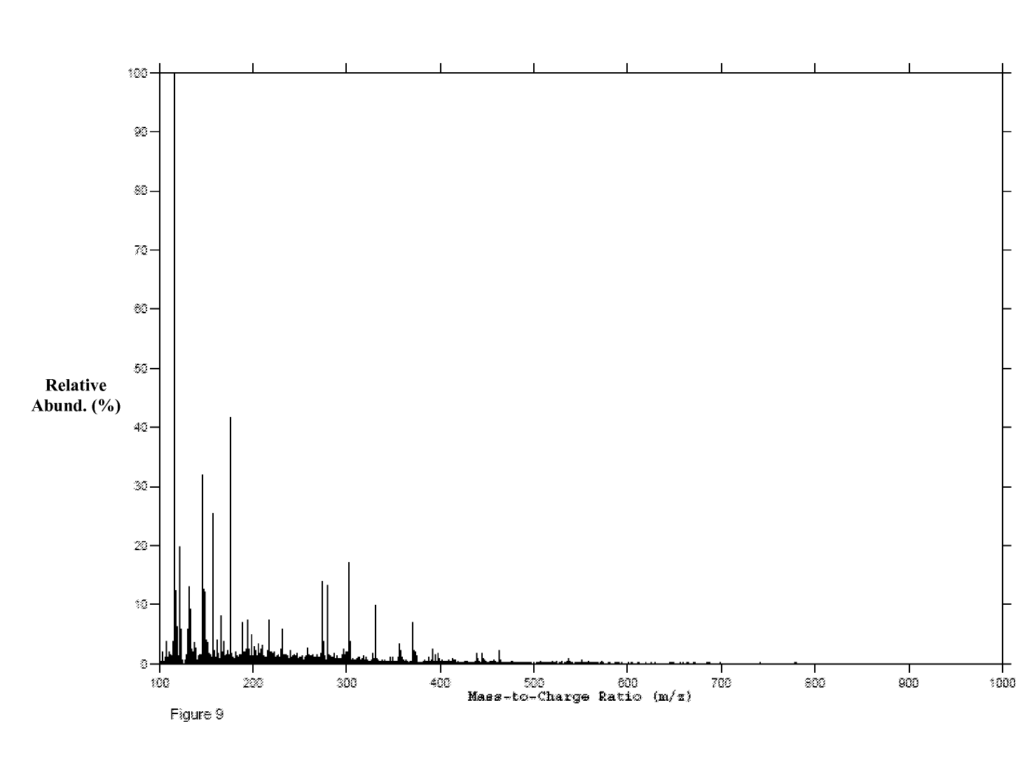

FIG. 10 depicts a pharmacokinetic profile of key bioactives of the cranberry extract that are bioavailable in serum as determined by DART TOF-MS.

FIG. 11 depicts a pharmacokinetic profile of key bioactives of the cranberry extract that are bioavailable in urine as determined by DART TOF-MS.

FIG. 12 depicts a pharmacokinetic profile of key bioactives of Extract 6 that are present in urine as determined by DART TOF-MS.

### DETAILED DESCRIPTION OF THE INVENTION

### Definitions

The term “effective amount” as used herein refers to the amount necessary to elicit the desired biological response. As will be appreciated by those of ordinary skill in this art, the effective amount of a composite or bioactive agent may vary depending on such factors as the desired biological endpoint, the bioactive agent to be delivered, the composition of the encapsulating matrix, the target tissue, etc.

As used herein, the term “extract” refers to a product prepared by extraction. The extract may be in the form of a solution in a solvent, or the extract may be a concentrate or essence which is free of, or substantially free of solvent. The term extract may be a single extract obtained from a particular extraction step or series of extraction steps or the extract also may be a combination of extracts obtained from separate extraction steps. For example, extract “a” may be obtained by extracting cranberry with alcohol in water, while extract “b” may be obtained by super critical carbon dioxide extraction of cranberry. Extracts a and b may then be combined to form extract “c”. Such combined extracts are thus also encompassed by the term “extract”.

As used herein, the term “fraction” means the extract comprising a specific group of chemical compounds characterized by certain physical, chemical properties or physical or chemical properties.

As used herein, the term “profile” refers to the ratios by percent mass weight of the chemical compounds within an extraction fraction or to the ratios of the percent mass weight of each of the chemical constituents in a final cranberry, cinnamon or combined cranberry and cinnamon extract.

As used herein, the term “purified” fraction or composition means a fraction or composition comprising a specific group of compounds characterized by certain physical-chemical properties or physical or chemical properties that are concentrated to greater than 50% of the fraction's or composition's chemical constituents. In other words, a purified fraction or composition comprises less than 50% chemical constituent compounds that are not characterized by certain desired physical-chemical properties or physical or chemical properties that define the fraction or composition.

The term “synergistic” is art recognized and refers to two or more components working together so that the total effect is greater than the sum of the components.

The term “treating” is art-recognized and refers to curing as well as ameliorating at least one symptom of any condition or disorder.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic (i.e., it is intended to diminish, ameliorate, or stabilize the existing unwanted condition or side effects thereof).

The term “preventing”, when used in relation to a condition, such as cancer, an infectious disease, or other medical disease or condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount. Prevention of an infection includes, for example, reducing the number of diagnoses of the infection in a treated population versus an untreated control population, and/or delaying the onset of symptoms of the infection in a treated population versus an untreated control population.

As used herein, the term “microbe” refers to a microscopic organism, usually invisible to the naked eye (e.g., bacteria, yeasts).

As used herein, the term “bacterium” refers to a prokaryotic class of unicellular (single or chains) organisms or microbes that lack an defined and organized nucleus and fall into two general classes Gram-positive and Gram negative based on the chemically staining properties of their cell wall.

As used herein, the term “urinary tract infection” or “UTI” refers to a bacterial infection that affects any part of the urinary tract. When bacteria get into the bladder or kidney and multiply in the urine, they cause a UTI. The most common type of UTI is a bladder infection which is also often called cystitis.

As used herein, the term “yeast infection” refers to a fungal infection (mycosis) of any of the Candida species, of which C. albicans is the most common. Candidiasis encompasses infections that range from superficial, such as oral thrush and vaginitis, to systemic and potentially life-threatening diseases. Candida infections of the latter category are also referred to as candidemia and are usually confined to severely immunocompromised persons, such as cancer, transplant, and AIDS patients.

As used herein, the term “adhesion” refers to the binding of a cell to a surface, extracellular matrix or another cell or a manmade material using cell adhesion molecules such as selecting, integrins, and cadherins or, more generally, adhesins.

As used herein, the term “biostatic” refers to molecules that inhibit growth and reproduction of bacteria without killing them.

As used herein, the term “biofilm” refers to a structured community of microorganisms encapsulated within a self-developed polymeric matrix and adherent to a living or inert surface. Biofilms are also often characterized by surface attachment, structural heterogeneity, genetic diversity, complex community interactions, and an extracellular matrix of polymeric substances. Single-celled organisms generally exhibit two distinct modes of behavior. The first is the familiar free floating, or planktonic, form in which single cells float or swim independently in some liquid medium. The second is an attached state in which cells are closely packed and firmly attached to each other and usually form a solid surface. A change in behavior is triggered by many factors, including quorum sensing, as well as other mechanisms that vary between species. When a cell switches modes, it undergoes a phenotypic shift in behavior in which large suites of genes are up- and down-regulated.

Extracts

One aspect of the invention relates to extracts of cranberry comprising an enriched amount of certain compounds having anti-infective activity, e.g., antibacterial and/or antifungal activity, e.g., activity against C. albicans. In certain embodiments, the extract has been optimized for use for control of yeast (C. albicans) infections for feminine hygiene. In certain embodiments, the extract possesses over 500 compounds detected by DART TOF-MS of which 94 were identified. Certain embodiments of the extract are enriched in bioactive compounds that have been shown to inhibit C. albicans adhesion and/or biofilm formation and its growth in vitro, representing two key anti-microbial properties that can control and mitigate yeast infections.

While not being bound by any particular theory, it is believed that the cranberry extracts of the present invention represent a ‘first-in-class’ product for yeast infections by blocking the first step in the infection process, through the binding of bioactive compounds to yeast surface domains involved in host recognition, adhesion and biofilm formation. C. albicans adhesins are mannose-rich extracellular polymers that fall into two classes, Als (Agglutinin-like Sequence) and Hwp1 proteins (S. A. Klotz, N. K. Gaur, D. F. Lake, V. Chan, J. Rauceo and P. N. Lipke, 2004. Degenerate peptide recognition by Candida albicans adhesins Als5p and Als1p. Infection and Immunity. 72:2029-2034; C. J. Nobile, J. E. Nett, D. R. Andes and A. P. Mitchell, 2006. Function of Candida albicans adhesin Hwp1 in biofilm formation. Eukaryotic Cell. 5:1604-1610; J. M. Rauceo, R. De Armond, H. Otoo, P. C. Kahn, S. A. Klotz, N. K. Gaur and P. N. Lipke, 2006. Threonine-rich repeats increase fibronectin binding in the Candida albicans adhesin Als5p. Eukaryotic Cell. 5:1664-1673). These mannose-rich glycoproteins dictate and control adhesion of C. albicans in vitro and in vivo, and bind in vivo, to a variety of receptors, including Toll-like Receptor 4 (TLR4), Mannan Receptors, DC-SIGN Receptors, and Dectin 1 Receptors which induce the inflammatory cascade associated with C. albicans infections (M. G. Netea, G. D. Brown, B. J. Kullberg and N. A. Gow, 2008. An integrated model of the recognition of Candida albicans by the innate immune system. Nature Reviews: Microbiology. 6:67-78).

Flavonoids and proanthocyanidins in the extracts bind to C. albicans and block the ability of the yeast to adhere to surfaces and form biofilms. Other novel synthetic chemistries have been described that function in a similar manner and are highly effective against a variety of bacterial and fungal species including C. albicans (R. S. Alberte and R. D. Smith, 2005. Generation of combinatorial synthetic libraries and screening for novel proadhesins and antiadhesins; R. S. Alberte, R. D. Smith and R. C. Zimmerman, 2006. Safe and effective biofilm inhibitory compounds and health related uses thereof.).

In addition, the extracts contain chemicals that inhibit the growth of C. albicans, thus providing two anti-fungal modes-of-action. Based on the in vitro activities described here, the cranberry extracts described herein address the key process involved in yeast infections and can promote feminine hygiene. Furthermore, the extracts can be delivered in a quick-dissolving lozenge that allows for sublingual and/oral cavity absorption.

In some embodiments, the invention relates to a cranberry extract comprising at least one compound selected from the group consisting of aminoevulinic acid, abscisic acid, S-petasine, fraxin, and schisandrol B. In certain embodiments, the extract comprises at least one of the aforementioned compounds in the following amounts: 0.5 to 10% by weight aminoevulinic acid, 0.5 to 10% by weight of abscisic acid, 0.01 to 5% by weight of S-petasine, 0.01 to 5% by weight of fraxin, and 0.01 to 5% by weight of schisandrol B.

In some embodiments, the extract comprises 0.01 to 5% by weight of schisandrol B.

In some embodiments, the aforementioned extracts comprise 0.01 to 5% by weight of fraxin. In other embodiments, the aforementioned extracts comprise 0.1 to 10% by weight of S-petasine. In other embodiments, the aforementioned extract comprises 0.5 to 10% by weight of abscisic acid. In further embodiments, any of the aforementioned extracts comprises 0.5 to 10% by weight aminoevulinic acid. In some embodiments, the cranberry extract comprises at least one compound selected from the group consisting of 0.5 to 5% by weight aminoevulinic acid, 0.5 to 5% by weight of abscisic acid, 0.01 to 2% by weight of S-petasine, 0.01 to 2% by weight of fraxin, and 0.05 to 3% by weight of schisandrol B.

In certain embodiments, the extract comprises at least one of the aforementioned compounds in the following amounts: 500 to 5000 μg aminoevulinic acid, 500 to 5000 μg abscisic acid, 10 to 1000 μg S-petasine, 5 to 1000 μg fraxin, or 10 to 1000 μg schisandrol B, per 100 mg of extract.

In other embodiments, the extract comprises cinnamaldehyde, 0.1 to 5% L-threonine by weight of the cinnamaldehyde, 1 to 10% aminoevulinic acid by weight of the cinnamaldehyde, 1 to 15% 4-hydroxybenzoic acid by weight of the cinnamaldehyde, 5 to 20% anethole/cuminaldehyde by weight of the cinnamaldehyde, 1 to 10% chitosan by weight of the cinnamaldehyde, 10 to 25% α-phenylindol by weight of the cinnamaldehyde, 5 to 20% biotin by weight of the cinnamaldehyde, 10 to 25% abscisic acid by weight of the cinnamaldehyde, 20 to 50% vestitol by weight of the cinnamaldehyde, 5 to 20% S-petasine by weight of the cinnamaldehyde, 0.1 to 5% fraxin by weight of the cinnamaldehyde, and 1 to 15% Schisandrol B by weight of the cinnamaldehyde.

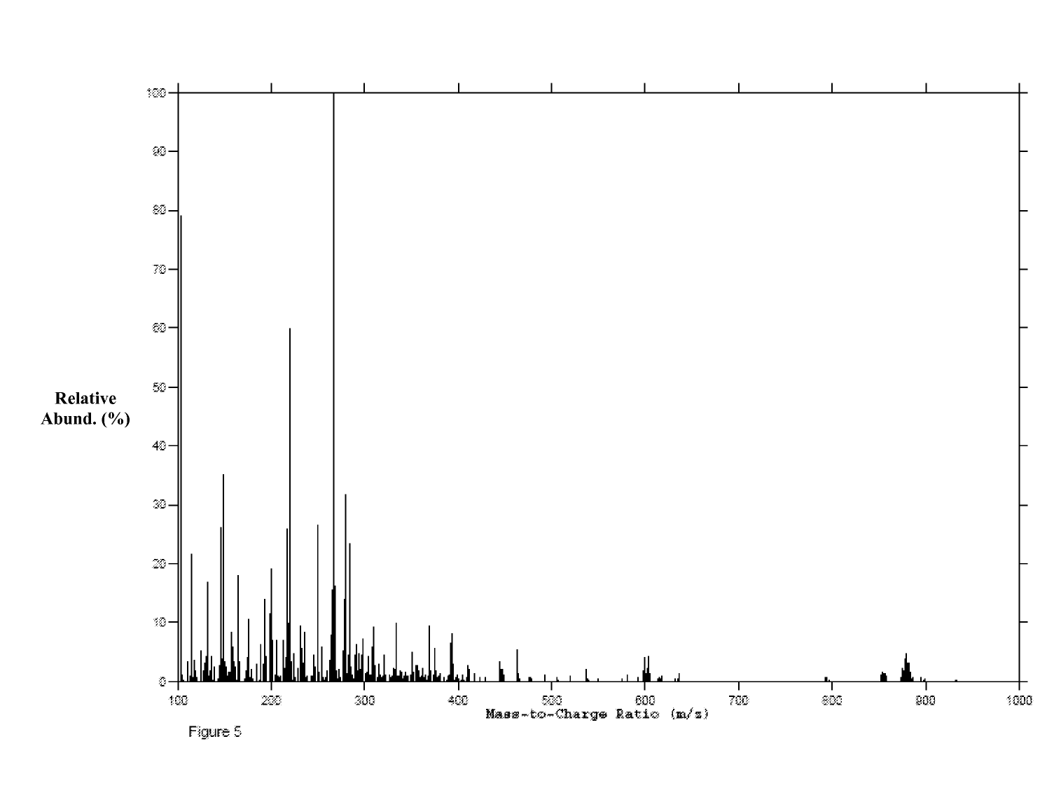

In some embodiments, the cranberry extract comprises a fraction comprising a Direct Analysis in Real Time (DART) mass spectrometry chromatogram of FIG. 5.

In some embodiments, any of the aforementioned extracts has an IC₅₀ value for C. albicans of less than 1000 μg/mL. In other embodiments, the IC₅₀ value for C. albicans is about 1 μg/mL to 500 μg/mL, 1 μg/mL to 100 μg/mL, or 1 μg/mL to 50 μg/mL.

In other embodiments, any of the aforementioned cranberry extracts has IC₅₀ value for E. coli of less than 500 μg/mL. In other embodiments, the IC₅₀ value for E. coli is about 0.05 to 100 μg/mL, or 0.05 to 50 μg/mL.

In some embodiments, the cranberry extract has an IC₅₀ value for S. aureus of less than 3000 μg/mL. In other embodiments, the IC₅₀ value for S. aureus is less than 2000 μg/mL, about 1 to 2000 μg/mL, 1 to 500 μg/m, 1 to 250 μg/mL, or 1 to 100 μg/mL. The S. aureus may or may not be a methicillin resistant S. aureus.

Another aspect of the invention relates to combined extracts of cranberry and cinnamon comprising an enriched amount of certain compounds having anti-infective activity, e.g., antibacterial and/or antifungal activity, e.g., activity against E. coli or S. aureus. In certain embodiments, the extract has been optimized for use for control of urinary tract infections. Certain embodiments of the extract are enriched in bioactive compounds that have been shown to inhibit E. coli and/or S. aureus adhesion and/or biofilm formation and its growth in vitro, representing two key anti-microbial properties that can control and mitigate urinary tract infections. In some embodiments, the present invention relates to a combined cranberry and cinnamon extract, comprising at least one compound selected from the group consisting of L-threonine, aminoevulinic acid, cinnamaldehyde, 4-hydroxybenzoic acid, athole/cuminaldehyde, chitosan, a-phenylindol, biotin, abscisic acid, vestitol, S-petasine, fraxin, and schisandrol B. In another embodiment, the combined extract comprises at least one of the aforementioned compounds in the following amounts: 0.001 to 5% by weight L-threonine, 0.01 to 5% by weight aminoevulinic acid, 0.5 to 10% cinnamaldehyde, 0.01 to 5% by weight 4-hydroxybenzoic acid, 0.01 to 5% by weight anethole/cuminaldehyde, 0.01 to 5% by weight chitosan, 0.05 to 10% by weight α-phenylindol, 0.01 to 5% by weight biotin, 0.05 to 10% by weight abscisic acid, 0.1 to 10% by weight vestitol, 0.01 to 5% S-petasine, 0.001 to 5% by weight fraxin, and 0.01 to 5% by weight schisandrol B. in other embodiments, the extract comprises at least one compound selected from 0.001 to 2% by weight L-threonine, 0.01 to 2% by weight aminoevulinic acid, 0.5 to 5% cinnamaldehyde, 0.01 to 2% by weight 4-hydroxybenzoic acid, 0.01 to 2% by weight anethole/cuminaldehyde, 0.01 to 2% by weight chitosan, 0.05 to 5% by weight α-phenylindol, 0.01 to 2% by weight biotin, 0.05 to 5% by weight abscisic acid, 0.1 to 5% by weight vestitol, 0.01 to 2% S-petasine, 0.001 to 2% by weight fraxin, and 0.01 to 2% by weight schisandrol B.

In some embodiments, the aforementioned extracts comprise at least one of the aforementioned compounds in the following amounts: 1 to 1000 μL-threonine, 5 to 1000 μg aminoevulinic acid, 500 to 5000 μg cinnamaldehyde, 10 to 1000 μg 4-hydroxybenzoic acid, 10 to 1000 μg anethole/cuminaldehyde, 10 to 1000 μg chitosan, 50 to 1500 μg a-phenylindol, 10 to 1500 μg biotin, 50 to 1500 μg abscisic acid, 50 to 2000 μg vestitol, 10 to 1500 μg S-petasine, 1 to 1000 μg fraxin, 10 to 1000 μg schisandrol B per 100 mg of extract.

In some embodiments, the aforementioned combined extract comprises aminoevulinic acid, L-threonine, cinnamaldehyde, 4-hydroxybenzoic acid, anethole/cuminaldehyde, chitosan, a-phenylindol, biotin, abscisic acid, vestitol, S-petasine, fraxin, and schisandrol B.

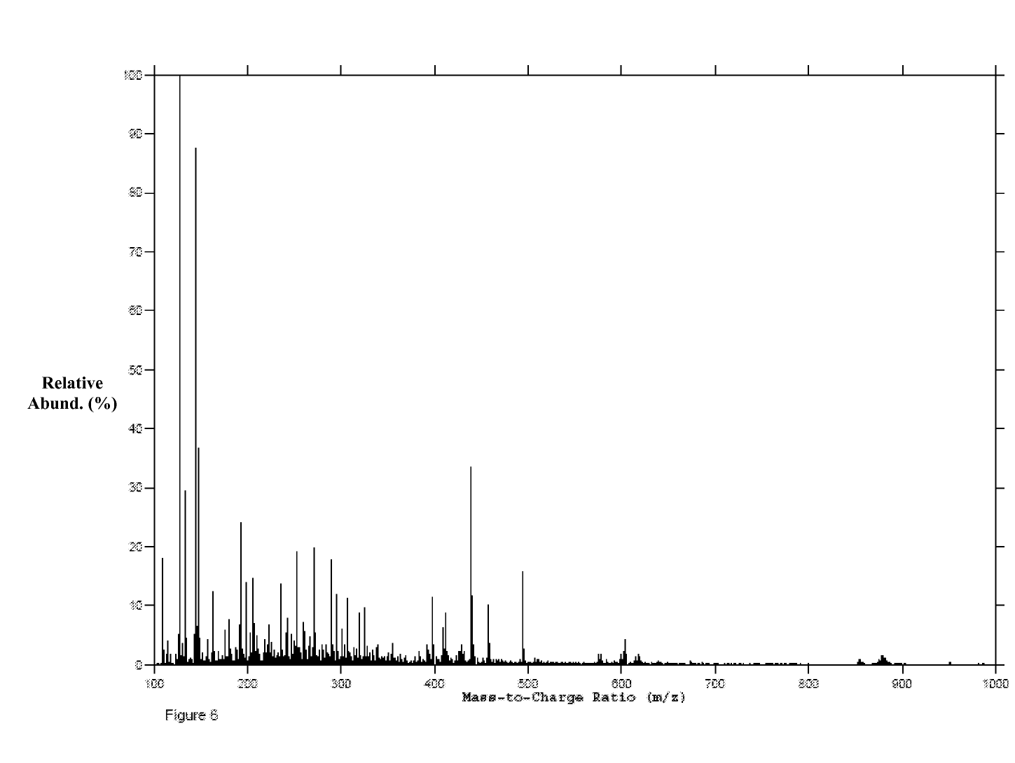

In some embodiments, the combined cranberry and cinnamon extract having a fraction comprising a Direct Analysis in Real Time (DART) mass spectrometry chromatogram of FIG. 6.

In some embodiments, any of the aforementioned extracts has an IC₅₀ value for C. albicans of less than 1000 μg/mL. In other embodiments, the IC₅₀ value for C. albicans is about 1 μg/mL to 500 μg/mL, 1 μg/mL to 100 μg/mL, or 1 μg/mL to 50 μg/mL.

In other embodiments, any of the aforementioned combined cranberry and cinnamon extracts has IC₅₀ value for E. coli of less than 500 μg/mL. In other embodiments, the IC₅₀ value for E. coli is about 0.05 to 100 μg/mL, or 0.05 to 50 μg/mL.

In some embodiments, the combined cranberry and cinnamon extract has an IC₅₀ value for S. aureus of less than 3000 μg/mL. In other embodiments, the IC₅₀ value for S. aureus is less than 2000 μg/mL, about 1 to 2000 μg/mL, 1 to 500 μg/mL, 1 to 250 μg/mL, or 1 to 100 μg/mL. The S. aureus may or may not be a methicillin resistant (MRSA) S. aureus.

In some embodiments, the cranberry extract is prepared by a process comprising:

a) providing a cranberry feedstock; and

b) extracting the cranberry feedstock with dimethylsulfoxide; and

c) isolating the extract.

In other embodiments, the process further comprises

d) providing a second cranberry feedstock

e) extracting the second feedstock with aqueous ethanol to form an aqueous ethanol extract;

f) separating the aqueous Ethanolic extract on a chromatography column with aqueous methanol;

g) collecting a 100% methanol fraction from the separation;

h) combining the methanol fraction of step g) with the extract of step c).

For example, the cranberry feedstock may be provided as sun-dried whole cranberry, which is then ground to powder with particle size at around 20-40 mesh. The resulting powder can be combined with DMSO and stirred, pulverized, or mashed in neat DMSO, followed by removal of the particulates to form the extract of step a) above. A second cranberry feedstock may be leached with aqueous ethanol, for example 40 to 99% ethanol, or 80% ethanol. The temperature of the leaching may be room temperature, or an elevated temperature, such as from about 25 to 60 degrees Celsius, or about 49 degrees Celsius. The resulting supernatant can be collected and isolated to provide the aqueous ethanol extract of step e). The extract can be loaded on to an adsorption column and separated using a methanol gradient. The aforementioned DMSO extract and Ethanolic extracts can be combined to provide a final extract composition.

The present invention also relates to methods of treating or preventing an infection, comprising administering to a subject in need thereof a therapeutically effective amount of any of the aforementioned cranberry or combined cranberry and cinnamon extracts. In some embodiments, the infection is a bacterial infection or a fungal infection. For example, the infection may be selected from the group consisting of C. albicans, E. coli, or S. aureus. In some embodiments, the infection is a yeast infection, while in other embodiments, the infection is a Staph infection or a methicillin resistant (MRSA) S. aureus infection. In other embodiments, the infection is a urinary tract infection.

Pharmaceutical Compositions

In some aspects of the invention, pharmaceutical formulations comprising any of the aforementioned cranberry extracts and at least one pharmaceutically acceptable carrier are provided. In other aspects, the pharmaceutical composition comprises any of the aforementioned cranberry extracts, any of the aforementioned cinnamon extracts, and pharmaceutically acceptable carrier.

Compositions of the disclosure comprise extracts of cranberry and optionally cinnamon in forms such as a paste, powder, oils, liquids, suspensions, solutions, ointments, or other forms, comprising, one or more fractions or sub-fractions to be used as dietary supplements, nutraceuticals, or such other preparations that may be used to prevent or treat various human ailments. The extracts can be processed to produce such consumable items, for example, by mixing them into a food product, in a capsule or tablet, or providing the paste itself for use as a dietary supplement, with sweeteners or flavors added as appropriate. Accordingly, such preparations may include, but are not limited to, cranberry extract preparations for oral delivery in the form of tablets, capsules, lozenges, liquids, emulsions, dry flowable powders and rapid dissolve tablet. The cranberry extracts may advantageously be formulated into a suppository or lozenge for vaginal administration. Based on the anti-fungal activities described herein, patients would be expected to benefit from daily dosages in the range of from about 50 mgs to about 1000 mg. For example, a lozenge comprising about 50, 55, 60, 65, 70, 75, 80, 85, 90, 95, 100, 105, 110, 115, 120, 125, 130, 135, 140, 145, 150, 160, 170, 180, 190, 200, 210, 220, 230, 240, or 250 mg of the extract can be administered once or twice a day to a subject as a prophylactic. Alternatively, in response to a severe allergic reaction, two lozenges may be needed every 4 to 6 hours.

In one embodiment, a dry extracted cranberry composition is mixed with a suitable solvent, such as but not limited to water or ethyl alcohol, along with a suitable food-grade material using a high shear mixer and then spray air-dried using conventional techniques to produce a powder having grains of cranberry extract particles combined with a food-grade carrier.

In a particular example, cranberry extract composition is mixed with about twice its weight of a food-grade carrier such as maltodextrin having a particle size of between 100 to about 150 micrometers and an ethyl alcohol solvent using a high shear mixer. Inert carriers, such as silica, preferably having an average particle size on the order of about 1 to about 50 micrometers, can be added to improve the flow of the final powder that is formed. Preferably, such additions are up to 2% by weight of the mixture. The amount of ethyl alcohol used is preferably the minimum needed to form a solution with a viscosity appropriate for spray air-drying. Typical amounts are in the range of between about 5 to about 10 liters per kilogram of extracted material. The solution of extract, maltodextrin and ethyl alcohol is spray air-dried to generate a powder with an average particle size comparable to that of the starting carrier material.

In another embodiment, an extract and food-grade carrier, such as magnesium carbonate, a whey protein, or maltodextrin are dry mixed, followed by mixing in a high shear mixer containing a suitable solvent, such as water or ethyl alcohol. The mixture is then dried via freeze drying or refractive window drying. In a particular example, extract material is combined with food grade material about one and one-half times by weight of the extract, such as magnesium carbonate having an average particle size of about 20 to 200 micrometers. Inert carriers such as silica having a particle size of about 1 to about 50 micrometers can be added, preferably in an amount up to 2% by weight of the mixture, to improve the flow of the mixture. The magnesium carbonate and silica are then dry mixed in a high speed mixer, similar to a food processor-type of mixer, operating at 100's of rpm. The extract is then heated until it flows like dense oil. Preferably, it is heated to about 50° C. The heated extract is then added to the magnesium carbonate and silica powder mixture that is being mixed in the high shear mixer. The mixing is continued preferably until the particle sizes are in the range of between about 250 micrometers to about 1 millimeter. Between about 2 to about 10 liters of cold water (preferably at about 4° C.) per kilogram of extract is introduced into a high shear mixer. The mixture of extract, magnesium carbonate, and silica is introduced slowly or incrementally into the high shear mixer while mixing. An emulsifying agent such as carboxymethylcellulose or lecithin can also be added to the mixture if needed. Sweetening agents such as Sucralose or Acesulfame K up to about 5% by weight can also be added at this stage if desired. Alternatively, extract of Stevia rebaudiana, a very sweet-tasting dietary supplement, can be added instead of or in conjunction with a specific sweetening agent (for simplicity, Stevia will be referred to herein as a sweetening agent). After mixing is completed, the mixture is dried using freeze-drying or refractive window drying. The resulting dry flowable powder of extract, magnesium carbonate, silica and optional emulsifying agent and optional sweetener has an average particle size comparable to that of the starting carrier and a predetermined extract.

According to another embodiment, an extract is combined with approximately an equal weight of food-grade carrier such as whey protein, preferably having a particle size of between about 200 to about 1000 micrometers. Inert carriers, such as silica, having a particle size of between about 1 to about 50 micrometers, or carboxymethylcellulose having a particle size of between about 10 to about 100 micrometers can be added to improve the flow of the mixture. Preferably, an inert carrier addition is no more than about 2% by weight of the mixture. The whey protein and inert ingredient are then dry mixed in a food processor-type of mixer that operates over 100 rpm. The extract can be heated until it flows like dense oil (preferably heated to about 50° C.). The heated extract is then added incrementally to the whey protein and inert carrier that is being mixed in the food processor-type mixer. The mixing of the extract and the whey protein and inert carrier is continued until the particle sizes are in the range of about 250 micrometers to about 1 millimeter. Next, 2 to 10 liters of cold water (preferably at about 4° C.) per kilogram of the paste mixture is introduced in a high shear mixer. The mixture of extract, whey protein, and inert carrier is introduced incrementally into the cold water containing high shear mixer while mixing. Sweetening agents or other taste additives of up to about 5% by weight can be added at this stage if desired. After mixing is completed, the mixture is dried using freeze drying or refractive window drying. The resulting dry flowable powder of extract, whey protein, inert carrier and optional sweetener has a particle size of about 150 to about 700 micrometers and a unique predetermined extract.

In the embodiments where the extract is to be included into an oral fast dissolve tablet as described in U.S. Pat. No. 5,298,261, the unique extract can be used “neat,” that is, without any additional components which are added later in the tablet forming process as described in the patent cited. This method obviates the necessity to take the extract to a dry flowable powder that is then used to make the tablet.

Once a dry extract powder is obtained, such as by the methods discussed herein, it can be distributed for use, e.g., as a dietary supplement or for other uses. In a particular embodiment, the novel extract powder is mixed with other ingredients to form a tableting composition of powder that can be formed into tablets. The tableting powder is first wet with a solvent comprising alcohol, alcohol and water, or other suitable solvents in an amount sufficient to form a thick doughy consistency. Suitable alcohols include, but not limited to, ethyl alcohol, isopropyl alcohol, denatured ethyl alcohol containing isopropyl alcohol, acetone, and denatured ethyl alcohol containing acetone. The resulting paste is then pressed into a tablet mold. An automated tablet molding system, such as described in U.S. Pat. No. 5,407,339, can be used. The tablets can then be removed from the mold and dried, preferably by air-drying for at least several hours at a temperature high enough to drive off the solvent used to wet the tableting powder mixture, typically between about 70° to about 85° C. The dried tablet can then be packaged for distribution

Compositions can be in the form of a paste, resin, oil, powder or liquid. Liquid preparations for oral administration may take the form of, for example, solutions, syrups or suspensions, or they may be presented as a dry product for reconstitution with water or other suitable vehicle prior to administration. Such liquid preparations may be prepared by conventional means with pharmaceutically acceptable additives such as suspending agents (e.g., sorbitol syrup, methyl cellulose, or hydrogenated edible fats); emulsifying agents (e.g., lecithin or acacia); non-aqueous vehicles (e.g., almond oil, oily esters or ethyl alcohol); preservatives (e.g., methyl or propyl p-hyroxybenzoates or sorbic acid); and artificial or natural colors and/or sweeteners. Compositions of the liquid preparations can be administered to humans or animals in pharmaceutical carriers known to those skilled in the art. Such pharmaceutical carriers include, but are not limited to, capsules, lozenges, syrups, sprays, rinses, and mouthwash.

Dry powder compositions may be prepared according to methods disclosed herein and by other methods known to those skilled in the art such as, but not limited to, spray air drying, freeze drying, vacuum drying, and refractive window drying. The combined dry powder compositions can be incorporated into a pharmaceutical carrier such, but not limited to, tablets or capsules, or reconstituted in a beverage such as a tea.

The described extracts may be combined with extracts from other plants such as, but not limited to, varieties of Gymnema, turmeric, Boswellia, guarana, cherry, lettuce, Echinacea, piper betel leaf, Areca catechu, Muira puama, ginger, willow, suma, kava, horny goat weed, Ginkgo biloba, mate, garlic, puncture vine, arctic root, astragalus, Eucommia, Cinnamomum, Cassia, and Uncaria, or pharmaceutical or nutraceutical agents.

A tableting powder can be formed by adding about 1 to 40% by weight of the powdered extract, with between 30 to about 80% by weight of a dry water-dispersible absorbent such as, but not limited to, lactose. Other dry additives such as, but not limited to, one or more sweetener, flavoring and/or coloring agents, a binder such as acacia or gum arabic, a lubricant, a disintegrant, and a buffer can also be added to the tableting powder. The dry ingredients are screened to a particle size of between about 50 to about 150 mesh. Preferably, the dry ingredients are screened to a particle size of between about 80 to about 100 mesh.

Preferably, the tablet exhibits rapid dissolution or disintegration in the oral cavity. The tablet is preferably a homogeneous composition that dissolves or disintegrates rapidly in the oral cavity to release the extract content over a period of about 2 seconds or less than 60 seconds or more, preferably about 3 to about 45 seconds, and most preferably between about 5 to about 15 seconds.

Various rapid-dissolve tablet formulations known in the art can be used. Representative formulations are disclosed, for example, in U.S. Pat. Nos. 5,464,632; 6,106,861; 6,221,392; 5,298,261; and 6,200,604; the entire contents of each are expressly incorporated by reference herein. For example, U.S. Pat. No. 5,298,261 teaches a freeze-drying process. This process involves the use of freezing and then drying under a vacuum to remove water by sublimation. Preferred ingredients include hydroxyethylcellulose, such as Natrosol from Hercules Chemical Company, added to between 0.1 and 1.5%. Additional components include maltodextrin (Maltrin, M-500) at between 1 and 5%. These amounts are solubilized in water and used as a starting mixture to which is added the cranberry extraction composition, along with flavors, sweeteners such as Sucralose or Acesulfame K, and emulsifiers such as BeFlora and BeFloraPlus which are extracts of mung bean. A particularly preferred tableting composition or powder contains about 10 to 60% by of the extract powder and about 30% to about 60% of a water-soluble diluent.

In a preferred implementation, the tableting powder is made by mixing in a dry powdered form the various components as described above, e.g., active ingredient (extract), diluent, sweetening additive, and flavoring, etc. An overage in the range of about 10% to about 15% of the active extract can be added to compensate for losses during subsequent tablet processing. The mixture is then sifted through a sieve with a mesh size preferably in the range of about 80 mesh to about 100 mesh to ensure a generally uniform composition of particles.

The tablet can be of any desired size, shape, weight, or consistency. The total weight of the extract in the form of a dry flowable powder in a single oral dosage is typically in the range of about 40 mg to about 1000 mg. In a preferred form, the tablet is a disk or wafer of about 0.15 inch to about 0.5 inch in diameter and about 0.08 inch to about 0.2 inch in thickness, and has a weight of between about 160 mg to about 1,500 mg. In addition to disk, wafer or coin shapes, the tablet can be in the form of a cylinder, sphere, cube, or other shapes.

Compositions of unique extract compositions may also comprise extract compositions in an amount between about 10 mg and about 2000 mg per dose.

### Exemplification

The disclosure now being generally described, it will be more readily understood by reference to the following examples, which are included merely for purposes of illustration of certain aspects and embodiments of the disclosure, and are not intended to limit the disclosure.

Cranberry Extracts, Cinnamon and Cranberry/Cinnamon blends

A. Extraction

Sun-dried whole cranberry, purchased from Cranberry Hill Farm (USA), were ground to powder with particle size at around 20-40 mesh. The resulting powder was mashed in neat DMSO and the particulates were precipitated by centrifugation at 1500×g for 10 minutes (Extract 1). Approximately 15 g of ground cranberry were extracted by leaching with aqueous 80% ethanol at 40° C. The leaching experiment was performed using 2 stages at solvent/feed ratio of 15 and 10 respectively and 2 hours in each stage. After extraction, the extracted slurry was filtered off by Fisher brand P4 filter paper with port size of 4-8 μm and centrifuged at 537×g for 20 minutes. The supernatant was collected and dried to a powder to be loaded on an adsorption column. The polymer adsorbent processing was carried out at room temperature. Firstly, 320 g of XAD 7HP was washed with ethanol to remove monomer and impurity and then soaked in distilled water overnight before packing. Following the column packing, 800 mg of the dried aqueous ethanol extract were resuspended in a water solution at a concentration of 5% (w/v) and loaded onto the XAD 7HP column with a flow rate of 1.7 BV/h. After loading, 1000 mL of water were used to wash the column at the flow rate of 2.0 BV/h. The desorption was performed with 1000 mL of 80% ethanol. The obtained whole fraction (Extract 2) was dried in preparation for the separation on a Sephadex LH-20 column with an internal diameter of 5 cm and height of 17 cm with a bed volume of 340 mL. Dried Extract 2 was dissolved in 40% aqueous methanol. The solution was filtered by 0.22 μm to remove small particulates to obtain the loading solution at concentration of 5% (w/v). The solution loaded on the column was eluted by using mobile phase of (A) water and (B) methanol. The fractions corresponding to 60% methanol (Extract 3) and 100% methanol (Extract 4) were collected and dried. Extract 1 and Extract 4 were resuspended in neat DMSO and blended in a 200:1 ratio (Extract 5). Cinnamon bark was extracted with 80% (v/v) ethanol at 40° C. and the resulting extract was blended in a 10:1 ratio of Extract 5 to cinnamon (Extract 6). All extracts were lyophilized and were utilized as dried powders for DART TOF-MS analyses as well as for all in vitro bioassay evaluations.

B. DART TOF-MS Characterization of Extracts

A Jeol DART AccuTOF-MS (Model JMS-T100LC; Jeol USA, Peabody, Mass.) was used for chemical characterization of cranberry, cinnamon and combination extracts. The DART settings were loaded as follows: DART Needle voltage=3000V; Electrode 1 voltage=150V; Electrode 2 voltage=250V; Temperature=250° C.; He Flow Rate=3.12 LPM. The following AccuTOF mass spectrometer settings were loaded: Ring Lens voltage=5 V; Orifice 1 voltage=10 V; Orifice 2 voltage=5 V; Peaks voltage=1000 V (for resolution between 100-1000 amu); Orifice 1 temperature was turned off. The samples were introduced by placing the closed end of a borosilicate glass capillary tube into the extracts, and the coated capillary tube was placed into the DipIT® sample holder providing a uniform and constant surface exposure for ionization in the He plasma. The extracts were allowed to remain in the He plasma stream until signal was observed in the total-ion-chromatogram (TIC). The sample was removed and the TIC was brought down to baseline levels before the next sample was introduced. A polyethylene glycol 600 (Ultra Chemicals, Kingston R.I.) was used as an internal calibration standard giving mass peaks throughout the desired range of 100-1000 amu.

C. Microbial Strains

All the assays were performed using a vaginal isolate of Candida albicans (ATCC 96133), a methicillin resistant strain (MRSA) of Staphylococcus aureus (ATCC 700787), and a urinary tract isolate strain of Escherichia coli (ATCC 53499). All microbial strains were obtained from the American Type Culture Collection (ATCC; Manassas, Va.). Media used for the growth of the bacterial and fungal cell lines were Trypticase Soy Broth (TSB) and Trypticase Soy Broth with 0.6% Yeast Extract (TSB-YE), respectively (Difco, Md.).

D. Microbial Growth Inhibition

For E. coli and S. aureus cultures, a 5× and 1× solution of TSB was prepared. For Candida, filter sterilized solutions of 1× and 5× TSB-YE for C. albicans were prepared. Overnight cultures of E. coli and S. aureus were grown at 32° C. in 1×TSB. Overnight cultures of C. albicans were also grown overnight at 32° C. in 1×TSB-YE. Multiple dilutions of the chemistries were prepared in a 1% (v/v) DMSO Tris-Buffered Saline solution (TBS; pH 7.4). Aliquots (60 μL) of the extract solutions, 20 μL of E. coli, S. aureus or C. albicans and 20 μL of 5× media added to each well of a Nunc polystyrene 384 well plate (Nunc, N.Y.). Cells were grown in the wells overnight at 32° C. while absorbance at 600 nm (a measure of growth) was monitored every 20 minutes in a BioTek Synergy 4 microplate reader (BioTek, Winooski, Vt.).

E. Adhesion/Biofilm Formation Assays

The adhesion assay was conducted as described previously (R. S. Alberte and R. D. Smith, 2006. Generation of combinatorial synthetic libraries and screening for novel proadhesins and antiadhesins, U.S. Pat. No. 7,132,567). Cell suspensions were prepared by spinning down (centrifugation at 500×g for 5 minutes) overnight cultures of S. aureus, E. coli and C. albicans as described above. To yield an OD₆₀₀ reading of 0.2-0.25, cells were resuspended in Tris Buffered Saline (TBS, pH 7.4). Dilutions of extracts were also established in 1% (v/v) DMSO-TBS. Aliquots (200 μL) of extract solutions were added to micro titer plate wells. Aliquots (50 μL) of microbial suspensions were added to each well and plates were incubated at room temperature for one hour for E. coli and S. aureus, and 2 hours for C. albicans to allow the cells to adhere to the well bottoms. After incubation, plates were washed with PBS three times to remove non-adherent and loosely adherent cells. Cells were fixed for staining with 70% (v/v) ethanol (USP) for 1 minute. Each well was covered with 100 μL of the fluorescent nucleic acid staining dye Syto 13 (Invitrogen, Carlsbad, Calif.), and incubated for 15 minutes. The plates were read in either a Tecan M200 microplate reader (Tecan Inc., Research Triangle Park, N.C.) or a Synergy 4 plate reader (Biotek, Winooski, Vt.), with excitation and emission wavelengths of 485 and 535 nm, respectively, to quantify adhered cells in each well relative to control wells.

F. Direct Binding Assay

A Direct Binding Assay (B. Roschek Jr., R. C. Fink, M. D. McMichael, D. Li and R. S. Alberte, 2009. Elderberry flavonoids bind to and prevent H1N1 Infection in vitro. Phytochemistry. In Press). was used to determine which bioactive chemicals from the cranberry extracts bind to the microbes blocking adhesion. The assay involved the incubation of the microorganisms with the cranberry extracts as described above. The microbial cells were centrifuged and the supernatant containing unbound chemicals was removed. The cells were re-suspended in PBS (pH 7.4) and centrifuged, and the supernatant containing excess unbound chemicals was removed. This process was repeated 4 times to remove unbound chemistries. The cells were collected, fixed in 100% (USP) ethanol to kill the pathogens, and analyzed by DART TOF-MS using the same settings as for the chemical characterization of the extracts.

G. Post-binding Assay

Extracts and cultures of C. albicans, E. coli, and S. aureus were prepared as previously described for the adhesion assay in buffer. Serial dilutions of the extracts were prepared to generate final concentrations of 1000, 100, and 0 μg mL⁻¹. The initial solutions comprised of cells with or without extracts were prepared in deep well plates (2 mL per well), with the 0 μg mL⁻¹ wells as positive controls. The experiment was performed in quadruplicates for each organism.

The deep well plates were incubated for 1 hour at room temperature. After the incubation, 200 μL of each of the deep well plates were added to corresponding high binding plates. These new plates were incubated at room temperature for one hour to allow for the adhesion of the cells. The plates were then washed following procedures described in the adhesion assay. The plates subjected to the direct binding assay were centrifuged at 500×g for 10 minutes and washed with PBS. After these were incubated for one hour at room temperature they were also washed following procedures described in the adhesion assay. The plates (experimental and control wells) were stained with Syto 13 dye and the adhesion of the cells was quantified measuring the fluorescence emission at 530 nm with 485 nm excitation in a microplate reader (BioTek, Winooski, Vt.).

H. Identification and Characterization of Known Bioactive Chemistries

The DART-MS spectrum of each extract was analyzed for the [M+H]⁺ ions were held to within 10 mmu of the calculated masses. The identified compounds are reported with greater than 90% confidence. Chemical structures were confirmed by elemental composition and isotope matching programs in the Jeol MassCenterMain Suite software. In addition, molecular identification were searched and verified against the NIST/NIH/EPA Mass Spec Database when needed.

I. Human Pharmacokinetic Studies

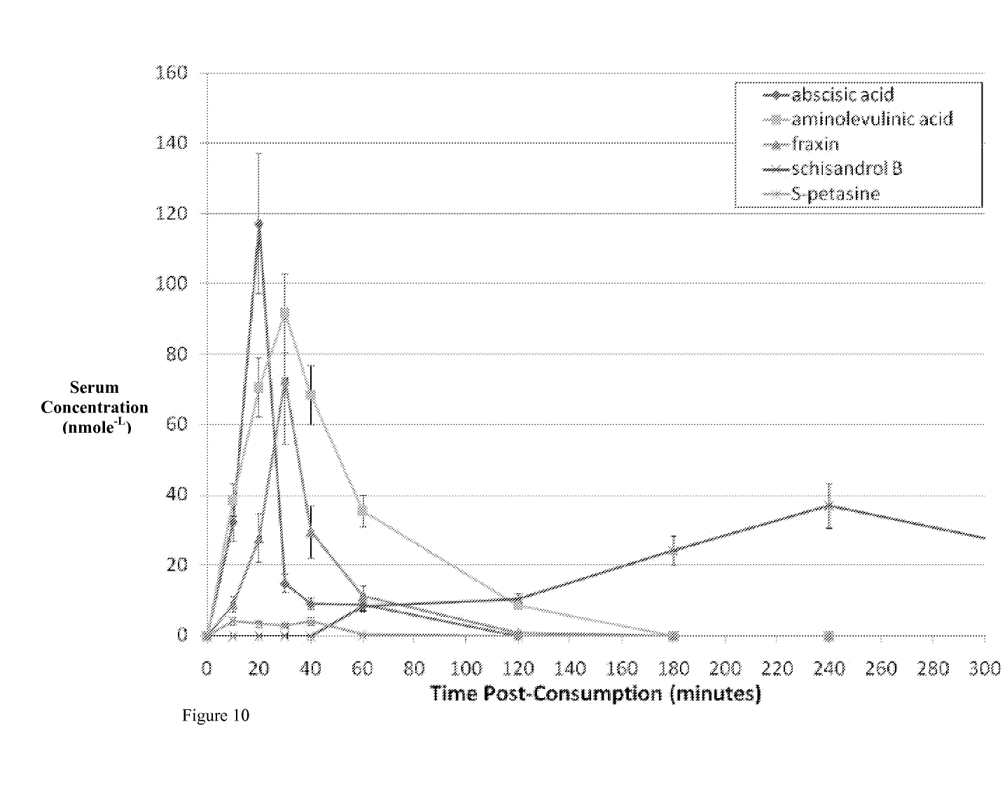

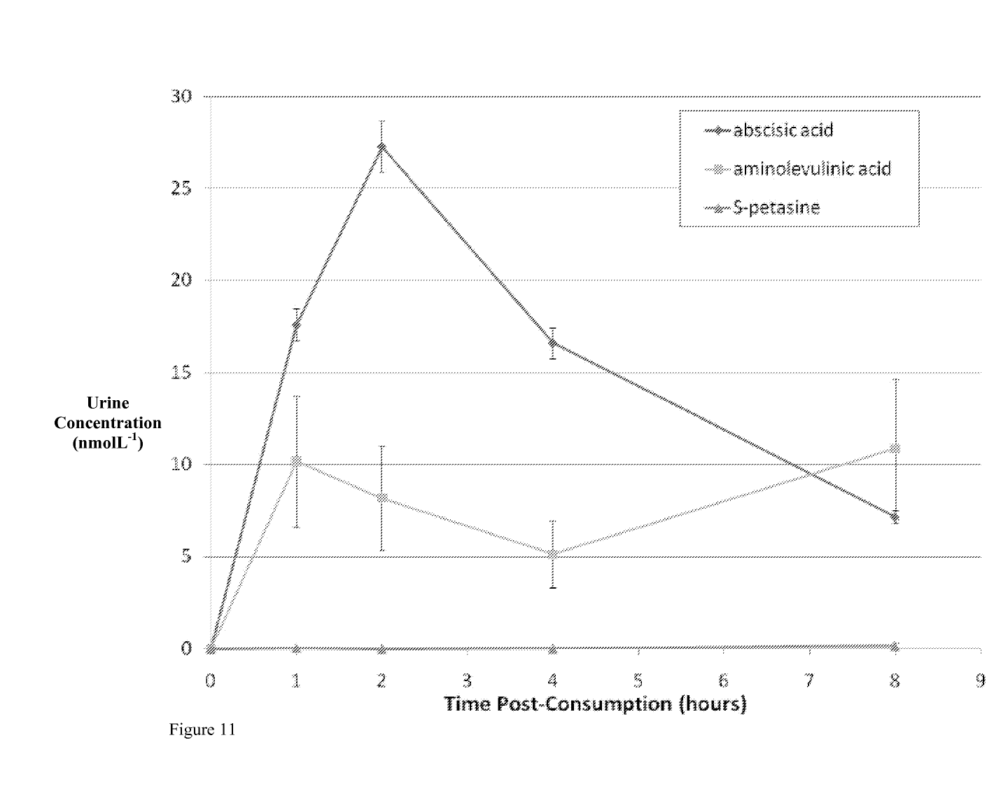

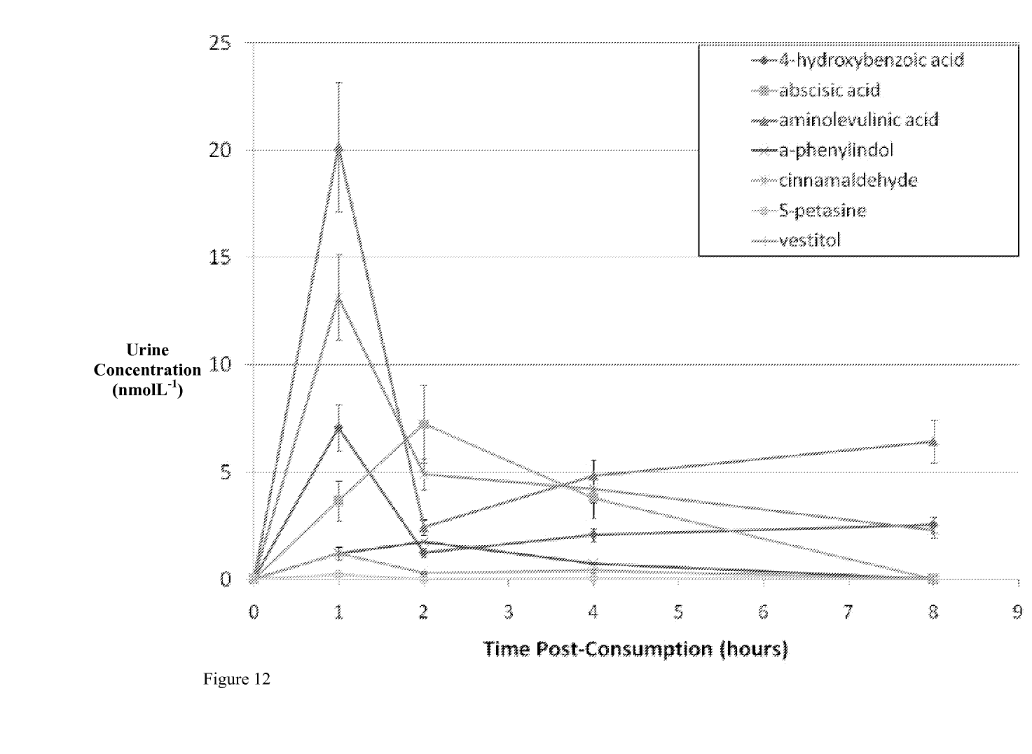

Cranberry extracts 5 and 6 were prepared by HerbalScience Singapore Pte. Ltd. and prepared as 150-mg and 140-mg capsules, respectively. Each pharmacokinetic study (1 per extract) consisted of five healthy consenting adults ranging in age from 25 to 50 were instructed not to consume foods rich in phenolics 24 hours prior to the initiation of the study. A certified individual collected urine samples at several time intervals between 0 and 480 minutes after two capsules of a cranberry extract were ingested immediately after the time zero time point. Blood samples were handled with approved protocols and precautions, centrifuged to remove cells and the serum fraction was collected and frozen. Blood was not treated with heparin to avoid any analytical interference. Serum samples were stored frozen at −20° C. until analysis. The serum was extracted with an equal volume of neat ethanol (USP) to minimize background of proteins, peptides, and polysaccharides present in serum. The ethanol extract was centrifuged at 9300×g for 10 minutes at 4° C., the supernatant was removed, concentrated to 200 μL volume which was then used for DART TOF-MS analyses (FIG. 10). Urine samples were handled with approved protocols and precautions and frozen. Serum samples were stored frozen until analysis. The urine samples were analyzed neat by DART TOF-MS (FIGS. 11 and 12).

Results

A. DART TOF-MS and Chemical Characterization of Extracts

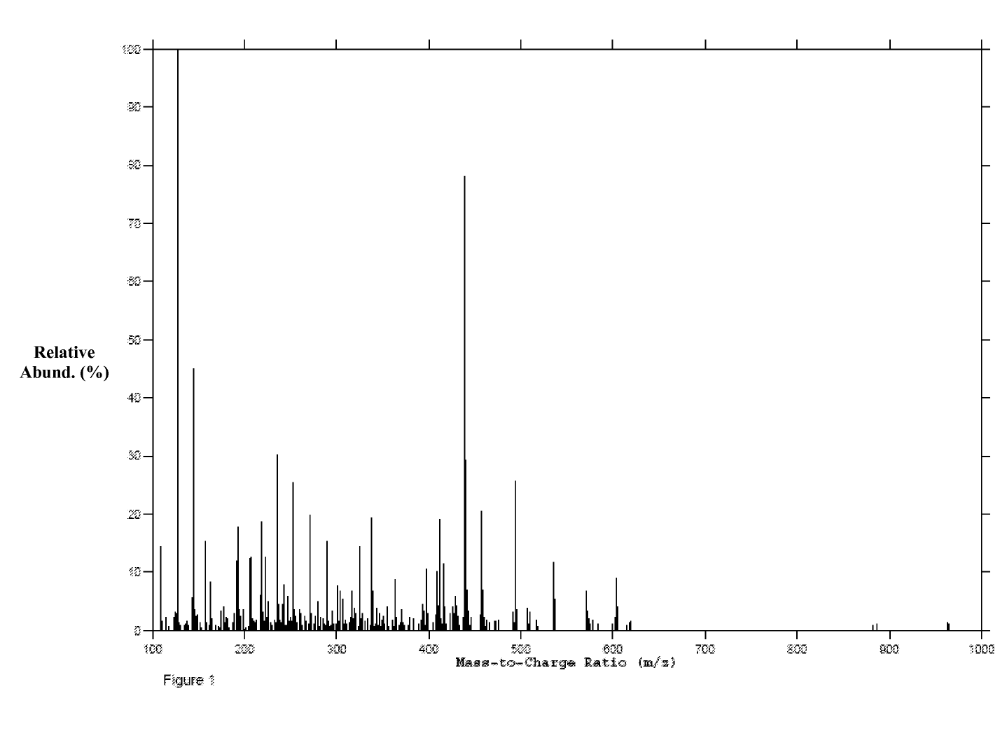

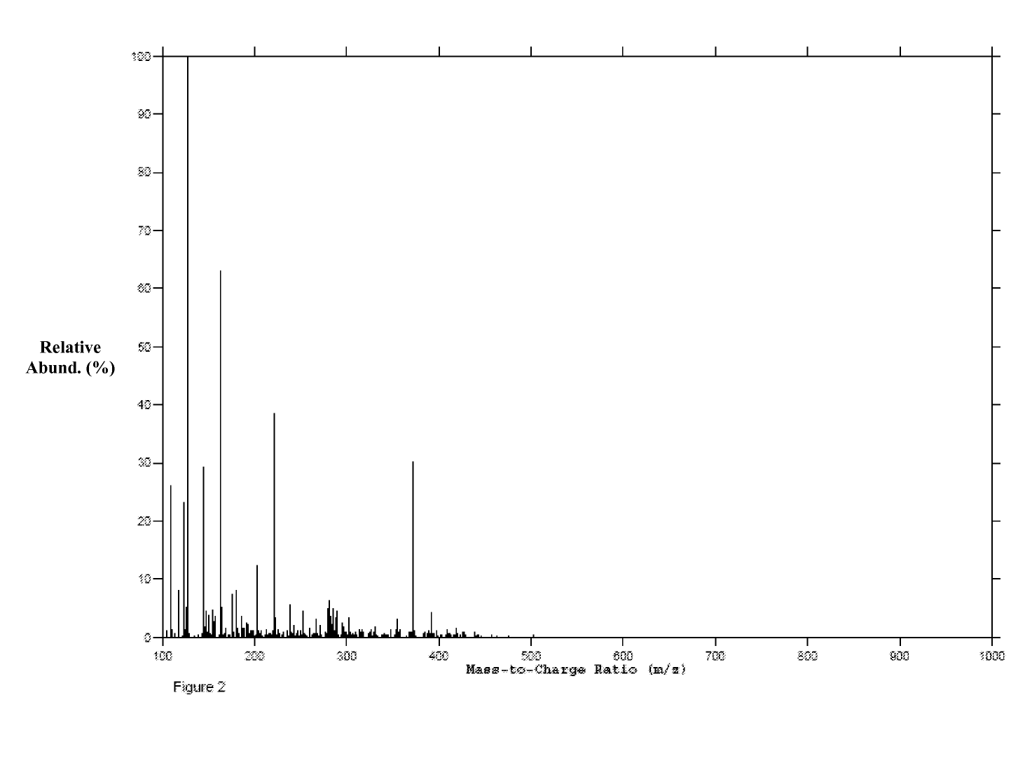

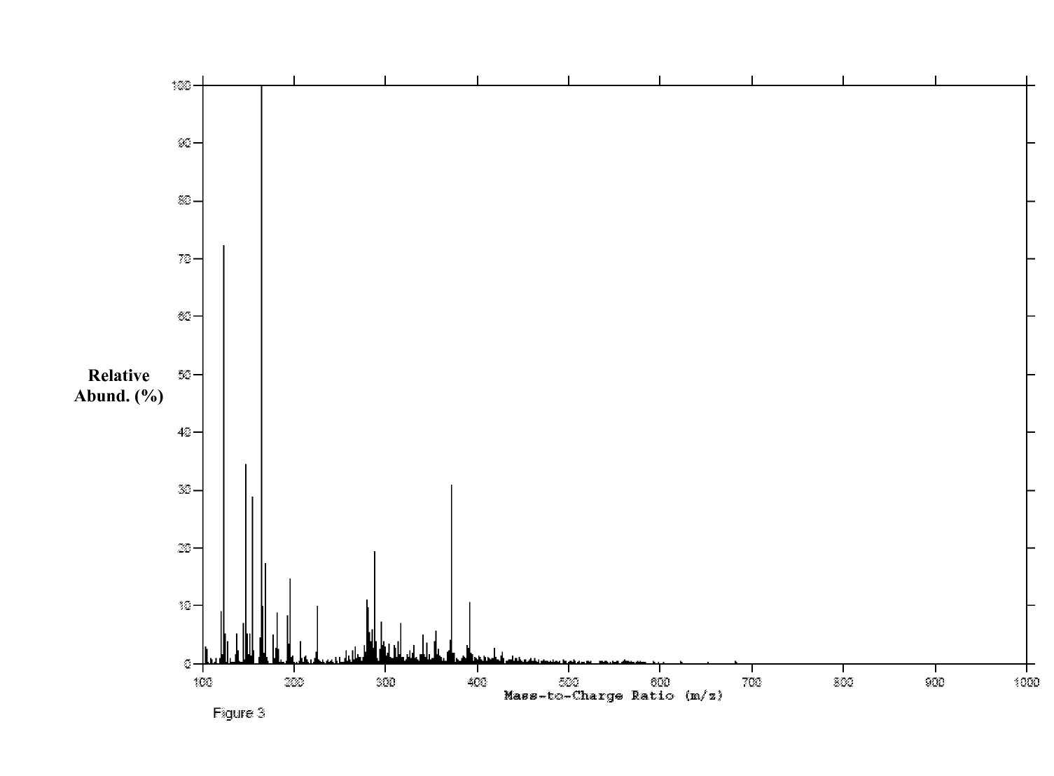

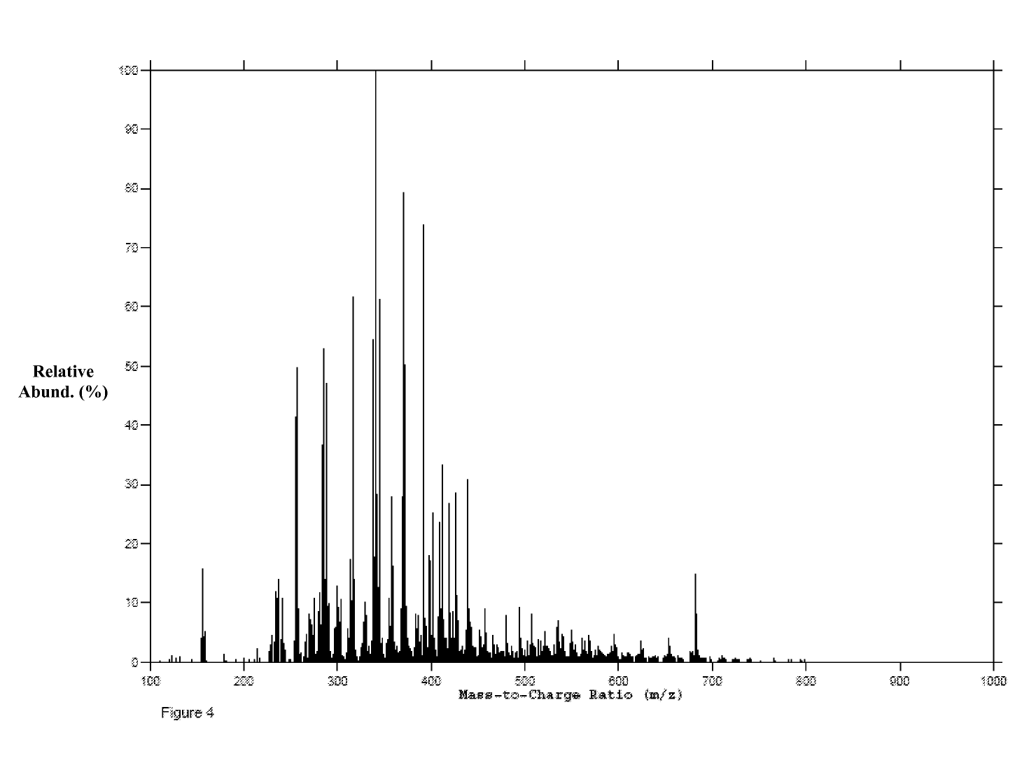

In FIG. 1, the DART TOF-MS of Extract 1 is shown with the mass distribution (amu; X-axis) and the relative abundances (%; Y-axis) of each compound detected. FIGS. 2 through 6 show the DART TOF mass spectra of cranberry Extract 2 through 6, respectively. Some of the more abundant identified compounds (>15% relative abundance) in the cranberry extract included adenine, pyrogallol, glutaric acid, nornicotine, levoglucosan, synephrine, aminobutyric acid, and 4-methyl-7-ethoxycoumarin. Vitamin B₅, pantothenic acid, was unusually abundant (60% relative abundance), and it is well known for its critical role critical in the metabolism and synthesis of carbohydrates, proteins, and fats. Magnolol, also very abundant in the extract, is known to function as anti-inflammatory (NF-kB) and as an inhibitor of angiogenesis of cancerous tumors. Tables 1 through 6 below summarize the compounds identified in each of the 6 cranberry extracts disclosed here.

TABLE 1
Summary of the identified compounds in Extract 1 as determined
by DART TOF-MS.

| | | Relative |
|---|---|---|
| | Measured | Abundance |
| Compound Name | Mass | (%) |
| 3-Methyl-2-butenoic acid | 101.0548 | 0.2527 |
| 3-Pyridinecarboxylic acid: Nitrile | 105.0355 | 0.5425 |
| Diethanolamine | 106.0866 | 0.5470 |
| 1,2-Dimethylbenzene | 107.0822 | 0.7117 |
| 1,4-Benzoquinone | 109.0281 | 27.9197 |
| 2-Aminoethanesulfinic acid | 110.0372 | 2.7247 |
| 1,4-Benzenediol | 111.0443 | 8.0273 |
| Cytosine; OH-form | 112.0558 | 0.6115 |
| Uracil | 113.0346 | 2.7791 |
| 2-Propenoic acid: Isopropylamide | 114.0906 | 4.4558 |
| 3,4-Dihydroxy-2-methylenebutanoicacid | 115.0390 | 4.9286 |
| (S)-form: Lactone | | |
| 2-(Aminomethyl)-2-propenoic acid: Me | 116.0752 | 1.0362 |
| ester | | |
| 4,5-Dihydro-2-methylthiazole: N-Me | 117.0548 | 2.1832 |
| 2-(Dimethylamino)ethanol: Et ether | 118.1232 | 2.0708 |
| 2-Methylaminoacetic acid: N-Nitroso | 119.0539 | 0.3515 |
| 3-Methylbutanoic acid: Chloride | 121.0374 | 1.4382 |
| Benzoic acid | 123.0530 | 2.6972 |
| (2- | 124.0525 | 1.0411 |
| Hydroxyethyl)dimethylsulfoxonium(1+) | | |
| Hydroxy-1,4-benzoquinone | 125.0305 | 1.3153 |
| 3,4-Dihydroxy-2-methylpyridine; Di-OH- | 126.0501 | 3.0023 |
| form | | |
| 5-Hydroxy-3-vinyl-2(5H)-furanone | 127.0387 | 100.0000 |
| 2-Ethyl-4-methylthiazole | 128.0444 | 4.9260 |
| 5,6-Dihydro-5-hydroxy-6-methyl-2H- | 129.0526 | 6.0205 |
| pyran-2-one; (5R,6S)-form | | |
| 3,4-Dihydro-4-hydroxy-2H-pyrrole-2- | 130.0588 | 1.0923 |
| carboxylic acid | | |
| 2-Nonene-4,6,8-triyn-1-ol | 131.0554 | 1.0528 |
| 2-Methyl-3,4-piperidinediol | 132.0993 | 0.4603 |
| Thiourea: N,N-Di-Et | 133.0759 | 1.4400 |
| 4-Mercapto-2-butanone S-Me, S-oxide | 135.0398 | 4.6456 |
| 2-Amino-3,4-dihydroxybutanoic acid; | 136.0662 | 1.4618 |
| (2R,3S)-form | | |
| 4-Methylbenzoic acid | 137.0661 | 1.9741 |
| 3-(Methylthio)propylamine S,S-Dioxide | 138.0660 | 0.4639 |
| 2-Hydroxybenzoic acid | 139.0428 | 6.5696 |
| 3,4-Dihydroxybenzylamine | 140.0708 | 1.6346 |
| Norzooanemonin | 141.0694 | 1.1926 |
| 4-Amino-2-hydoxy-5- | 142.0684 | 0.6289 |
| (hydroxymethyl)pyrimidine | | |
| 2-Hydroxy-2-hydroxymethyl-4- | 143.0369 | 2.3310 |
| cyclopentene-1,3-dione | | |
| 2-Hydroxymethylclavam | 144.0597 | 2.8404 |
| 2,3-Dihydro-3,6-dihydroxy-2-methyl-4H- | 145.0494 | 98.4252 |
| pyran-4-one | | |
| 1H-Indole-3-carboxaldehyde | 146.0554 | 7.6606 |
| 2,5-Furandiacetic acid: Dinitrile | 147.0619 | 15.4765 |
| 2-Hydroxybenzoic acid: Et ether, nitrile | 148.0711 | 1.5117 |
| 4,6,8-Nonatriyne-1,2-diol | 149.0688 | 2.2265 |
| 4-Methylbenzoic acid: Methylamide | 150.0851 | 0.5132 |
| 4-Methylbenzoic acid: Me ester | 151.0766 | 3.6184 |
| Ethyl-1,4-benzoquinone: 4-Oxime | 152.0732 | 1.0190 |
| 2-Vinyl-1,3,5-benzenetriol | 153.0620 | 1.6932 |
| Scopine: 3-Ketone | 154.0876 | 0.9011 |
| 3,4-Dihydroxybenzyl alcohol 4-Me ether | 155.0640 | 1.4218 |
| 4-Amino-2-hydoxy-5- | 156.0839 | 0.9029 |
| (hydroxymethyl)pyrimidine; OH-form: | | |
| 2-Me ether | | |
| 5-Hydroxy-3-methoxy-7- | 157.0494 | 13.5094 |
| oxabicyclo[4.1.0]hept-3-en-2-one | | |
| 4-Quinolinecarboxaldehyde | 158.0649 | 1.6293 |
| 2-Amino-4-methylenepentanedioic acid | 159.0675 | 1.1446 |
| Amide | | |
| 4-Hydroxy-1,1-dimethylpyrrolidinium-2- | 160.0910 | 0.6052 |
| carboxylate | | |
| N-Benzoylglycine: Nitrile | 161.0622 | 2.0087 |
| 3-Aminotetrahydro-5-(hydroxymethyl)- | 162.0746 | 1.9322 |
| 3-furancarboxylic acid | | |
| 1-Methylpropyl 1-propenyl disulfide | 163.0628 | 13.0231 |
| N-[2-(4- | 164.0694 | 1.2294 |
| Hydroxyphenyl)ethenyl]formamide | | |
| Rhamnose | 165.0714 | 4.6017 |
| 2-Amino-2-phenylpropanoic acid | 166.0832 | 0.9314 |
| Amino-1,4-benzoquinone: N-Me, 4- | 167.0872 | 1.6428 |
| methyloxime | | |
| 2′,4′-Dihydroxyacetophenone Oxime | 168.0760 | 1.0802 |
| 1-(2,4-Dihydroxyphenyl)-2-propanol | 169.0849 | 4.8552 |
| 2-Amino-3-(3-furanyl)propanoic acid N- | 170.0831 | 3.5677 |
| Me | | |
| 1-(3,4-Dihydroxyphenyl)-1,2-ethanediol | 171.0740 | 1.5565 |
| 2-Propylquinoline | 172.1130 | 0.8784 |
| Clazamycin | 173.0476 | 2.7776 |
| 2-Amino-4-ethylidenepentanedioic acid | 174.0759 | 1.5013 |
| 4,5,6-Trihydroxy-6-(hydroxymethyl)-2- | 175.0574 | 11.0468 |
| cyclohexen-1-one | | |
| 1H-Indole-2,3-dione: N-Et | 176.0704 | 1.2468 |
| 2-Amino-3-(oxalylamino)propanoic acid | 177.0572 | 4.3854 |
| N-[2-(4- | 178.0806 | 1.3371 |
| Hydroxyphenyl)ethenyl]formamide: Me | | |
| ether | | |
| N-Benzoylglycine: Amide | 179.0748 | 2.1542 |
| 6-Deoxymannonic acid Amide | 180.0885 | 5.0404 |
| Theophylline | 181.0749 | 4.7380 |
| 2-Amino-3-(2-hydroxyphenyl)propanoic | 182.0843 | 3.1874 |
| acid | | |
| 2,5-Furandiacetic acid: Diamide | 183.0861 | 2.1272 |
| Pyridoxine 5-Me ether | 184.0938 | 0.9187 |
| 4,6-Dimethyl-1,2,3,5-benzenetetrol 1- | 185.0769 | 1.2797 |
| Me ether | | |
| 4-(1H-Indol-3-yl)-3-buten-2-one | 186.0872 | 0.7624 |
| 4-Hydroxy-3-(3-methyl-3-buten-1- | 187.0713 | 3.5093 |
| ynyl)benzoic acid: 1′-Aldehyde | | |
| 2-Amino-4-propylidenepentanedioic | 188.0919 | 1.8444 |
| acid | | |
| 5-Hydroxy-7-methyl-1,4- | 189.0576 | 4.6903 |
| naphthoquinone | | |
| Glycylglycylglycine | 190.0908 | 1.7468 |
| Khusitene | 191.1793 | 7.5229 |
| Riburonic acid; β-D-Furanose-form: Me | 193.0704 | 57.3064 |
| glycoside, Me ester | | |
| 2-Hydroxy-2H-1,4-benzoxazin-3(4H)- | 194.0792 | 6.0491 |
| one; (R)-form: Me ether, N-Me | | |
| 3,4-Dihydro-3,8-dihydroxy-3-methyl-1H- | 195.0749 | 4.1598 |
| 2-benzopyran-1-one | | |
| 2-Amino-3-(4- | 196.0996 | 2.0202 |
| hydroxymethylphenyl)propanoic acid | | |
| 2,3-Dihydroxy-3-phenylpropanoicacid | 197.0830 | 2.7234 |
| (2RS,3RS)-form: Me ester | | |
| Stizolamine | 198.1002 | 6.2518 |
| 3,5,7,8-Tridecatetraene-10,12-diynoic | 199.0850 | 3.1205 |
| acid | | |
| Anticapsin | 200.0979 | 0.9593 |
| Allantoin 1-Ac | 201.0659 | 1.8624 |
| 3-(1H-Indol-3-yl)-2-propenoic acid Me | 202.0965 | 0.6056 |
| ester | | |
| 3-Hydroxy-5-methyl-1- | 203.0648 | 2.9293 |
| naphthalenecarboxylic acid | | |
| Glycylglycylglycine: Me ester | 204.0905 | 1.6292 |
| 1H-Indole-2,3-dione: 3-Semicarbazone | 205.0692 | 6.8509 |
| 3-Aminodihydro-2(3H)-furanone N- | 206.0829 | 2.1770 |
| Benzoyl | | |
| Murrayacarpin A | 207.0708 | 8.7905 |
| Felinine | 208.1066 | 1.9962 |
| Xanthostemone | 209.1214 | 2.1669 |
| Carbazole: Hydrazone | 210.0941 | 5.1782 |
| 3-Acetyl-2,4,6-trihydroxybenzaldehyde: | 211.0700 | 4.1145 |
| 4-Me ether | | |
| 2-Amino-3-(2,4-dihydroxy-6- | 212.0938 | 10.7599 |
| methylphenyl)propanoic acid | | |
| 3,5-Dihydroxystilbene | 213.1009 | 3.4161 |
| 9H-Pyrido[3,4-b]indol-6-ol: Me ether, N2- | 214.1104 | 2.4620 |
| Me | | |
| 2-Hydroxybenzoic acid: Ph ester | 215.0742 | 1.7411 |
| 3-Hydroxy-5-methyl-1- | 216.0939 | 0.7488 |
| naphthalenecarboxylic acid: Me ether, | | |
| amide | | |
| Uracil: 1-Benzoyl | 217.0546 | 8.9856 |
| 1H-Indol-3-ol; OH-form: 1,3-Di-Ac | 218.0797 | 2.6620 |
| 3,4,5-Trihydroxy-1,2- | 219.0697 | 6.1475 |
| benzenedicarboxylic acid: Tri-Me ether, | | |
| dinitrile | | |
| 2-Amino-4-hydroxy-4-(2- | 220.1131 | 2.6933 |
| methylpropyl)pentanedioic acid | | |
| Cyclo(glycyltyrosyl) | 221.0947 | 2.4560 |
| Isovalthine | 222.0838 | 0.8183 |
| 6,7,9-Trihydroxy-3-methylcyclohepta[c]pyran- | 223.0659 | 9.1617 |
| 8(1H)-one | | |
| 1,2,3,4-Tetrahydro-6,7-dihydroxy-1- | 224.0885 | 2.0644 |
| methyl-3-isoquinolinecarboxylic acid | | |
| 1,2,3-Benzenetriol: 1-Me ether, 2,4-di- | 225.0771 | 6.4369 |
| Ac | | |
| 2-Amino-4-hydroxy-4-(4- | 226.0992 | 1.9173 |
| hydroxyphenyl)-3-methylbutanoic acid | | |
| Carnosine | 227.1066 | 1.6102 |
| 2′-Deoxycytidine | 228.0929 | 0.5939 |
| 2-(2-Hydroxybutyl)-6-(2-hydroxypropyl)- | 228.1978 | 2.0806 |
| 1-methylpiperidine: 3,4-Didehydro | | |
| 2-Hydroxybenzoic acid: Benzyl ester | 229.0810 | 4.4424 |
| Caerulomycin | 230.1013 | 2.0490 |
| 2-Amino-3-tetradecanol | 230.2515 | 0.8065 |
| 3-Hydroxy-5-methyl-1- | 231.0998 | 1.9138 |
| naphthalenecarboxylic acid: Et ester | | |
| Coryneine: Chloride | 232.1136 | 1.4175 |
| 4,5,6-Trihydroxy-2- | 233.0714 | 2.6234 |
| naphthalenecarboxaldehyde: 4,5-Di-Me | | |
| ether | | |
| 1,2,3,4-Tetrahydro-4-oxo-2- | 234.0803 | 2.3573 |
| quinolinecarboxylic acid N-Ac | | |
| 2,5,7-Trihydroxy-1,4-naphthoquinone | 235.0631 | 22.1677 |
| 2,7-Di-Me ether | | |
| 2-Amino-2-deoxygalacturonic acid N-Ac | 236.0800 | 4.0517 |
| Murrayacarpin A: 5-Methoxy | 237.0817 | 4.2714 |
| Eritadenine; (2R,3R)-form: 3-Deoxy | 238.0868 | 1.2908 |
| Glutamine N5-(4-Hydroxyphenyl) | 239.1122 | 2.1055 |
| 9H-Carbazole-3-carboxylicacid: Et ester | 240.1073 | 1.2561 |
| Thevefolic acid B: 1-Me ester | 241.0756 | 6.0123 |
| 7-Hydroxy-β-carboline-1-carboxylicacid: | 242.0957 | 2.0068 |
| Me ether, amide | | |
| Theophylline: N7-(2-Chloroethyl) | 243.0616 | 13.6296 |
| Vertilecanine A; (R)-form: Me ester | 244.0925 | 2.6473 |
| 2,4,6-Trihydroxy-3- | 245.0875 | 2.3213 |
| methylbenzophenone | | |
| Mycosporin-Gly | 246.1050 | 1.0162 |
| 4,5-Dihydroxy-2,6-octadienoicacid: 2,3- | 247.1188 | 5.2056 |
| Dihydroxypropyl ester | | |
| Evernitrose; L-Pyranose-form: Ac | 248.1172 | 1.3495 |
| 2,5,7-Trihydroxy-1,4-naphthoquinone | 249.0840 | 3.4962 |
| Tri-Me ether | | |
| 1,5,6,7-Isoquinolinetetrol; NH-form: Tri- | 250.1162 | 1.2172 |
| Me ether, N-Me | | |
| N-Glutamylcysteine | 251.0684 | 1.7238 |
| Aspartic acid N-Phenylacetyl | 252.0881 | 2.5311 |
| 1,2,3-Benzenetriol: Tri-Ac | 253.0719 | 30.5243 |
| Ichthyopterin | 254.0808 | 4.5560 |
| Phosphoarginine | 255.0832 | 5.6451 |
| 1,3-Dihydroxyacridone: 3-Me ether, N- | 256.0983 | 2.0607 |
| Me | | |
| 3,4,5-Trihydroxy-1,2- | 257.0756 | 3.0996 |
| benzenedicarboxylic acid: Tri-Me ether | | |
| Mycosporin-Gly; (S)-form: Me ester | 258.0959 | 0.8403 |
| Uridine: N-Me | 259.0861 | 5.7013 |
| Furo[2,3-b]quinoline-4,7,8-triol; OH- | 260.0938 | 2.0800 |
| form: Tri-Me ether | | |
| Obliquin; (S)-form: 5′-Hydroxy | 261.0691 | 10.1977 |
| 1,5-Dihydro-5-hydroxy-2H-pyrrol-2-one | 262.0962 | 1.8776 |
| O-β-D-Glucopyranoside | | |
| Furodysinin 14-(Methylthio) | 263.1470 | 2.1658 |
| 2-Pyrrolidineacetic acid N- | 264.1157 | 0.8183 |
| Benzyloxycarbonyl | | |
| muco-Inositol 3,6-Di-Ac | 265.0927 | 2.4011 |
| Roemerine N-De-Me | 266.1232 | 1.0552 |
| 2,3-Dihydroxy-3-phenylpropanoicacid | 267.0894 | 5.4766 |
| Di-Ac | | |
| 2′-Deoxyribofuranosylguanine | 268.0989 | 1.5507 |
| 6-Hydroxy-1-phenazinecarboxylic acid: | 269.0868 | 1.9918 |
| Me ether, Me ester | | |
| 1-(2-Carboxyanilino)-1-deoxyribulose | 270.1000 | 1.7063 |
| 4-Hydroxy-N,N-dimethyltryptamine: N- | 271.0834 | 35.6577 |
| De-Me,O-phosphate | | |
| 2-Hydroxybenzoic acid: 4- | 272.0944 | 6.4933 |
| (Acetylamino)phenyl ester | | |
| 1,6-Phenazinediol: Di-Me ether, 5,10- | 273.0925 | 4.4957 |
| dioxide | | |
| Furo[2,3-b]quinoline-4,7,8-triol; NH- | 274.1035 | 1.0796 |
| form: 7,8-Di-Me ether, N-Et | | |
| Diethanolamine: N-Dodecyl | 274.2784 | 1.0868 |
| arabino-2-Hexulosonic acid; D-form: | 275.1045 | 2.2117 |
| 3,4:5,6-Di-O-isopropylidene | | |
| 4-Hydroxy-2-(hydroxymethyl)-2- | 276.1079 | 1.0628 |
| butenoicacid Nitrile,4-O-β-D- | | |
| glucopyranoside | | |
| 8-Hydroxy-1(10),4,11(13)- | 277.1061 | 2.4246 |
| germacratriene-12,6:14,2-diolide | | |
| Lycomarasmine | 278.1052 | 1.2016 |
| Aspergillomarasmine B | 279.0764 | 8.3105 |
| Angustmycin A | 280.0954 | 2.0174 |
| 9,12-Octadecadienoic acid | 281.2433 | 2.0672 |
| Bharatamine | 282.1504 | 0.6005 |
| 10-Octadecenoic acid Amide | 282.2780 | 0.8848 |
| Mycorrhizinol | 283.0836 | 7.7028 |
| 6,7-Dihydroxy-4-methyl-5H-indeno[1,2- | 284.0996 | 1.4088 |
| b]pyridin-5-one: 6-Me ether, Ac | | |
| 3,4,5-Trihydroxy-1,2- | 285.0911 | 5.0402 |
| benzenedicarboxylic acid: Tri-Me ether, | | |
| di-Me ester | | |
| 2-Amino-3-(3-hydroxyphenyl)propanoic | 286.1123 | 0.7342 |
| acid N-Benzoyl | | |
| 2-Oxohexadecanoic acid: Oxime | 286.2454 | 0.2474 |
| 2-Amino-3-octadecanol | 286.3040 | 0.1129 |
| Claussequinone | 287.0841 | 1.8673 |
| Rutaecarpine | 288.1115 | 2.2261 |
| 1,6-Anhydromannose; β-D-Pyranose- | 289.0948 | 24.3084 |
| form: Tri-Ac | | |
| N-(3-Hydroxy-1-oxocyclopent-2-en-2-yl)- | 290.1021 | 4.2782 |
| 3-(4-hydroxy-3-methoxyphenyl) | | |
| Erythritol Tetra-Ac | 291.1050 | 2.7706 |
| Mescaline succinimide: 3,4-Didehydro | 292.1134 | 1.2239 |
| Ecklonialactone A: 6,7-Dihydro | 293.2069 | 1.6622 |
| Mescaline succinimide | 294.1258 | 0.4156 |
| 16-Hydroxy-9,12,14-octadecatrienoic | 295.2292 | 2.3458 |
| acid | | |
| Pyridoxine Tri-Ac | 296.1108 | 1.4901 |
| 13-Oxo-9-octadecenoic acid | 297.2488 | 2.2620 |
| Gindaricine | 298.1429 | 0.4890 |
| Cassine | 298.2685 | 0.5779 |
| 4-Oxooctadecanoic acid | 299.2614 | 2.6964 |
| Salutaridine: O6-De-Me, N-de-Me | 300.1196 | 0.3965 |
| 3-Isocyano-3,7,11,15-tetramethyl- | 300.2715 | 0.4500 |
| 1,6,10,14-hexadecatetraene | | |
| 2-Hydroxybenzoic acid: O-β-D- | 301.0930 | 13.3924 |
| Glucopyranoside | | |
| 1,2,3,5-Tetrahydroxyacridone: 2,3-Di- | 302.1034 | 2.9726 |
| Me ether, N-Me | | |
| Antibiotic BE 10988 | 303.0610 | 10.1504 |
| Benzamide: 4-Methoxy, 2′,4′-dihydroxy | 304.0857 | 2.5038 |
| 6-Deoxytalose; a-L-Pyranose-form: Me | 305.1189 | 1.5077 |
| glycoside, tri-Ac | | |
| Peepuloidine | 306.1291 | 1.9227 |
| 2-(1,4-Dihydroxy-4-methylpentyl)-5,8- | 307.1091 | 8.8528 |
| dihydroxy-1,4-naphthoquinone | | |
| Aspergillomarasmine A | 308.1180 | 1.9497 |
| Indicaxanthin | 309.1051 | 4.2895 |
| Dendrobates Alkaloid 309B | 310.3095 | 2.0622 |
| Lachnelluloic acid | 311.2211 | 1.8894 |
| Lysergic acid a-Hydroxyethylamide | 312.1709 | 1.2974 |
| Conkurchine | 313.2741 | 5.4268 |
| Angustine | 314.1289 | 2.8492 |
| 3,4,9-Trimethoxypterocarpan | 315.1299 | 1.9997 |
| Saxitoxin: N1-Hydroxy | 316.1422 | 0.5642 |
| Panamine | 316.2787 | 0.5930 |
| 3,4′,5,7-Tetrahydroxy-3′- | 317.0730 | 7.2083 |
| methoxyflavone | | |
| Schumannificine | 318.1051 | 2.7047 |
| 1,4-Benzenediol: Dibenzoyl | 319.1053 | 15.4199 |
| Rutaecarpine: 7β,8a-Dihydroxy | 320.1122 | 3.0453 |
| Methyl β-D-glucopyranoside: 2,3,4-Tri- | 321.1122 | 3.8583 |
| Ac | | |
| Colletochlorin A Dechloro | 323.1799 | 1.1946 |
| Bilanafos | 324.1338 | 0.6355 |
| Stravidin S3 | 324.2356 | 0.2163 |
| 2-Aminoethanol: N-(9Z,12Z- | 324.2932 | 0.4507 |
| Octadecadienoyl) | | |
| Agarobiose | 325.1191 | 4.5854 |
| Tetrahydrothalifendine | 326.1301 | 0.6614 |
| 1,2-Dihydroxy-16-heptadecen-4-one: 1- | 327.2549 | 2.5624 |
| Ac | | |
| 2-Acetamido-2-deoxyglucose; D-form: | 328.1332 | 0.3474 |
| Di-Et dithioacetal | | |
| 2-Dodecyl-3-methyl butanedioicacid (2R, | 329.2638 | 1.6311 |
| 3S)-form: Di-Me ester | | |
| 2,3,7,8,10,11-Hexahydroxy-4-guaien- | 331.1303 | 2.7260 |
| 12,6-olide | | |
| 2-Amino-2,3-dideoxy-ribo-hexose; a-D- | 332.1317 | 0.9008 |
| Pyranose-form: N,1,4,6-Tetra-Ac | | |
| 1,5-Anhydromannitol; D-form: Tetra-Ac | 333.1165 | 2.3585 |
| 2-Amino-2-deoxyglucose; β-D-Pyranose- | 334.1529 | 0.4146 |
| form: Et glycoside, 3,4,6-tri-Ac | | |
| a-Amino-2,5-dihydro-5-oxo-4- | 335.1018 | 1.6856 |
| isoxazolepropanoic acid; (S)-form: N2- | | |
| β-D-Glucosyl | | |
| Duguenaine: 11-Methoxy | 336.1227 | 0.2345 |
| 2,4-Octadecadienoic acid; (2E,4E)- | 336.3258 | 1.3828 |
| form: 2-Methylpropylamide | | |
| Glabrone | 337.1163 | 3.8085 |
| 13-Docosenoic acid; (Z)-form: Amide | 338.3413 | 15.8305 |
| 1,2-Dihydroxy-5-heneicosen-4-one | 341.3062 | 1.3734 |
| Sorbistin D | 342.1875 | 0.8267 |
| Pseudocordatolide C | 343.1566 | 2.1430 |
| Cataline: O1-De-Me,N-de-Me | 344.1490 | 0.4398 |
| Carnitine,INN O-Dodecanoyl | 344.2810 | 0.6123 |
| Boviquinone 3 | 345.2069 | 1.4053 |
| 3,3′,4′,5,7,8-Hexahydroxyflavone: 3,8- | 347.0845 | 3.8002 |
| Di-Me ether | | |
| Phosphoenolpyruvic acid: Amide, P,P- | 348.1089 | 0.7746 |
| dibenzyl ester | | |
| 3,3′,4,4′,9,9′-Hexahydroxy-7,7′- | 349.1237 | 1.7255 |
| epoxylignan | | |
| Erucifoline | 350.1547 | 0.5672 |
| 6-(1,3,5,7,9,11,15-Heptadecaheptaenyl)- | 351.1942 | 1.7972 |
| 4-methoxy-2H-pyran-2-one; (all-E)- | | |
| form | | |
| 12-Oxooctadecanoic acid: Pyrrolidide | 352.3260 | 1.4069 |
| Elliptone | 353.1106 | 0.1963 |
| Tornabeatin C | 353.3109 | 1.1065 |
| Hackelidine: 7-Ac | 354.1532 | 0.4737 |
| 2-Amino-4,8-docosadiene-1,3-diol | 354.3381 | 3.4971 |
| 14-Hydroxycarda-3,5,20(22)-trienolide | 355.2196 | 3.5200 |
| Rutacridone: 1′,2′-Dihydro, 1′-hydroxy, | 356.1501 | 0.6544 |
| 2′-methoxy | | |
| 2-Methylaminoacetic acid: N- | 356.3177 | 0.9202 |
| Octadecanoyl | | |
| Estra-1,3,5(10)-triene-3,17-diol Di-Ac | 357.2134 | 1.3697 |
| 12-Hydroxy-25-nor-17-scalaren-24-al | 359.2861 | 1.0081 |
| 3-(3,4-Dihydroxyphenyl)-2- | 361.1197 | 2.0793 |
| hydroxypropanoicacid 4′-O-β-D- | | |
| Glucopyranoside | | |
| 1,2,3,4,5,6-Hexahydroxyacridone: | 362.1302 | 0.5669 |
| 2,3,4,5-Tetra-Me ether, N-Me | | |
| Tagatose; a-D-Pyranose-form: Me | 363.1225 | 0.3848 |
| glycoside, tetra-Ac | | |
| Tetraethylene glycol Monododecyl | 363.3170 | 0.5427 |
| ether | | |
| 13(16),14-Labdadiene-3,6,8-triol 6-Ac | 365.2704 | 0.5508 |
| 15-Tetracosenoic acid Amide | 366.3686 | 0.7524 |
| Tetrahydro-2-furanmethanol 9Z- | 367.3236 | 2.2478 |
| Octadecenoyl | | |
| Mescaline citrimide | 368.1393 | 0.3660 |
| 1(10),4-Germacradien-6-ol 4- | 369.2508 | 4.2115 |
| Hydroxycinnamoyl | | |
| Aplidiasphingosine | 370.3360 | 1.4092 |
| 2′,4′,5,5′,7-Pentahydroxy-6- | 371.1073 | 0.8604 |
| prenylisoflavone | | |
| 4-(2-Amino-3-hydroxyphenyl)-4- | 372.1232 | 1.1713 |
| oxobutanoic acid: O-β-D- | | |
| Glucopyranoside | | |
| Capaurine | 373.1825 | 1.2664 |
| 2,3,3′,4,4′-Pentahydroxylignan-9,9′- | 375.1527 | 0.1567 |
| olide 3,3′-Di-Me ether | | |
| 3-Hydroxychol-11-en-24-oicacid; | 375.2803 | 0.0453 |
| (3a,5β)-form | | |
| 10′Apo-β-caroten-10′-ol: 10′-Aldehyde | 377.2820 | 0.8902 |
| β-Sorigenin: 8-O-β-D-Glucopyranoside | 379.1105 | 2.2602 |
| Karnamicin C3: 4″-Ketone | 380.1305 | 0.3916 |
| Psylloborine A | 381.3172 | 1.2545 |
| 3-O-Caffeoylquinicacid 3′-Me ether, Me | 383.1318 | 0.4863 |
| ester | | |
| Ergosta-7,22-diene | 383.3693 | 3.7260 |
| Sesangolin | 385.1320 | 2.2538 |
| Glaucamine: 8-Epimer | 386.1702 | 0.5381 |
| 5-Alkyl-1H-pyrrole-2-carboxaldehydes; | 386.3392 | 0.3710 |
| 5-(12Z,15Z-Heneicosadienyl)-1H- | | |
| pyrrole-2-carboxaldehyde | | |
| Eudesmin | 387.1734 | 1.5715 |
| Buxus Alkaloid B387 | 388.3135 | 0.5917 |
| Samandinine | 390.3081 | 0.3544 |
| myo-Inositol 1,2,3,4,6-Penta-Ac | 391.1273 | 0.3961 |
| 13(24),17-Cheilanthadiene-1,6,19-triol | 391.3260 | 1.3162 |
| Dictyolucidine: N-Ac | 392.3262 | 0.2066 |
| 2-Amino-3-(3,4- | 393.1229 | 0.1689 |
| dihydroxyphenyl)propanoic acid; (S)- | | |
| form: 5,5′-Dimer | | |
| Phloeodictyne A; Phloeodictyne 5,4i | 394.3576 | 0.9663 |
| 24-Nor-4(23),9(11)-fernadiene | 395.3726 | 2.9781 |
| 24-Nor-12-ursene | 397.3835 | 28.9439 |
| Cholest-4-en-3-one: E-Oxime | 400.3528 | 1.2570 |
| 3-Hydroxyandrost-5-en-17-one; 3β- | 401.3099 | 2.6969 |
| form: 3-Heptanoyl | | |
| 20-Aminopregn-5-en-3-ol O,N-Di-Ac | 402.3064 | 0.6272 |
| Cyanobacterin B | 403.1288 | 0.2705 |
| Lemuninol A | 405.1376 | 0.4865 |
| 3-Hydroxy-6-oxocholan-24-oic acid Me | 405.3011 | 0.8784 |
| ester | | |
| 3,18,20-Filicatriene | 407.3596 | 2.0311 |
| 2-Methyl-2,6-eicosadienoic acid (2- | 408.3522 | 0.8823 |
| Acetoxyethyl)amide | | |
| 3-Glucosyl-2,4,4′,6- | 409.1191 | 0.8131 |
| tetrahydroxybenzophenone | | |
| 11,13(18)-Oleanadiene | 409.3859 | 11.2891 |
| 15-Chloro-3,4,8-trihydroxy- | 411.1187 | 0.3375 |
| 10(14),11(13)-guaiadien-12,6-olide 8-O- | | |
| (2-Methyl-4-oxo-2E-butenoyl) | | |
| Stigmasta-5,7,24(28)-trien-3-ol | 411.3692 | 9.9840 |
| Pancratistatin 1-O-(3-Hydroxybutanoyl) | 412.1290 | 0.2709 |
| 14-Methyl-9,19-cycloergost-24(28)-en- | 413.3819 | 3.7035 |
| 3-ol | | |
| Excelsin‡ | 415.1398 | 3.0916 |
| Krigeine: 7-Ketone, O-de-Me, di-Ac | 416.1299 | 0.3200 |
| 3-Hydroxycholest-5-en-7-one; 3β-form: | 416.3605 | 1.4064 |
| E-Oxime | | |
| 1-O-Coumaroylglycerol 3′-Hydroxy, 2- | 417.1401 | 0.4447 |
| O-β-D-glucopyranoside | | |
| Axinellamine B | 419.3382 | 0.6081 |
| Hipposterol | 421.3638 | 1.1415 |
| 17-Oxo-20-hexacosenoic acid Me ester | 423.3796 | 2.2079 |
| Semiplenamide D | 424.3886 | 0.9200 |
| 3,5-Dioxohexacosanoicacid | 425.3680 | 3.1330 |
| 4-Methyl-15-azasterol | 426.3805 | 1.7297 |
| 21-Hydroxy-30-nor-20(29)-friedelen-3- | 427.3675 | 3.7782 |
| one | | |
| N-(1-Hydroxymethyl-2-methoxyethyl)-7- | 428.3785 | 1.0595 |
| methoxy-4-eicosenamide | | |
| Cholest-22-en-3-ol Ac | 429.3785 | 3.1737 |
| Cholest-4-ene-3,6-diol Di-Me ether | 431.3921 | 1.6361 |
| myo-Inositol Hexa-Ac | 433.1371 | 1.3269 |
| 8,11′;12,12′-Bi[1(10),7-errmophiladien- | 433.3170 | 0.1597 |
| 9-one] | | |
| Solacapine | 433.3725 | 0.5790 |
| Furo[2,3-b]quinoline-4,7,8-triol; OH- | 434.1530 | 0.3161 |
| form: 4,8-Di-Me ether, 7-O-(O-acetyl-a- | | |
| L-rhamnopyranoside) | | |
| 2′,3′,4′,5,5′,6,7,8-Octahydroxyflavone: | 435.1316 | 0.2682 |
| 2′,4′,5′,6,7,8-Hexa-Me ether | | |
| Cholestane-3,7,12,23-tetrol | 437.3610 | 1.1998 |
| 22,25-Epoxylanosta-7,9(11)-dien-3-one | 439.3622 | 21.7851 |
| Procevine: O-Ac | 440.3626 | 8.1077 |
| 3-Hydroxylanosta-9(11),24-dien-23-one | 441.3700 | 3.5470 |
| Stigmast-4-ene-3,6-diol 3,6-Diketone, 6- | 442.3655 | 0.9833 |
| oxime | | |
| Cyclovirobuxeinel: N3,N3,N20-Tri- | 443.3735 | 0.9388 |
| Me,N20-formyl | | |
| Methyl 3-alkylpyrrole-2-carboxylates; | 444.3793 | 0.3938 |
| Methyl 3-tricosyl-1H-pyrrole-2- | | |
| carboxylate: Tetradehydro | | |
| Stigmast-5-ene-3,7,22-triol 7-Ketone | 445.3716 | 1.8010 |
| Pseurotins; Pseurotin E | 446.1511 | 0.2506 |
| CephamycinC | 447.1158 | 0.0912 |
| 1-Nonadecene-4,6,8,10,12,14-Hexa-Me | 447.3618 | 1.0754 |
| ether | | |
| 6-Tricosyl-1,2,4-benzenetriol | 449.3909 | 0.8655 |
| Trifochalcanoloside I | 451.1528 | 1.6607 |
| Cholestane-3,7,12,25,26-pentol | 453.3639 | 1.4251 |
| Mycalazoles; Mycalazole 1: 7′,8′,10′,11′,- | 454.3593 | 0.5220 |
| Tetrahydro | | |
| 3-Hydroxy-12-oleanen-27-oic acid; 3a- | 455.3617 | 1.3870 |
| form: 3-Ketone | | |
| Isorubijervine: 18-Ac | 456.3536 | 0.2596 |
| 12-Hydroxy-3,7-friedelanedione | 457.3760 | 2.9234 |
| Stellettazole A | 459.3736 | 0.9642 |
| 3,20-Diaminopregnane-2,4-diol N20,N20- | 461.3783 | 0.8493 |
| Di-Me, N3-tigloyl | | |
| 2′,3′,4′,5,5′,6,7,8-Octahydroxyflavone: | 463.1603 | 0.5445 |
| Octa-Me ether | | |
| Halicyclamine A | 463.4027 | 1.0916 |
| Saraine 1: 1,2,9-Triepimer | 467.4009 | 0.7551 |
| 3,29-Dihydroxy-12-oleanen-27-oic acid; | 469.3370 | 0.4688 |
| 3a-form: 3-Ketone, 29-aldehyde | | |
| 16,28-Dihydroxy-3-oxo-30-friedelanoic | 471.3507 | 1.0512 |
| acid Lactone | | |
| 3,29-Dihydroxy-12-oleanen-27-oic acid | 473.3613 | 0.4766 |
| 20,32-Cyclobishomo-20,22,31- | 475.4231 | 0.9629 |
| hopatriene 32-Propyl | | |
| 3,7,12-Trihydroxystigmastan-26-oic acid | 479.3742 | 0.2283 |
| 25,28-Dimethylstigmasta-5,22,28-trien- | 481.4014 | 0.6004 |
| 3-ol Ac | | |
| 17,28-Dotriacontadiene-2,4,31-triyne- | 483.3835 | 0.4223 |
| 1,6,30-triol | | |
| 3,4-Secocycloarta-4(28),24-diene-3,26- | 485.3629 | 1.0342 |
| dioic acid 3-Me ester | | |
| Myrianthine C | 487.3254 | 0.1504 |
| Cholest-5-ene-3,7-diol Di-Ac | 487.3717 | 0.3770 |
| 24-Tritriacontene-2,4-dione | 491.4903 | 1.3880 |
| 12-Oxotritriacontanal | 493.5017 | 7.1312 |
| C36 Botryococcene | 495.4926 | 1.3662 |
| Cycloprotobuxine I: 6,7-Didehydro, N3, | 503.4088 | 0.6141 |
| N20,N20-tri-Me,N3-benzoyl | | |
| 30-Methyl-28-oxo-29-dotriacontenoic | 507.4817 | 1.0340 |
| acid | | |
| Nodolidol: Ac | 509.4596 | 0.4120 |
| Plakinamine C | 511.4300 | 0.5247 |
| Holost-8-ene-3,23-diol 23-Ac | 515.3787 | 0.3979 |
| 5,14: 7,8-Diepoxy-5-marasmanol | 517.4237 | 0.4810 |
| Octadecanoyl | | |
| Hoprominol | 523.4628 | 0.8266 |
| Buxidienine I: 16-Deoxy, N3,N20,N20- | 529.4357 | 1.0266 |
| tri-Me, N3-(2R-hydroxy-3?- | | |
| methylpentanoyl) | | |
| 1,2-Benzenedicarboxylic acid: Ditridecyl | 531.4322 | 0.8562 |
| ester | | |
| 5,20-Dipropyl-1,16-dioxa-4,19- | 533.4393 | 0.5601 |
| diazacyclotriaconta-7,10,22,25- | | |
| tetraene-15,30-dione: 7,8-Dihydro | | |
| 2-Amino-4,8-octadecadiene-1,3-diol N- | 536.5012 | 0.6376 |
| Hexadecanoyl | | |
| 2-Amino-4-octadecene-1,3-diol N- | 538.5220 | 0.8842 |
| Hexadecanoyl | | |
| 20(29)-Lupen-3-ol; 3β: Heptanoyl | 539.4834 | 0.6182 |
| Cadabalone | 549.4866 | 0.3309 |
| 3-Methyl-3-buten-1-ol: Dotriacontanoyl | 549.5515 | 0.1236 |
| Artemoin A | 551.4990 | 0.6567 |
| 2-Amino-9-methyl-4,8-octadecadiene- | 552.5043 | 0.2901 |
| 1,3-diol N-(2R-Hydroxypentadecanoyl) | | |
| 12-Ursen-3-ol; 3β-form: Octanoyl | 553.4928 | 0.4553 |
| Glycerol 2-heptadecanoate 1- | 555.5018 | 0.5443 |
| tetradecanoate | | |
| 20(29)-Lupen-3-ol; 3β: 3- | 559.4506 | 0.5424 |
| Phenylpropanoyl | | |
| Coriacyclodienin | 573.4815 | 0.6016 |
| Montecristin | 575.5081 | 4.3382 |
| Cohibin C | 577.5192 | 1.7037 |
| Tonkinelin | 579.5260 | 0.4220 |
| 2-Phyten-1-ol 5,8,11,14,17- | 581.5246 | 0.5350 |
| Eicosapentaenoyl(all-Z) | | |
| Pyrinadine A | 589.4944 | 0.5287 |
| 2-[14-[3-(1,5- | 591.5125 | 1.4081 |
| Dimethylhexyl)cyclopentyl]-3,7,11- | | |
| trimethyltetradecyl]-3-methyl-1,4- | | |
| naphthoquinone | | |
| Uvariamicin IV | 593.5115 | 0.6179 |
| Minalemines; Minalemine A | 597.4959 | 0.6701 |
| 5,7-Dihydroxy-6-methyl-2-nonacosyl-4H- | 599.5047 | 1.7581 |
| 1-benzopyran-4-one | | |
| 3-(3,4-Dihydroxyphenyl)-2- | 601.5196 | 2.1230 |
| propenoicacid (E)-form: Triacontyl | | |
| ester | | |
| 3-(3,4-Dihydroxyphenyl)-1-propanol: 1- | 603.5404 | 1.5730 |
| O-Triacontanoyl | | |
| 19(10?9)-Abeo-3,4-secotirucall-4-ene- | 605.5244 | 0.5590 |
| 3,24,25-triol; (24R)-form: 4,5a-Epoxide, | | |
| 3-octanoyl | | |
| 2-Alkyl-5,7-dihydroxy-4H-1-benzopyran- | 613.5177 | 1.2077 |
| 4-ones; 2-Hentriacontyl-5,7-dihydroxy- | | |
| 4H-1-benzopyran-4-one | | |
| Glycerol 1-(9Z,12Z,15Z- | 615.5070 | 1.4793 |
| octadecatrienoate) 2-(9Z,12Z- | | |
| octadecadienoate) | | |
| Glycerol 1,2-di-(9Z,12Z- | 617.5214 | 2.6379 |
| octadecadienoate) | | |
| Glycerol 1-(9Z,12Z-octadecenoate) | 619.5288 | 0.8391 |
| 2-(9Z-octadecenoate) | | |
| 3-(3,4-Dihydroxyphenyl)-1-propanol: 1- | 631.5590 | 0.3887 |
| O-Dotriacontanoyl | | |
| 1,2-Bis-O-(3,7,11,15-tetramethyl- | 641.5935 | 0.2309 |
| 2,6,10-hexadecatrienyl)glycerol | | |
| 4-Aminotetrahydro-2-(4-tetradecenyl)- | 650.6164 | 0.4292 |
| 3-furanol N-(2R-Hydroxytricosanoyl) | | |
| Glycerol 1-eicosanoate 3- | 653.6079 | 0.2898 |
| octadecanoate | | |
| 2-Amino-6,9-heptacosadiene-1,3,5-triol | 664.6197 | 0.7134 |
| N-Pentadecanoyl | | |
| 4-Aminotetrahydro-2-tetradecyl-3- | 666.6314 | 0.9146 |
| furanol N-(2-Hydroxytetracosanoyl) | | |
| 26-Heptacosene-9,10-diol: 9-(9- | 675.6665 | 0.3845 |
| Octadecenoyl) | | |
| 24-Methylcycloart-25-en-3-ol | 679.6319 | 0.4175 |
| Hexadecanoyl | | |

TABLE 2
Summary of the identified compounds in Extract 2 as
determined by DART TOF-MS.

| | | Relative |
|---|---|---|
| | Measured | Abundance |
| Compound Name | Mass | (%) |
| furfural | 97.0267 | 0.2506 |
| 1,4-benzoquinone | 109.0283 | 26.0077 |
| 2-Hydroxypropanoic acid; Na | 113.0249 | 0.5174 |
| levulinic acid | 117.0545 | 8.0867 |
| indole | 118.0596 | 0.4328 |
| cysteine | 122.0337 | 0.2615 |
| Benzoic acid | 123.0438 | 23.0474 |
| niacin | 124.0469 | 1.216 |
| taurine | 126.0314 | 5.1427 |
| pyrogallol/phlorglucinol | 127.0387 | 100 |
| 1,3-Dicyanobenzene | 129.0457 | 0.6676 |
| malic acid | 135.0248 | 0.2313 |
| 2-Hydroxy-5-methyl-1,4-benz | 139.0406 | 0.2992 |
| kojic acid/muconic acid | 143.0316 | 0.7115 |
| 1,4-Dihydroxy-2-cyclopentene | 144.0657 | 7.3262 |
| 5-Fluoro-2,4(1H,3H)-pyrimidine | 145.0503 | 29.2617 |
| 3-Phenyloxiranecarboxylic ac | 146.0548 | 1.7763 |
| coumarin | 147.0473 | 4.4118 |
| O-Carbamoylserine | 149.0625 | 0.8058 |
| 1-methyl-3-phenylpropylamine | 150.1183 | 0.0595 |
| benzoylformic acid | 151.0444 | 0.1967 |
| 1,2-Benzisoxazole-3,6-diol | 152.0436 | 0.5992 |
| decadienal/santolina epoxide | 153.1285 | 0.2819 |
| Tetramethylammonium bromide | 154.0355 | 0.3701 |
| Benzeneacetyl chloride | 155.035 | 4.667 |
| 5-(Methoxymethyl)-2-furancarboxylic | 157.051 | 3.496 |
| acid | | |
| 2,3-Oxiranedicarboxylic acid | 161.0479 | 0.3698 |
| glyogen | 163.0603 | 63.0895 |
| phenylethyl isothiocyanate | 164.063 | 4.8651 |
| coumaric acid | 165.0565 | 5.1321 |
| 6N-Me, N1-oxide Adenine | 166.0645 | 0.4229 |
| phenyllactic acid | 167.0785 | 0.6331 |
| 2-Hydroxy-2-(3-hydroxyp | 169.0569 | 1.6176 |
| 1-Hydroxy-p-menthan-3-one. 3 | 171.1472 | 0.2633 |
| vitamin K3(menadione) | 173.0655 | 0.2905 |
| 5-Fluoro-2,4(1H,3H)-pyrimidine | 175.0607 | 7.3035 |
| berteroin | 176.0639 | 0.3628 |
| 4-methylumbelliferone | 177.064 | 0.7504 |
| 1-Amino-1-deoxyfructose | 180.0864 | 7.9332 |
| stilbene | 181.1016 | 1.5912 |
| Erbstatin;1′,2′-Dihydro | 182.0783 | 0.1141 |
| 2,3-dimethoxy-5-methylbenzoquinone | 183.0707 | 0.5129 |
| Tetrahydroactinidiolide | 183.1323 | 0.1837 |
| N-Ethylbenzenesulfonamide, 9 | 186.0675 | 0.1018 |
| 3-acetylcoumarin | 189.0635 | 1.5527 |
| Echinozolinone | 191.0892 | 0.4375 |
| alyssin | 192.0541 | 2.3774 |
| 1,4-Benzenediol;Trifluoromethyl | 193.0555 | 2.2748 |
| 2-Amino-2-deoxygalacturonic acid | 194.0574 | 0.5338 |
| ferulic acid | 195.0654 | 0.9727 |
| acridone | 196.0721 | 0.1476 |
| 2′,4′-Dihydroxy-6′-methoxy-3 | 197.0879 | 1.0377 |
| 2-Hydroxy-7-methyl-9H-carbazole | 198.0946 | 0.2783 |
| Methylecgonine | 200.1269 | 0.1572 |
| 2-(2,4-hexadiynylidene)-1,6- | 201.0955 | 0.3731 |
| Aconitic acid;Mixed Et ester | 203.0563 | 12.2237 |
| 3-acetamidocoumarin | 204.0606 | 1.0882 |
| 2,3-Epoxyplumbagin | 205.054 | 0.5802 |
| Bellendine | 206.1178 | 0.2665 |
| Echinozolinone | 207.086 | 1.0799 |
| 8-Methoxy-3-methyl-2H-1,3-be | 208.0672 | 0.0493 |
| hydrastinine | 208.1027 | 0.0896 |
| Epibatidine | 209.0936 | 0.1526 |
| 1S-Acetoxy-3-myodesertene | 211.1397 | 0.4118 |
| Naproxen;Nitrile | 212.1155 | 0.2972 |
| Duazomycin | 214.0876 | 0.413 |
| harmaline | 215.1196 | 0.3927 |
| cyclopentanemethanol, 2-nitro | 216.1315 | 0.0757 |
| 5-Hydroxygoniothalamin | 217.0939 | 0.68 |
| Glycerol triacetate | 219.0943 | 0.3153 |
| vitamin B5 | 220.1185 | 1.0181 |
| Indeno[1,2,3-ij][2,7]naphthyl | 221.0789 | 38.3927 |
| 4-Cyanobenzanilide | 223.0962 | 2.5769 |
| Isoplectrodorine | 224.0897 | 0.4831 |
| 3-hydroxy-DL-kynurenine | 225.0961 | 1.3176 |
| Arthropsatriol B | 227.1256 | 0.5006 |
| Fructose Butyl gly | 237.1432 | 0.0889 |
| Eritadenine;Deoxyeritadenine | 238.0984 | 5.6559 |
| hydroxymethylchalcone | 239.1135 | 0.8278 |
| Fructose 2-Chloroe | 243.0548 | 1.8739 |
| N-Benzoyl Amide | 244.0545 | 0.198 |
| 1-Epimer, Methyl ester Shanz | 245.1063 | 0.5994 |
| Ellipticine | 247.1282 | 1.1421 |
| 4-Epiphyllanthine | 248.135 | 0.2176 |
| methylflavone | 249.0893 | 0.8671 |
| 1(10),11(13)-Eremophiladiene | 249.1805 | 0.2044 |
| N,N′-Dimethyl-N,N′-dinitroso | 251.076 | 0.1701 |
| Eritadenine; | 254.0842 | 0.5825 |
| diprophyllin | 255.1135 | 0.3296 |
| Batrachamine | 255.2267 | 0.1263 |
| palmitic acid | 257.2525 | 0.1346 |
| heptadecanol | 257.2877 | 0.0933 |
| Methylmadugin | 259.1898 | 1.4185 |
| Pyrazofurin -- Antibiotic A | 260.0796 | 0.2446 |
| 2,6-Diamino-2,6-dideoxyidose | 263.1329 | 0.3015 |
| Eperuol | 263.2323 | 0.512 |
| 2-Acetamido-2-deoxyglucose 3 | 264.1368 | 0.3794 |
| Eccremocarpol B | 265.1365 | 0.6998 |
| Eduleine | 266.1274 | 0.2578 |
| 2-Ethoxy-2-oxoethyl methyl p | 267.0892 | 3.1848 |
| 9-octadecenal | 267.275 | 0.0882 |
| Zamene | 267.314 | 0.0794 |
| 7-deazainosine | 268.0942 | 0.5572 |
| Baeocystine | 271.0846 | 2.0149 |
| 4-Hydroxydianthramide B meth | 272.0931 | 0.3314 |
| Mirabilin A | 272.2115 | 0.0923 |
| 8-Epithienamycin | 273.0942 | 0.4611 |
| 12-Phenyldodecanoic acid. Be | 277.2139 | 0.8922 |
| octadecatrienoic acid | 279.2325 | 4.8764 |
| octadecadienoic acid | 281.2491 | 6.3553 |
| cyclohexanecarboxamide, N-de | 282.2763 | 3.6247 |
| Lactone. Oxacyclononadecan-2 | 283.2692 | 2.2777 |
| Amabiline | 284.1794 | 0.1492 |
| helicin | 285.0995 | 4.7822 |
| Plakortide Z;Et ester | 287.2296 | 0.2096 |
| Furanodictine B | 288.1383 | 0.2435 |
| Hydroxyanigorufone | 289.0954 | 4.4391 |
| catechin | 291.0927 | 0.3243 |
| 1-Octen-3-yl glucoside | 291.1816 | 0.338 |
| N-octyl-B-D-glucopyranoside | 293.2034 | 0.3206 |
| nordihydrocapsaicin | 294.2137 | 0.2093 |
| Conocandin | 295.2289 | 2.4532 |
| 6-Isocassine | 298.2753 | 0.7856 |
| 1,2-Dibenzoyl Glycerol | 301.1045 | 0.224 |
| lauric acid, 2-butoxyethyl ether | 301.2731 | 0.3996 |
| Bicyclomycin | 303.1276 | 0.9444 |
| aleuritic acid | 305.2372 | 0.3705 |
| Galactose 1, | 307.1126 | 0.6795 |
| 5,11,14-Eicosatrienoic acid | 307.2611 | 0.2759 |
| dihydrocapsaicin | 308.2309 | 0.4641 |
| bisdemethoxycurcumin | 309.1129 | 0.8179 |
| Erymelanthine; | 313.1596 | 0.0955 |
| 9,10-Epoxy-18-oxooctadecanoi | 313.2462 | 1.2325 |
| Prosophylline | 314.2668 | 0.2559 |
| 9,10-Dihydro-3-epiplakortin | 315.2539 | 0.8775 |
| pregnenolone | 317.2393 | 0.7933 |
| 6-Epiormosanine | 318.2967 | 0.1767 |
| 14,15-Epoxysclareol | 325.2798 | 0.8672 |
| 1-O-p-Coumaroylglucose | 327.1105 | 1.2817 |
| Sesbanimide B | 328.1437 | 0.2604 |
| 3′,4′,5,7-Tetrahydroxyflavan | 329.1043 | 0.9008 |
| 3′,6′-dihydroxy-2′,4′,5′-tri | 331.126 | 0.3893 |
| averionol C | 331.2845 | 1.7003 |
| Batzellaside A | 332.2851 | 0.3617 |
| Endiandric acid B | 333.1945 | 0.1532 |
| pregnanetriol | 337.2734 | 0.3619 |
| Baihuaqianhuoside | 343.1404 | 0.29 |
| 3-Acetoxy-16-methylheptadecane | 343.2852 | 0.4622 |
| 4,7′-Epoxy-3,8′-bilign-7-ene | 347.1164 | 1.2898 |
| 1,2,3,4-Eicosanetetrol | 347.3136 | 0.0972 |
| 6-furfurylaminopurine riboside | 348.1298 | 0.1848 |
| 10-gingerdiol | 353.2682 | 1.2855 |
| Plakortide H;11,12-Didehydro | 355.2851 | 3.0276 |
| 14,15-Epoxy-3-oxovincadiffor | 367.1701 | 0.2272 |
| Haliclonadiamine | 369.3204 | 0.7981 |
| Eicosanedioic acid;Di-Me ester | 371.3142 | 30.2284 |
| Emericolin B | 373.3186 | 1.1199 |
| 2,4,16-Eicosatrienoic acid | 374.3338 | 0.1663 |
| 3-Hydroxy-27-norcholesta-5,2-diene | 385.301 | 0.8478 |
| cornin/geniposide | 389.139 | 0.2328 |
| Buxidienine I | 389.3243 | 0.6479 |
| octyl phthalate | 391.2863 | 4.2992 |
| Ergosta-4,6,8(14),22-tetraene | 393.319 | 0.7235 |
| 24-Nor-18a-olean-12-ene | 397.3829 | 1.1246 |
| Spectamine A | 402.3031 | 0.3044 |
| fucosterol/sitosterone | 413.3799 | 0.3662 |
| calcitriol/sarsapogenin | 417.3308 | 0.2874 |
| maesaquinone | 419.3221 | 1.4428 |
| mogroside backbone-3H2O | 423.367 | 0.4672 |
| amyrenone/lupenone | 425.3766 | 0.8737 |
| 10,11-Epoxysqualene | 427.3882 | 0.7813 |
| Zeraconine | 445.3273 | 0.1279 |
| 30-Epibatzelladine D | 463.3811 | 0.1839 |

TABLE 3
Summary of the identified compounds in Extract 3 as
determined by DART TOF-MS.

| | | Relative |
|---|---|---|
| | Measured | Abundance |
| Compound Name | Mass | (%) |
| furfural | 97.0277 | 0.1758 |
| Farmiserina | 103.0413 | 0.5053 |
| 1,4-benzoquinone | 109.0288 | 0.8884 |
| 1,2-Benzenediol | 111.0455 | 0.5106 |
| 2-Hydroxypropanoic acid; Na | 113.0241 | 0.1338 |
| Succinic acid | 119.0373 | 0.8046 |
| L-threonine | 120.0563 | 0.8411 |
| acetophenone | 121.064 | 9.0014 |
| A-Benzaldoxime | 122.0524 | 1.4491 |
| Benzoic acid | 123.0435 | 72.2239 |
| niacin | 124.0478 | 5.2041 |
| guaiacol | 125.0512 | 0.4287 |
| 4-methyl-5-vinylthiazole | 126.0349 | 0.0592 |
| pyrogallol/phlorglucinol | 127.0395 | 3.795 |
| trans-2,2-dimethyl-3-heptene | 127.1557 | 0.214 |
| Arabinan | 133.0488 | 0.2455 |
| malic acid | 135.0321 | 0.1378 |
| homocysteine | 136.053 | 0.6219 |
| anisaldehyde | 137.0591 | 5.1098 |
| 4-Aminobenzoic acid | 138.0468 | 0.5491 |
| 2-Hydroxy-5-methyl-1,4-benz | 139.0403 | 2.2814 |
| kojic acid/muconic acid | 143.0289 | 0.2347 |
| 1,4-Dihydroxy-2-cyclopentene | 144.0596 | 0.4613 |
| 3-Phenyloxiranecarboxylic acid | 146.0561 | 0.554 |
| coumarin | 147.0442 | 34.29 |
| isatin | 148.0479 | 3.0553 |
| O-Carbamoylserine | 149.0599 | 5.0215 |
| N-Et Benzamide | 150.0916 | 1.4819 |
| arabinose | 151.0582 | 5.1861 |
| guanine | 152.0493 | 1.1065 |
| dihydroxyacetophenone | 153.0557 | 1.3436 |
| Benzeneacetyl chloride | 155.0342 | 28.77 |
| 2,3-Oxiranedicarboxylic acid | 161.0501 | 1.0729 |
| 6,7-Isoquinolinediol | 162.0646 | 0.2806 |
| glyogen | 163.0581 | 4.4355 |
| phenylethyl isothiocyanate | 164.0484 | 9.61 |
| coumaric acid | 165.0544 | 100 |
| Isophthalamic acid | 166.0593 | 9.7782 |
| Benzylthiourea | 167.064 | 1.8468 |
| Me ether, amide Zymonic acid | 172.0588 | 0.2869 |
| 4-methylumbelliferone | 177.0542 | 4.7931 |
| Entadamide A;Entadamide C | 178.0583 | 0.5974 |
| 1-Amino-1-deoxyfructose | 180.0826 | 2.5983 |
| 2-Deoxy-arabino-hexonic acid | 181.0765 | 8.7113 |
| Erbstatin;1′,2′-Dihydro | 182.0839 | 2.3263 |
| 2,3-dimethoxy-5-methylbenzoquinone | 183.0675 | 2.4076 |
| chlorothymol | 185.0795 | 0.6783 |
| N-Ethylbenzenesulfonamide | 186.0615 | 0.1178 |
| Erinapyrone C | 187.0622 | 0.0785 |
| Enteromycin carboxamide | 188.0708 | 0.0965 |
| dealanyl-alahopcin | 191.0683 | 0.1769 |
| alyssin | 192.0463 | 0.3769 |
| 1,4-Benzenediol;Trifluoromethyl | 193.0524 | 8.3617 |
| 2-Amino-2-deoxygalacturonic acid | 194.0571 | 3.3626 |
| ferulic acid | 195.0658 | 14.4709 |
| N-Acetyl 4-Amino-3-hydroxybenzene | 196.0697 | 1.8208 |
| 2-Hydroxy-7-methyl-9H-carbaz | 198.0948 | 1.1929 |
| leucenol | 199.0814 | 1.1538 |
| Edulitine | 206.0845 | 0.09 |
| citropten | 207.0694 | 3.8695 |
| Allaric acid Diamide | 209.0806 | 0.9521 |
| 1S-Acetoxy-3-myodesertene | 211.1343 | 1.0346 |
| Enicoflavine | 212.0985 | 0.3194 |
| Duazomycin | 214.0916 | 0.4986 |
| captopril(usp) | 218.0817 | 0.2556 |
| Siastatin B. Antibiotic A 7 | 219.0985 | 0.73 |
| 6,7-dimethoxy-4-methylcoumarin | 221.0834 | 0.2135 |
| 2-Methylfervenulone | 224.069 | 2.0429 |
| 3,5-dimethoxy-4-hydroxy cinnamic acid | 225.0775 | 9.8071 |
| cyclocytidine | 226.0806 | 0.9828 |
| Aspyrone;Ac | 227.1019 | 0.6248 |
| Ergothioneine; | 230.1008 | 0.1338 |
| Apiose | 231.1317 | 0.5435 |
| 3-Deoxy-manno-oct-2-ulosonic acid | 239.0839 | 0.3573 |
| 6N-Benzoyl Adenine | 240.0832 | 0.1128 |
| O-Acetyl Harmol | 241.1074 | 0.5671 |
| Begonanline | 243.0865 | 0.2401 |
| Ellipticine | 247.1301 | 0.3847 |
| methylflavone | 249.0914 | 0.9613 |
| 2-Amino-2-deoxyglucuronic acid | 250.0966 | 0.1239 |
| 4-Epilegionamic acid | 251.1262 | 0.2352 |
| 3-Deoxy-manno-oct-2-ulosonic acid | 253.0975 | 0.2444 |
| Batrachamine | 255.2323 | 0.7446 |
| Pterostilbene | 257.1159 | 0.1777 |
| palmitic acid | 257.2481 | 2.1453 |
| Lamiophlomiol C | 259.0826 | 0.0843 |
| Methylmadugin | 259.1883 | 1.2811 |
| 1,2-Diphenoxybenzene | 263.1027 | 0.195 |
| Eperuol | 263.2358 | 2.2894 |
| Eccremocarpol B | 265.1226 | 0.5436 |
| 9,12,15-octadecatrien-1-ol | 265.2487 | 0.1325 |
| 3-Amino-2,3,6-trideoxy-arabinose | 266.1366 | 0.1027 |
| Asimilobine | 268.1348 | 0.8606 |
| 1,6,9-Farnesatriene-3,5,11-triol | 269.2207 | 0.9442 |
| 16-Hydroxy-9-hexadecenoic acid | 271.2215 | 0.9849 |
| Mirabilin A | 272.2135 | 0.6411 |
| 8-Epithienamycin | 273.0963 | 1.0476 |
| Octadecatrienoic acid | 279.2312 | 10.8565 |
| 9,12-octadecadienoic acid | 281.2477 | 9.6248 |
| cyclohexanecarboxamide, N-deoxy | 282.2718 | 5.289 |
| Lactone. Oxacyclononadecan-2 | 283.2665 | 3.8747 |
| ethylpalmitate | 285.2799 | 5.7393 |
| 15,16-Epoxy-9(11)-parguerene | 287.2363 | 2.6242 |
| 4-Methoxydianthramide S | 288.0931 | 0.0981 |
| catechin | 291.0885 | 0.7749 |
| Edulinine | 292.1633 | 0.4634 |
| 6-Hydroxy-7,9-octadecadiynoic acid | 293.2136 | 2.4893 |
| nordihydrocapsaicin | 294.2141 | 0.7259 |
| 3-Epiaristoserratenine | 295.227 | 7.2055 |
| 6-Isocassine | 298.2747 | 3.7042 |
| 6-Isocarnavaline | 300.2872 | 1.2175 |
| Fastigiatin | 301.2307 | 1.1325 |
| hesperetin/hesperetin chalcone | 303.0893 | 3.2247 |
| arachidonic acid | 305.245 | 1.0019 |
| 5,11,14-Eicosatrienoic acid | 307.2608 | 0.8346 |
| Prosophylline | 314.2667 | 1.5299 |
| 9,10-Dihydro-3-epiplakortin | 315.2588 | 1.5499 |
| 5,6-Dibromotryptamine | 316.9595 | 0.0044 |
| azaleatin | 317.0758 | 5.2456 |
| petunidin | 318.082 | 1.1074 |
| 1,12-Epoxy-2,7,15-cembratriene | 321.2406 | 0.4867 |
| 1-O-p-Coumaroylglucose | 327.114 | 2.2655 |
| Homo-6-epipodopetaline | 328.2849 | 0.8814 |
| 3′,4′,5,7-Tetrahydroxyflavan | 329.1091 | 1.83 |
| Lithospermoside;5-Epimer | 330.118 | 0.1568 |
| Morusimic acid F | 330.2686 | 0.5171 |
| Batzellaside A | 332.2814 | 0.8357 |
| 10-shogaol | 333.2474 | 1.0054 |
| dihydrosanguinarine | 334.1058 | 0.5613 |
| Fasicularine | 335.2549 | 0.7101 |
| pregnanetriol | 337.2746 | 1.5503 |
| Kanagawamicin | 340.1169 | 0.0866 |
| 12-Epihapalindole H | 340.1696 | 0.0437 |
| N-Hexadecanoylhomoserine lactone | 340.2951 | 0.7514 |
| hexanoic acid, 4-hexadecyle | 341.3402 | 4.9986 |
| 3-Acetoxy-16-methylheptadecane | 343.2925 | 1.1575 |
| 12-Epifinetianine | 346.2467 | 0.7234 |
| 5,8,11,14-Eicosatetraenoic acid | 348.2934 | 0.7884 |
| lactucin-15-oxalate | 349.1008 | 0.5253 |
| tetrahydrocorticosterone | 351.2567 | 0.9286 |
| 10-gingerdiol | 353.2685 | 3.6951 |
| Plakortide H;11,12-Didehydro | 355.2859 | 5.4781 |
| 3,3′,4′,5,7-Pentahydroxyflavone | 359.119 | 1.3724 |
| Bacithrocin C | 362.2198 | 0.4154 |
| 13-Epiyosgadensonol | 363.2883 | 0.7666 |
| Haliclonadiamine | 369.3316 | 2.2658 |
| Eicosanedioic acid;Di-Me ester | 371.3132 | 30.8766 |
| Emericolin B | 373.319 | 1.7044 |
| 3,3′,4′,5,7,8-Hexahydroxyflavaone | 375.1156 | 1.6642 |
| lithocholic acid | 377.3109 | 0.7646 |
| Barrenazine B | 383.316 | 0.8603 |
| 3-Hydroxy-27-norcholesta-5,2-diene | 385.3051 | 1.2882 |
| 3,3′,4′,5,7,8-Hexahydroxyflavone | 389.1271 | 3.0407 |
| octyl phthalate | 391.2829 | 10.5657 |
| Ergosta-4,6,8(14),22-tetraene | 393.3159 | 1.726 |
| ergosterol/ergocalciferol | 397.342 | 1.1521 |
| 20-Epiverazine | 398.3381 | 0.3493 |
| 18,22-Epoxycholesta-5,20(22)-triene | 399.3253 | 0.7595 |
| 22-Isopropylchola-5,23-diene | 401.3385 | 1.2429 |
| Spectamine A | 402.3108 | 0.6495 |
| 24,25-Epoxy-16-scalarene-12, | 405.3094 | 0.6887 |
| Emeniveol | 406.3093 | 0.4228 |
| Baleabuxaline I | 407.33 | 1.3615 |
| N-Eicosanoyl, Me ester | 410.3558 | 0.447 |
| 3-Epidiosgenin | 415.3217 | 0.8469 |
| tomatidine | 416.3469 | 0.4368 |
| calcitriol/sarsapogenin | 417.3302 | 0.9438 |
| Passicapsin | 418.1643 | 0.0613 |
| maesaquinone | 419.3192 | 2.7273 |
| 20-epi-Hydroxyisoaflavinine | 422.3048 | 0.2887 |
| mogroside backbone-3H2O | 423.3609 | 0.6557 |
| amyrenone/lupenone | 425.3711 | 1.3061 |
| Myltalorione B | 435.3326 | 0.7107 |
| Ergosta-4,6,8(14),22-tetraene | 437.3538 | 0.5458 |
| Teleocidin A2 | 438.3152 | 0.3172 |
| 11,12-Epoxy-14-taraxeren-3-o | 439.3582 | 1.2416 |
| Baleabuxoxazine C | 445.3481 | 0.9921 |
| 23-Isokuroyurinidine | 446.3357 | 0.4379 |
| Na-Demethylalfileramine | 449.326 | 0.4232 |
| Ergost-22-ene-3,6,15,28-tetraene | 451.3776 | 0.5158 |
| 30-Epibatzelladine D | 463.3828 | 0.756 |
| 3-Epipachysamine H | 465.3921 | 0.269 |
| Enervosanone | 467.3614 | 0.5095 |
| Stellettasterol | 469.3588 | 0.2745 |
| Baikeidine | 474.3524 | 0.2848 |
| psychosine | 478.3364 | 0.1706 |
| Ergostan-3-ol;3-O-Sulfate | 483.3484 | 0.5874 |
| Baccatin A | 485.3558 | 0.3362 |
| hovenolactone/trevoagenin D | 489.3667 | 0.4497 |
| acetyl-boswellic acid | 497.4024 | 0.2448 |
| Majusculamide B | 504.3531 | 0.1878 |
| N-Isobutyrylbaleabuxaline F | 505.4087 | 0.5022 |
| Nb-Tetracosanoyltryptamine | 511.4556 | 0.4029 |
| 3-O-acetyl-11-hydroxy boswel | 515.3785 | 0.1699 |
| Stearoylplorantinone B | 517.4278 | 0.2504 |
| Benzoyl Spirost-5-en-3-ol | 519.3459 | 0.1025 |
| Nb-Hexacosanoyltryptamine | 539.5023 | 0.3816 |
| 5,8,11,14,17-Eicosapentaenoy | 581.5289 | 0.1822 |
| Reticulatain 2 | 593.515 | 0.0948 |

TABLE 4
Summary of the identified compounds in Extract 4 as
determined by DART TOF-MS.

| | | Relative |
|---|---|---|
| | Measured | Abundance |
| Compound Name | Mass | (%) |
| Benzoic acid | 123.0429 | 1.1203 |
| pyrogallol/phlorglucinol | 127.0389 | 0.6756 |
| leucine | 132.1036 | 0.7947 |
| 5-Fluoro-2,4(1H,3H)-pyrimidine | 145.0461 | 0.3095 |
| Benzeneacetyl chloride | 155.0344 | 3.8903 |
| 1-(5-Hydroxy-2-methylphenyl) | 179.0703 | 1.351 |
| 1-Amino-1-deoxyfructose | 180.078 | 0.252 |
| 2-Deoxy-arabino-hexonic acid | 181.067 | 0.2508 |
| 1,3-di-tert-butylbenzene | 191.1852 | 0.3288 |
| 9,10-Epoxytetrahydroedulan | 211.1706 | 0.4061 |
| lauric acid ethylester | 229.219 | 4.3435 |
| dodecylfuran | 237.2233 | 5.1502 |
| 11-hexadecyn-1-ol | 239.2399 | 3.8806 |
| 4,5-Epoxy-2-tetradecenoic acid | 241.1874 | 10.7586 |
| 12-Farnesanoic acid | 243.2378 | 3.058 |
| 4-(3,7-Dimethyl-2,6-octadien | 248.1719 | 0.4498 |
| Ellipticine;3,4-Dihydroellipticine | 249.1471 | 0.3138 |
| Batrachamine | 255.2304 | 41.3111 |
| palmitic acid | 257.2458 | 49.6553 |
| Methylmadugin | 259.1851 | 1.2395 |
| Zeagenin | 261.1467 | 0.4984 |
| androstane | 261.2548 | 1.5151 |
| Eperuol | 263.2355 | 0.7363 |
| 9,12,15-octadecatrien-1-ol | 265.2538 | 3.3738 |
| 9-octadecenal | 267.27 | 4.7066 |
| 7-hexadecenoic acid, methyl | 269.2453 | 8.1325 |
| 5-deoxykaempferol | 271.0544 | 0.1219 |
| 14-Serrulatene;Erogorgiaene | 271.2507 | 7.1127 |
| 4(20),5,15-Bifloratriene | 273.2618 | 2.6528 |
| 6-shogaol | 277.1857 | 0.9509 |
| phthalic acid, diisobutyl ester | 279.1605 | 8.4036 |
| 9,12-octadecadienoic acid | 281.2478 | 11.5902 |
| cyclohexanecarboxamide, N-de | 282.2721 | 6.1698 |
| Lactone. Oxacyclononadecan-2 | 283.2632 | 36.5588 |
| ethylpalmitate | 285.2781 | 52.9698 |
| 7-shogaol | 291.1948 | 9.7866 |
| Epijoubertinamine | 292.1974 | 1.6429 |
| N-octyl-B-D-glucopyranoside | 293.1975 | 0.5126 |
| 2-Amino-2-deoxyglucose Di-Et | 294.1979 | 0.1371 |
| Conocandin | 295.2345 | 1.2615 |
| 6-Isocassine | 298.2777 | 5.6862 |
| 6-Isocarnavaline | 300.2888 | 9.079 |
| lauric acid, 2-butoxyethyl ether | 301.2755 | 4.9554 |
| Falcatine | 302.1323 | 0.0268 |
| 3,3′,4′,5,7-Pentahydroxyflavone | 303.0524 | 1.7433 |
| 2,3-dihydrorobinetin | 305.0614 | 0.0929 |
| arachidonic acid | 305.2479 | 1.004 |
| 5,11,14-Eicosatrienoic acid | 307.2581 | 0.7344 |
| linoleic acid ethylester | 309.2759 | 1.5895 |
| 9-Eicosenoic acid;Amide | 310.3089 | 4.349 |
| 11-Eicosenoic acid | 311.2905 | 5.4592 |
| arachidic acid | 313.3055 | 17.2483 |
| 1-Chloroeicosane | 317.2888 | 13.984 |
| 16-Epiormosanine | 318.2934 | 1.6348 |
| Epifuntumidine | 320.2974 | 0.8543 |
| 1,12-Epoxy-2,7,15-cembratrie | 321.2437 | 0.396 |
| 3-Epiconamine | 329.296 | 10.1028 |
| Batzellaside A | 332.2864 | 1.7492 |
| 11,19-Eicosadien-1-ol;Ac | 337.3195 | 2.6236 |
| hexanoic acid, 4-hexadecyl ester | 341.3394 | 100 |
| 5,8,11,14-Eicosatetraenoic acid | 348.2986 | 2.2168 |
| Bahiensol | 349.2859 | 1.2899 |
| Fawcettiine;Ac | 350.2261 | 0.0201 |
| 8,11,14-Eicosatrienoic acid | 350.3042 | 0.6317 |
| 4-Methyl Sucrose | 357.1462 | 0.0608 |
| Emericolin B;Emericolin C | 357.3143 | 7.2486 |
| Anilide Octadecanoic acid | 360.3309 | 3.3824 |
| 13-Epiyosgadensonol | 363.2899 | 2.6753 |
| Eicosanedioic acid;Di-Me ester | 371.3241 | 50.1742 |
| 2,4,16-Eicosatrienoic acid | 374.347 | 3.3999 |
| cholestenone/cholecalciferol | 385.3494 | 5.6362 |
| Barrenazine A | 387.3419 | 3.3365 |
| N3,N3-Di-Methyl, N20-Acetyl | 389.3513 | 3.7561 |
| octyl phthalate | 391.2825 | 73.7191 |
| 24-Nor-18a-olean-12-ene | 397.3835 | 17.8697 |
| campesterol | 401.3729 | 25.1522 |
| cholesteryl chloride | 405.331 | 1.9594 |
| 12,21-Baccharadiene | 411.3955 | 33.2776 |
| 2-Amino-2,3-dideoxy-ribo-hexose | 412.1838 | 0.0968 |
| fucosterol/sitosterone | 413.3858 | 7.0665 |
| Buxidienine F | 417.3482 | 2.2051 |
| maesaquinone | 419.3196 | 26.7388 |
| amyrenone/lupenone | 425.3768 | 28.4833 |
| 10,11-Epoxysqualene | 427.3929 | 11.1286 |
| 5,6-Epoxystigmastan-3-ol | 431.3823 | 1.7737 |
| 26-Amino-3,16-dihydroxychole | 434.3536 | 1.2177 |
| 12-Oleanene-3,22-diol | 443.396 | 5.7247 |
| 6,22-Hopanediol | 445.3978 | 2.3505 |
| Farnesyl farnesylcarboxylate | 455.3922 | 2.9039 |
| soyasapogenol B | 458.3812 | 3.3835 |
| panaxadiol/protopanaxadiol | 461.3998 | 1.5575 |
| Balansenate I | 479.4877 | 7.8653 |
| Ergosta-7,22-diene-3,5,6-triene | 487.4225 | 1.8526 |
| Didodecyl phthalate | 503.4184 | 3.6387 |
| Fasciculin A, Tetra-Me ether | 505.4983 | 2.9293 |
| 4-Methylhomoindanomycin | 522.3668 | 0.0693 |
| Balansenate II | 535.5408 | 6.9714 |
| Protosappanin E2 | 587.5624 | 1.3202 |
| pelargonin/cyanidin rutinoside | 596.1765 | 0.0969 |
| Ergost-5-en-3-ol;Hexadecanoyl | 639.616 | 1.0839 |
| Ergosta-7,22-diene-3,5,6-triene | 669.5919 | 0.3768 |
| Ergost-5-en-3-ol;13E-Docosenol | 721.6909 | 0.3148 |

TABLE 5
Summary of the identified compounds in Extract 5 as
determined by DART TOF-MS.

| | | Relative |
|---|---|---|
| | Measured | Abundance |
| Compound Name | Mass | (%) |
| aminobutyric acid | 104.0723 | 78.9324 |
| catechol/resorcinol | 111.0485 | 1.3311 |
| uracil | 113.0259 | 0.7342 |
| butyl isothiocyanate | 116.0519 | 0.5256 |
| levulinic acid | 117.0499 | 0.6583 |
| L-threonine | 120.063 | 0.552 |
| pyrogallol/phlorglucinol | 127.0413 | 1.8299 |
| amyl acetate/caproic acid methyl | 131.1138 | 0.7708 |
| ester | | |
| aminolevulinic acid | 132.0677 | 16.852 |
| glutaric acid | 133.0542 | 0.7792 |
| 4-hydroxybenzoic acid | 139.0412 | 2.4747 |
| kojic acid/muconic acid | 143.0314 | 0.4038 |
| 3-hydroxy-2,3-dihydromaltol | 145.0503 | 2.7203 |
| galactal | 147.0678 | 3.7225 |
| L-glutamine | 147.0678 | 3.7225 |
| 4-hydroxyisoleucine | 148.0951 | 4.9638 |
| nornicotine | 149.1164 | 35.1684 |
| carvacrol/thymol/cymenol | 151.1068 | 2.3987 |
| dihydroxyacetophenone | 153.0585 | 0.8032 |
| 2,3-dihydroxybenzoic acid | 155.0356 | 1.5457 |
| methyl-2-octynoate | 155.103 | 0.4354 |
| methylcoumarin | 161.0508 | 0.5616 |
| L-2-aminoadipic acid | 162.0854 | 2.3644 |
| allicin | 163.0203 | 0.168 |
| shikimic acid | 175.0603 | 9.849 |
| DL-a aminopimelic acid | 176.0921 | 10.612 |
| cinnamyl acetate | 177.1005 | 0.6079 |
| alliin | 178.0514 | 0.2815 |
| glucose | 180.0695 | 0.2911 |
| adrenochrome | 180.0695 | 0.2911 |
| L-adrenaline | 184.0918 | 2.8756 |
| angelicin | 187.0439 | 0.1277 |
| n-acetyl-L-glutamine | 189.0904 | 0.4972 |
| 4-phenylbutylisothiocyanate | 192.0945 | 2.7901 |
| quinic acid | 193.0694 | 13.8757 |
| a-phenylindol | 194.101 | 4.1657 |
| L-dopa | 198.0737 | 1.6439 |
| citronellyl acetate | 199.1664 | 11.4321 |
| harmalol | 201.1 | 1.7504 |
| chitin | 204.083 | 1.1107 |
| 4-methyl-7-ethoxycoumarin | 205.0837 | 3.2062 |
| eugenol acetate | 207.0981 | 0.7442 |
| hydrastinine | 208.1037 | 0.5382 |
| 3-methoxy-1-tyrosine | 212.0911 | 6.8283 |
| benzyl benzoate | 213.0949 | 1.012 |
| 6-furfurylaminopurine | 216.0855 | 4.0199 |
| vitamin B5 | 220.1166 | 59.9204 |
| hydrocotarnine | 222.1165 | 0.2392 |
| 6-benzylaminopurine | 226.1002 | 0.5024 |
| kavain | 231.106 | 9.3793 |
| dihydrokavain | 233.1084 | 1.079 |
| 6-aminochrysene | 244.1054 | 0.9326 |
| 4-methylumbelliferyl butyrat | 247.107 | 2.4814 |
| methoxyflavanone | 255.1115 | 0.7065 |
| (+/−)-n-acetyl homotryptophan | 260.1122 | 1.7848 |
| lotaustralin | 262.1283 | 3.5189 |
| abscisic acid | 265.1377 | 15.3909 |
| magnolol | 267.1335 | 100 |
| vestitol | 273.1211 | 0.537 |
| piperine | 286.1357 | 1.0435 |
| salicin | 287.1177 | 0.5281 |
| rutaecarpine | 288.1165 | 0.351 |
| galanthamine | 288.1638 | 0.2681 |
| 6-dehydrogingerdione | 291.1528 | 6.3476 |
| pukateine | 296.1308 | 2.2519 |
| salidroside | 301.1378 | 1.2711 |
| enterodiol | 303.1556 | 4.2594 |
| nordihydroguaiaretic acid | 303.1556 | 4.2594 |
| scopolamine | 304.1558 | 1.1864 |
| zearaleone | 319.1472 | 0.7563 |
| bulbocapnine | 326.1467 | 0.9916 |
| seneciphyllin | 334.1722 | 2.2525 |
| bavachinin A/bergamotin | 339.1695 | 1.4875 |
| papaverine/tetrahydroberberine | 340.1586 | 0.4631 |
| corydine/corypalmine/luteanin | 342.171 | 0.7816 |
| S-petasine | 349.1752 | 1.0213 |
| serpentine | 350.1541 | 1.8596 |
| retrorsine | 352.1741 | 1.4427 |
| aldosterone/cortisone | 361.1919 | 0.7856 |
| senkirkin | 366.1888 | 0.1648 |
| corycavine | 368.1571 | 0.9394 |
| uncarine/mitraphylline | 369.1902 | 9.3463 |
| corydaline | 370.1925 | 1.7725 |
| fraxin | 371.092 | 0.626 |
| tetrahydrocurcumin | 373.1689 | 1.0974 |
| cornin/geniposide | 389.1469 | 0.8501 |
| loganin | 391.1704 | 1.0117 |
| colchicine | 400.1804 | 0.4774 |
| valtrate | 409.194 | 0.5328 |
| linustatin | 410.1701 | 2.5711 |
| schisandrol B | 417.1847 | 1.2773 |
| rosarin/rosavin | 429.1802 | 0.2241 |
| biochanin A glucoside | 447.1277 | 1.9149 |
| madecassic acid/pygenic acid | 505.3624 | 0.6107 |

TABLE 6
Compounds identified in Extract 6 by DART TOF-MS.

| | | Relative |
|---|---|---|
| | Measured | Abundance |
| Compound Name | Mass | (%) |
| 3-Aminodihydro-2(3H)-furanone | 102.0505 | 0.0625 |
| Farmiserina | 103.0439 | 0.2314 |
| 1,4-benzoquinone | 109.0285 | 16.0092 |
| 1,2-Benzenediol | 111.0455 | 1.6796 |
| 2-Hydroxypropanoic acid | 113.0246 | 2.4631 |
| 5-azauracil | 114.0387 | 0.4221 |
| 4-methylene-heptane | 114.1469 | 0.0169 |
| 5-Hydroxymethyl-2(5H)-furanone | 115.0431 | 3.586 |
| octane | 115.157 | 0.0331 |
| butyl isothiocyanate | 116.0484 | 0.3459 |
| indole | 118.071 | 0.1563 |
| Succinic acid | 119.037 | 10.1939 |
| L-threonine | 120.0604 | 4.724 |
| Benzoic acid | 123.0516 | 1.3274 |
| niacin | 124.0441 | 0.7678 |
| 4-methyl-5-vinylthiazole | 126.0375 | 5.6176 |
| pyrogallol/phlorglucinol | 127.0389 | 100 |
| 2-ethyl-4-methylthiazole | 128.0448 | 4.6732 |
| 1,3-Dicyanobenzene | 129.0521 | 1.705 |
| aminolevulinic acid | 132.0603 | 24.168 |
| cinnamaldehyde | 133.0653 | 5.878 |
| 2-Cyano-6-methylphenol | 134.0687 | 1.1551 |
| malic acid | 135.0323 | 2.2009 |
| Adenine | 136.0618 | 0.3388 |
| anisaldehyde | 137.0623 | 0.864 |
| 4-Aminobenzoic acid | 138.0638 | 0.226 |
| 4-hydroxybenzoic acid | 139.0422 | 7.849 |
| 3-Acetyl-4- | 140.0711 | 0.9249 |
| (hydroxymethyl)pyrrole | | |
| furfuryl acetate | 141.061 | 0.3173 |
| kojic acid | 143.0368 | 3.3061 |
| 1,4-Dihydroxy-2-cyclopentene | 144.0598 | 5.1267 |
| 1-methyl-5-Fluoro-2,4(1H,3H)- | 145.0497 | 91.2625 |
| pyrimidinedione | | |
| 3-Phenyloxiranecarboxylic ac | 146.0549 | 6.5369 |
| coumarin | 147.0455 | 17.9508 |
| Benzazide | 148.0547 | 1.5883 |
| anethole/cuminaldehyde | 149.061 | 7.799 |
| chitosan | 150.0716 | 1.900 |
| 2-Phenylethyl formate | 151.0735 | 0.7705 |
| guanine | 152.0628 | 1.6054 |
| dihydroxyacetophenone | 153.0638 | 0.5072 |
| Scopine-3-Ketone | 154.0894 | 0.4515 |
| Fluoride Methoxybenzoic acid | 155.0499 | 0.4808 |
| 5-(Methoxymethyl)-2-furan- | 157.0512 | 3.6329 |
| carboxylic acid | | |
| allantoin | 159.0569 | 0.5291 |
| betonicine/acetyl valine | 160.0971 | 0.367 |
| 2,3-Oxiranedicarboxylic acid | 161.0533 | 1.2962 |
| L-2-aminoadipic acid | 162.0747 | 1.5886 |
| glyogen | 163.0629 | 8.9555 |
| phenylethyl isothiocyanate | 164.0551 | 1.7537 |
| Diamide 1,3-Benzenedicarboxy | 165.0671 | 1.3622 |
| 4-(Ethylamino)benzoic acid | 166.0833 | 0.5008 |
| phenyllactic acid | 167.0789 | 0.5178 |
| 4-Amino-3-methoxybenzoic acid | 168.0759 | 0.6364 |
| norharman | 169.0824 | 2.2396 |
| vitamin B6 | 170.0839 | 0.9101 |
| 2-Acetyl-3,5-dihydroxy-2-cyclo- | 171.0698 | 0.8481 |
| hexen-1-one | | |
| Me ether Zymonic acid | 173.0503 | 1.4926 |
| 1-(2-hydroxyehtyl)-5-fluoro- | 175.0596 | 5.9 |
| 2,4(1H,3H)-pyrimidinedione | | |
| 6-Hydroxy-7-methoxyisoquinol | 176.072 | 0.8632 |
| 4-methylumbelliferone | 177.0585 | 1.6307 |
| 3-Phenyloxiranecarboxylic acid | 178.0839 | 0.729 |
| 1-(5-Hydroxy-2-methylphenyl)- | 179.0711 | 1.3547 |
| 1,2-propanedione | | |
| 1-Amino-1-deoxyfructose | 180.0875 | 5.075 |
| 2-Deoxy-arabino-hexonic acid | 181.0796 | 2.2548 |
| Erbstatin;1′,2′-Dihydro | 182.0873 | 1.1833 |
| 1,3,11-Tridecatriene-5,7,9-triyne | 183.0781 | 1.4616 |
| epinephrine | 184.0969 | 0.4861 |
| 4,5-Bis(hydroxymethyl)-2- | 185.0845 | 0.676 |
| methyl-1,3-benzenediol | | |
| Erinapyrone C | 187.0628 | 3.2042 |
| N-(3- | 188.0859 | 0.9001 |
| Hydroxybutanoyl)homoserine | | |
| Enteromycin | 189.0587 | 2.2621 |
| Citric acid | 190.0806 | 0.8893 |
| 1,3-di-tert-butylbenzene | 191.1808 | 4.4682 |
| 5-hydroxyindolyl-3-acetic acid | 192.0741 | 1.8246 |
| 3-(bromomethyl)-heptane | 193.0688 | 20.0807 |
| a-phenylindol | 194.0770 | 7.262 |
| ferulic acid | 195.072 | 1.7324 |
| DL-a-methyl-m-tyrosine | 196.1016 | 0.8701 |
| 2′,4′-Dihydroxy-6′-methoxy-3 | 197.0864 | 0.7958 |
| 2-Hydroxy-7-methyl-9H- | 198.0988 | 7.8663 |
| carbazole | | |
| Harmol | 199.0955 | 1.3841 |
| 2-(2,4-hexadiynylidene)-1,6- | 201.0969 | 1.0103 |
| dioxaspiro[4.4]non-3-ene | | |
| 3,6-Dideoxy-erythro-hexo- | 202.1146 | 0.566 |
| pyranos-4-ulose | | |
| 1,3-benzenedicarboxylic acid | 202.9625 | 0.0055 |
| dichloride | | |
| 1(10),8,11-Eremophilatriene | 203.1806 | 2.0222 |
| 8-Epialexaflorine | 204.084 | 1.1479 |
| 1-(2-hydroxy-2-methoxyethyl)-5- | 205.0671 | 7.6852 |
| fluoro-2,4(1H,3H)- | | |
| pyrimidinedione | | |
| Edulitine | 206.0795 | 1.5557 |
| citropten | 207.0727 | 7.3426 |
| hydrastinine | 208.0936 | 1.9122 |
| Allaric acid Diamide | 209.0854 | 1.7092 |
| Thiolactomycin | 211.072 | 2.3437 |
| Enicoflavine | 212.0948 | 1.7461 |
| 2-(2,4-Hexadiynylidene)-5- | 213.0968 | 1.7276 |
| (propionylmethylidene)-2,5- | | |
| dihydrofuran | | |
| a-Allokainic acid | 214.106 | 0.6419 |
| Demethylaaptamine | 215.0908 | 0.6027 |
| 6-furfurylaminopurine | 216.0975 | 0.5169 |
| 5,6-O-Isopropylidene-L-threonine | 217.0647 | 3.3265 |
| captopril(usp) | 218.0899 | 1.3357 |
| 5E-Zeyherin | 219.0673 | 9.1143 |
| 1-benzyl-5-fluoro-2,4(1H,3H)- | 221.0822 | 1.1828 |
| pyrimidinedione | | |
| fraxidin | 223.0626 | 8.1597 |
| 2-Methylfervenulone | 224.0803 | 1.7948 |
| 3,5-dimethoxy-4-hydroxy | 225.0796 | 4.3292 |
| cinnamic acid | | |
| Epidestomic acid | 226.099 | 1.0099 |
| Aspyrone | 227.0975 | 1.1089 |
| 2′-Deoxycytidine | 228.0972 | 0.5304 |
| rezazurin | 229.0818 | 2.2203 |
| Ergothioneine | 230.1033 | 1.1232 |
| 1,4:3,6-Dianhydromannitol, 2,5- | 231.0954 | 1.3057 |
| Di-Ac | | |
| N-Benzoyl Baikiain | 232.1071 | 0.6004 |
| Fadyenolide | 233.0763 | 1.8784 |
| 8-acetyl-6-hydroxy-7- | 235.0616 | 18.4465 |
| methoxycoumarin | | |
| 2-Amino-2-deoxygalacturonic | 236.0781 | 3.8751 |
| acid | | |
| 7-(2-Hydroxyethoxy)-6-methoxy- | 237.0831 | 4.3045 |
| 2H-1-benzopyran-2-one | | |
| Eritadenine;Deoxyeritadenine | 238.092 | 1.3491 |
| 3-Deoxy-manno-oct-2-ulosonic | 239.0824 | 1.5672 |
| acid | | |
| 6N-Benzoyl Adenine | 240.0966 | 0.7118 |
| Scytolide | 241.0755 | 4.2887 |
| 4-Nitrophenylhydrazone | 242.0942 | 1.1255 |
| Benzaladehyde | | |
| Fructose 2-Chloroethyl glycoside | 243.0636 | 9.566 |
| 6-Amino-3-ribofuranosyl-4(3H)- | 244.0859 | 1.5197 |
| pyrimidinone | | |
| biotin | 245.0871 | 1.767 |
| 2,6-Dideoxy-3-C-methyl- | 247.1159 | 3.6632 |
| arabinoside | | |
| 4-Amino-4,6-dideoxy-3-C- | 248.1478 | 1.3621 |
| methylmannose Me glycoside | | |
| 2,5-Anhydroglucitol, 1,3,4-Tri- | 249.1397 | 3.1727 |
| Me | | |
| 2-Acetamido-2-deoxyglucose 3,4- | 250.1333 | 0.9623 |
| Di-Me | | |
| N,N′-Dimethyl-N,N′-dinitroso- | 251.0717 | 1.0402 |
| 1,4-benzenedicarboxamide | | |
| Bis(2-hydroxyethyl) ester 1,4- | 255.0869 | 2.8572 |
| Benzenedicarboxylic acid | | |
| 2-[[(3- | 256.1013 | 1.4758 |
| Methylphenyl)amino]carbonyl]- | | |
| benzoic acid | | |
| Norbaeocystine | 257.0782 | 2.1881 |
| Lamiophlomiol C | 259.0885 | 5.1127 |
| Pyrazofurin | 260.0914 | 1.9196 |
| Lambertellol B | 261.0836 | 5.1355 |
| alahopcin | 262.1067 | 1.082 |
| 1,2-Diphenoxybenzene | 263.1149 | 1.9948 |
| 2-Acetamido-2-deoxyglucose-3- | 264.1142 | 0.7351 |
| Ac | | |
| abscisic acid | 265.1215 | 31.212 |
| Vitamin B1 | 266.126 | 0.815 |
| 7-Methoxy-2-methylisoflavone | 267.098 | 3.1994 |
| Zefbetaine | 268.1007 | 2.5332 |
| 13-Dehydromaturin | 269.0851 | 2.74 |
| Baeocystine | 271.0824 | 66.7588 |
| 4-Hydroxydianthramide B methyl | 272.093 | 10.8659 |
| ester | | |
| vestitol | 273.0828 | 1.986 |
| Benzaldehyde tosylhydrazone | 275.0922 | 6.7409 |
| Osmaronin epoxide | 276.1178 | 3.1963 |
| Ellipticine | 277.1043 | 6.5243 |
| Spirostaphylotrichin H | 278.1078 | 2.1622 |
| Eleostearic acid | 279.2387 | 2.5251 |
| 4-(6-Amino-9H-purin-9-yl)-1- | 280.095 | 0.9684 |
| (hydroxymethyl)-6- | | |
| oxabicyclo[3.1.0]hexane-2,3-diol | | |
| a-Isobromocuparene | 281.1162 | 2.383 |
| 3′,4′-dimethoxyflavone | 283.1044 | 8.6073 |
| Anaxagoreine | 284.1188 | 2.1978 |
| helicin | 285.107 | 4.8519 |
| 2-Amino-2-deoxyglucose Di-Et | 286.1208 | 1.6103 |
| dithioacetal | | |
| homobutein | 287.0893 | 2.3235 |
| Taraktophyllin | 288.1141 | 2.7116 |
| Hydroxyanigorufone | 289.0939 | 52.3519 |
| 1,2,3,4-Tetra-O-acetyl-DL- | 291.0992 | 9.5663 |
| threitol | | |
| Epoxysarmentosin | 292.1064 | 2.7123 |
| 5′-Epialtenuene | 293.1056 | 3.033 |
| Muramic acid;1′-Epimer, N-Ac | 294.1196 | 1.1654 |
| Conocandin | 295.2289 | 18.1237 |
| 2,5-Epoxy-6,10,14-trimethyl- | 297.2447 | 2.958 |
| 9,13-pentadecadiene-2,6-diol | | |
| octylgallate | 299.1421 | 3.5378 |
| Erythrartine | 300.1249 | 1.305 |
| 4,9-Anhydro-6-epitetrodotoxin | 302.1044 | 4.078 |
| Galacturonic acid | 303.0663 | 11.6466 |
| 1,5-Anhydrofructose, Tri-Ac, | 304.1041 | 2.661 |
| oxime | | |
| 2-Acetamido-2-deoxyglucose D | 306.1225 | 3.0439 |
| Galactose | 307.1063 | 30.7207 |
| bisdemethoxycurcumin | 309.1091 | 4.7034 |
| n-acetylneuraminic acid | 310.1214 | 2.5235 |
| 2,6,10-Farnesatrien-1-oic acid | 311.2271 | 1.6198 |
| 2-Amino-2-deoxygalactose | 312.1539 | 1.2905 |
| 2-Amino-2-deoxyglucose N- | 314.126 | 1.7291 |
| Benzyloxycarbonyl | | |
| 1,4-Benzenediol;1-Ac, 4-O-b-D- | 315.1143 | 4.7404 |
| galactopyranoside | | |
| Maremycin D2 | 316.1299 | 1.4613 |
| 1,5-Anhydroglucitol, 2-O-(3,4,5- | 317.0802 | 8.834 |
| Trihydroxybenzoyl) | | |
| 2-Amino-2-deoxyxylose Tetra-Ac | 318.1096 | 4.1651 |
| 1,4-Benzenediol;Dibenzoyl | 319.1048 | 25.5834 |
| 4-Epitetrodotoxin | 320.1167 | 5.2651 |
| Erythrinin A | 321.1176 | 4.763 |
| 13(11→12)-Abeo-7,11,15- | 323.1586 | 3.1284 |
| trihydroxy-1,3-eudesmadiene- | | |
| 8,12-dione | | |
| 11-Methyl ether Atalaphyllidine | 324.1329 | 1.1515 |
| 5-(Methoxycarbonyl)tubercidin | 325.1196 | 17.0488 |
| rutinose | 326.1307 | 2.3737 |
| Plakortide Q | 327.2606 | 4.3088 |
| 15,16-Epoxy-12-oxo-7,13(16),14- | 329.1851 | 2.8682 |
| labdatrien-19,6-olide | | |
| Lycorine;Poetaminine | 330.1366 | 0.9148 |
| 3,4-Epoxypalisadin A | 331.1204 | 5.9134 |
| 2′-Deoxycytidine, 4N-Benzoyl | 332.1231 | 1.739 |
| 6-Deoxyglucose, 1,2,3,4-Tetra-Ac | 333.1195 | 6.6032 |
| 2-Amino-2-deoxyglucose Et | 334.1491 | 1.9101 |
| glycoside, 3,4,6-tri-Ac | | |
| Fasicularine | 335.2445 | 4.1006 |
| 3-Acetyl-o-methyl-o-(4- | 336.1927 | 1.8058 |
| oxodecanoyl)histidine | | |
| Crotafuran C | 337.1173 | 9.2383 |
| Emethacin A | 339.1191 | 2.9939 |
| linocinamarin | 341.1312 | 1.3608 |
| 2-Acetamido-2-deoxyglucose 4- | 343.1218 | 4.2316 |
| Nitrophenyl glycoside | | |
| Bauhinin | 344.1356 | 1.0041 |
| Macrosphelide I | 345.1564 | 3.2163 |
| Fareanine | 348.1146 | 1.8327 |
| S-petasine | 349.1309 | 4.5309 |
| Erucifoline | 350.1696 | 1.639 |
| vinpocetine | 351.1993 | 5.5239 |
| 10-gingerdiol | 353.2749 | 3.3494 |
| rubrocyanin | 354.2153 | 2.0021 |
| 11,12-Didehydroplakortide Q | 355.2859 | 4.777 |
| 8-Epigalbulin | 357.1992 | 3.0812 |
| Erigeside C | 361.1196 | 4.3067 |
| 8,8,9-trimethoxy-5- | 362.1401 | 1.5356 |
| methylbenz[cd]isoindolo[2,1- | | |
| a]indol-1(8H)-one | | |
| hydrocortisone | 363.22 | 3.1094 |
| 1-Epidioncophylline B | 364.1977 | 1.2673 |
| 14,15-Epoxy-3-oxovincadifformine | 367.1662 | 3.9631 |
| Edulane | 369.1795 | 4.6287 |
| Intermedine | 370.23 | 1.84 |
| Fraxin | 371.2818 | 0.3353 |
| 16,17-Dihydro-17- | 373.2205 | 0.9053 |
| demethoxyisorhyncophylline N- | | |
| oxide | | |
| Bengamide Y | 375.2065 | 1.8875 |
| simmondsin | 376.1601 | 0.5464 |
| 7-Epidionocophylline A | 378.2071 | 1.2675 |
| 5-Hydroxychelirubine | 379.1098 | 3.7018 |
| Bocconoline | 380.1464 | 1.8658 |
| Badrakemone | 381.2068 | 2.2035 |
| 7-Angelylheliotridine trache | 382.2189 | 1.3195 |
| Ergosta-7,22-diene | 383.3676 | 6.0526 |
| piscodone | 385.1321 | 2.7851 |
| Segoline C | 386.1589 | 1.1823 |
| Septentriosine | 388.2176 | 1.2431 |
| Ambiguine C isonitrile | 389.2579 | 1.9833 |
| 2-Epi-2-O-ethylcephalo- | 390.2195 | 0.7288 |
| fortuneine | | |
| 24-Nor-18a-olean-12-ene | 397.3851 | 28.8986 |
| 11(15→1)-Abeo-2,20-epoxy-11- | 401.2181 | 3.1032 |
| taxene-4,5,7,9,10,13,15-heptol | | |
| dehydrocholic acid | 403.2441 | 2.559 |
| 19-Malonylkingidiol | 405.2346 | 4.6876 |
| 2,6,10,15,19,23-Hexamethyl- | 409.388 | 17.4218 |
| 2,6,10,12,14,18,22- | | |
| tetracosaheptaene | | |
| 24-Epicyclonervilasterol | 411.3659 | 24.5123 |
| fucosterol/sitosterone/spinasterol | 413.3815 | 5.7809 |
| Edulone A | 415.1441 | 5.0005 |
| schisandrol B | 417.2669 | 3.1929 |
| amyrenone/lupenone | 425.3747 | 5.5169 |
| Nb-Octadecanoyltryptamine | 427.3776 | 4.1564 |
| cholesteryl acetate | 429.3806 | 6.6869 |
| 5,6-Epoxystigmastan-3-ol | 431.3913 | 5.21 |
| 6-Epiacetylscandoside | 433.1399 | 2.0422 |
| Delelatine | 436.2609 | 0.4492 |
| Ergosta-4,6,8(14),22-tetraen-3- | 437.3597 | 2.1846 |
| ylurea | | |
| 29(20→19)-Abeo-3-hydroxy-20- | 439.3581 | 100 |
| lupanone | | |
| 12-Oleanene-3,22-diol | 443.3818 | 2.9873 |
| 5,6-Epoxystigmast-8(14)-ene-3,7- | 445.3705 | 2.6075 |
| diol | | |
| 1,3,5-Trihydroxyergost-24(28)- | 447.3475 | 1.169 |
| en-6-one | | |
| Ellagic acid;Ducheside A | 449.0658 | 0.0136 |
| 6-Deoxodolichosterone | 449.3533 | 1.0083 |
| condelphine | 450.2946 | 0.6197 |
| 4,5-Epoxy-2,8,13-trihydroxy- | | |
| 1(10),7(11)-germacradien-12,6- | 451.1615 | 3.9632 |
| olide | | |
| Papuamine | 453.3529 | 1.8477 |
| 14b,26-Epoxy-3,21-serratan- | 455.362 | 3.7047 |
| edione | | |
| 1,11-Epidioxy-12-ursen-3-ol | 457.3705 | 29.6716 |
| soyasapogenol B | 458.3761 | 10.1773 |
| 11(12→13)-Abeo-3,11- | 459.3901 | 3.0868 |
| dihydroxy-12-oleananal | | |
| Ajugoside | 469.1655 | 0.6337 |
| keto boswellic acid/glycyrrhetinic | 471.3534 | 2.0057 |
| acid | | |
| 19(10→9)-Abeo-4,5-epoxy-3,4- | 479.4116 | 1.5829 |
| secotirucallane-3,24,25-triol | | |
| Broussonetine X | 488.2929 | 0.5203 |
| Batzelladine C | 489.3998 | 1.2222 |
| Emindole PA | 490.3754 | 0.6161 |
| 3,3′,4′,5,7-Pentahydroxyflavone | 491.1538 | 0.0507 |
| 21,22-Epoxy-3,20-taraxastane | 501.403 | 1.1234 |
| ganoderiol A | 503.4046 | 1.2616 |
| Deacetylisoipecoside | 524.2078 | 0.0234 |
| Nb-Pentacosanoyltryptamine | 525.4806 | 1.438 |
| Alvaradoin B | 527.1838 | 0.084 |
| betulin diacetate | 527.4156 | 0.657 |
| Buxhejramine | 529.4379 | 0.9658 |
| Tetracosyl (E)-ferulate | 531.4433 | 1.1162 |
| Justisolin;O-b-D-Glucopyranoside | 533.1727 | 0.0368 |
| Ergost-22-en-3-ol | 533.413 | 0.7676 |
| β-D-Glucopyranosyl ester, | 537.1883 | 0.0373 |
| Deacetylhookerioside | | |
| b-Carotene | 537.452 | 1.3429 |
| kutkoside | 538.177 | 0.0504 |
| 29-(2,3,4,5-Tetrahydroxypentyl)- | 543.4351 | 1.0979 |
| 6,11-hopadiene | | |
| Gummadiol | 549.1654 | 0.0663 |
| 5-Deoxyarabinitol, Tetrabenzoyl | 553.1873 | 0.0711 |
| tricaprin | 555.4647 | 0.686 |
| 29-(1,2,3,4,5- | 563.4706 | 0.7171 |
| Pentahydroxypentyl)hopane | | |
| alloxanthin | 565.4051 | 0.9525 |
| Heptacosyl (E)-ferulate | 573.4957 | 2.0733 |
| Epiactephilol A | 581.2176 | 0.0983 |
| Myxovirescin G2 | 582.4397 | 0.4286 |
| amarogentin | 587.177 | 0.0107 |
| Oscillatoxin B2 | 591.3161 | 0.0325 |
| Reticulatain 2 | 593.5061 | 0.9837 |
| 10,18-Epoxy-1(19),7,11,13- | 599.51 | 7.5759 |
| xenicatetraene-6,17-diol | | |
| Dihydroergocristine | 612.3197 | 0.0023 |
| 3-O-B-D-Glucuronopyranoside | 625.1416 | 0.0222 |
| 4,7′-Epoxy-3,8′-bilign-7-ene | 625.2633 | 0.01 |
| Ac, octacosyl ester | 629.5082 | 0.8038 |
| trilaurin | 639.5516 | 1.0679 |
| Calamistrin B | 651.5284 | 0.6863 |
| Diosgenin palmitate | 653.5434 | 0.4249 |
| Ergocerebrin | 666.6494 | 0.5086 |
| Ergost-5-en-3-ol | 667.6436 | 0.2987 |
| Giganteumgenin D | 675.4448 | 0.0587 |
| 3-O-Hexadecanoyl | 695.598 | 0.13 |
| Bisanhydrobacterioruberin | 705.5624 | 0.2657 |
| Belamcandone A | 709.4999 | 0.5204 |
| 3′,4′- | 739.5722 | 0.3348 |
| Epoxymonoanhydrobacterioruberin | | |
| Bacterioruberin | 741.5863 | 0.7274 |
| Manzamenone B | 743.5831 | 0.7356 |
| Yendolipin | 763.6087 | 0.4793 |

B. Biostatic Activity

The biostatic (inhibition of growth) activity of the Extract 5 against C. albicans was determined by generating growth curves, while the biostatic activity of Extract 6 was examined against C. albicans, S. aureus and E. coli. For Extract 5, an IC₅₀ value for inhibition of growth was reached at 676 μg mL⁻¹ (Table 6). For Extract 6, the dose-dependent inhibition of C. albicans growth was achieved at an IC₅₀ value of 75.2 μg mL⁻¹. The dose-dependent inhibition of E. coli growth was achieved by Extract 6 at an IC₅₀ value of 305.7 μg mL⁻¹. The IC₅₀ value of 288 μg mL⁻¹ was obtained for dose-dependent inhibition of S. aureus growth with Extract 6. This data is summarized in Table 7.

TABLE 7
Biostatic activities of Extract 5 and Extract 6 against C. albicans, E. coli,
and S. aureus.

| | ATCC 96133 | | | | | | | | |
|---|---|---|---|---|---|---|---|---|---|
| | C. albicans | | | ATCC 53499 E. coli | | | ATCC 700787 S. aureus | | |
| | IC₅₀ | | | IC₅₀ | | | IC₅₀ | | |
| | (μg mL⁻¹) | R² | N | (μg mL⁻¹) | R² | N | (μg mL⁻¹) | R² | N |
| Extract 5 | 678 | 0.96 | 14 | NA | NA | NA | NA | NA | NA |
| Extract 6 | 75 | 0.96 | 21 | 306 | 0.86 | 21 | 288 | 0.89 | 24 |

C. Anti-Adhesion Activity

The IC₅₀ values for adhesion inhibition of C. albicans for Extract 2, Extract 3, Extract 4, Extract 5, and Extract 6 were 95.9 μg mL⁻¹, 799.7 μg mL⁻¹, and 14.6 μg mL⁻¹, 168 μg mL⁻¹, and 92.3 μg mL⁻¹, respectively (Table 8). The IC₅₀ values for adhesion inhibition of E. coli for Extract 2, Extract 3, Extract 4, and Extract 6 were 31.5 μg mL⁻¹, 13.1 μg mL⁻¹, and 42.8 μg mL⁻¹, and 1.5 μg mL⁻¹, respectively. Data is summarized in Table 8.

TABLE 8
Anti-adhesion activities of Extract 2, Extract 3, Extract 4, Extract 5
and 6 against C. albicans, and E. coli

| | ATCC 96133 C. albicans | | | ATCC 53499 E. coli | | |
|---|---|---|---|---|---|---|
| HS | IC₅₀ | | | IC₅₀ | | |
| Number | (μg mL⁻¹) | R² | N | (μg mL⁻¹) | R² | N |
| Extract 2 | 95.9 | 0.845 | 31 | 31.5 | 0.943 | 38 |
| Extract 3 | 799.7 | 0.816 | 32 | 13.1 | 0.973 | 35 |
| Extract 4 | 14.6 | 0.916 | 31 | 42.8 | 0.908 | 47 |
| Extract 5 | 168.0 | 0.970 | 16 | ND | ND | ND |
| Extract 6 | 92.3 | 0.97 | 21 | 1.47 | 0.78 | 12 |

(ND = not determined).

D. Direct Binding of Anti-adhesion Chemistries

The DART-MS of C. albicans cells that were incubated in the cranberry extract and washed free of unbound chemistries was used to identify the active compounds in the extract (B. Roschek Jr., R. C. Fink, M. D. McMichael, D. Li and R. S. Alberte, 2009. Elderberry flavonoids bind to and prevent H1N1 Infection in vitro. Phytochemistry. In Press). The bound compounds present in the extract are inhibitors of C. albicans adhesion and function by binding to C. albicans blocking its ability to adhere to cells.

E. Post-Binding Assay

In the post-binding assays conducted after the anti-adhesion bioactives were allowed to bind to the pathogen and non-bound compounds were removed, showed that the identified bioactives block the ability of C. albicans, E. coli and S. aureus from attaching/adhering as a result of their presence on the surface of the pathogen. This re-confirms the anti-adhesion mode-of-action of the cranberry extracts and the key bioactives. The data is summarized in Tables 9-11.

In Table 9, adhesion and post-binding adhesion are summarized for C. albicans challenged with cranberry Extracts 5 and 6. When C. albicans has bound bioactives from cranberry Extract 6 or Extract 5, adhesion is inhibited. At 1000 μg ml⁻¹ of Extract 6, in excess of the IC₁₀₀ value for anti-adhesion, the percent inhibition for adhesion after bioactives are bound (post-binding assay) is essentially identical to that in the initial adhesion assay. When C. albicans is incubated at 100 μg mL⁻¹ of Extract 6, a 20% reduction in adhesion was observed, whereas when only the bound chemistries are present there is a 60% inhibition of adhesion. When C. albicans was incubated in 1000 μg ml⁻¹ Extract 5, the percent inhibition of adhesion after bioactives are bound (post-binding assay) is approximately 1.5 times that observed in the adhesion assay. When C. albicans was incubated in 100 μg mL⁻¹ of Extract 5, a similar increase in the inhibition of adhesion due to the binding of extract bioactives was observed.

When bioactives from Extract 6 are bound to E. coli, adhesion is inhibited (Table 10). Incubation of E. coli with 1000 μg ml⁻¹ of Extract 6, the percent inhibition for adhesion after bioactives are bound (post-binding assay) is essentially identical to that found in the adhesion assay (Table 10). When the E. coli is incubated at 100 μg ml⁻¹ of Extract 6 and only bound chemistries are present, the inhibition of attachment is greater than that observed in the presence of the whole Extract 6. This is most likely due to the presence of compounds in Extract 6 that interfere with the binding of the bioactive chemistries.

TABLE 9
Comparisons of the adhesion and post-binding adhesion of
C. albicans with cranberry Extract 5 and 6 are summarized.
Comparisons of Adhesion of C. albicans (ATCC#96133) using
the Adhesion and Post-binding Assays

| | Adhesion | Post-binding |
|---|---|---|
| | Assay | Assay |
| Extract/Extract Concentration | (% Inhibition) | (% Inhibition) |
| Extract 6/1000 μg mL⁻¹ | 62.06 | 65.08 |
| Extract 6/100 μg mL⁻¹ | 20.90 | 61.44 |
| Extract 5/1000 μg mL⁻¹ | 40.37 | 60.62 |
| Extract 5/100 μg mL⁻¹ | 21.40 | 35.65 |

TABLE 10
Comparisons of the adhesion and post-binding adhesion of E. coli
with cranberry Extract 6 are summarized.
Comparisons of Adhesion of E. coli (ATCC 53499) using
Adhesion and Post-binding Assays

| | Adhesion assay | Post-binding Assay |
|---|---|---|
| Extract/Extract Concentration | (% Inhibition) | (% Inhibition) |
| Extract 6/1000 μg mL⁻¹ | 44.52 | 49.65 |
| Extract 6/100 μg mL⁻¹ | −6.61 | 39.68 |

When S. aureus had bound bioactives from Extract 6, adhesion was inhibited (Table 11). At 1000 μg ml⁻¹ and 100 μg ml⁻¹ of the extract, the percent inhibition for adhesion after bioactives were bound (post-binding assay) decreased by 50% in the adhesion inhibition. This apparent loss of adhesion inhibition when bioactives are bound may result from the rapid growth of S. aureus in the post-binding assay, however, the mode-of-action of the bioactives remains the same.

TABLE 11
Summary of inhibition of adhesion of S. aureus by cranberry
Extract 6 when bioactives are bound and in response to the
whole extract.
Comparisons of Adhesion of S. aureus (MRSA ATCC#700787)
using Adhesion and Post-binding Assays

| | Adhesion assay | Post-binding Assay |
|---|---|---|
| Extract/Extract Concentration | (% Inhibition) | (% Inhibition) |
| Extract 6/1000 μg mL⁻¹ | 76.50 | 39.53 |
| Extract 6/100 μg mL⁻¹ | 57.73 | 28.94 |

F. Cranberry Extract Anti-adhesion and Biostatic Compounds

Cranberry Extract 5 contains 508 unique compounds, 94 of which were identified (see Table 5). From the 508 chemicals in the Extract, 5 known compounds were determined to be active inhibitors of C. albicans adhesion and/or growth (see Table 5). The same set of chemicals was identified in each analysis. This may be due to the impact of growth rate on adhesion. Table 12 lists the known compounds that were found to be active inhibitors of C. albicans adhesion and/or growth, along with their relative abundances.

Among the known compounds (see Table 5), aminolevulenic acid (terpenoid acid) and abscisic acid (carboxylic acid) would have biostatic activities as they are related to known growth inhibitor compounds, though these functions are not described in the literature. Fraxin, a hydroxycoumarin glycoside and S-petasine, an alkaloid, would both have strong microbial growth inhibition activities. Schisandrol B is a terpenol, and would be a strong inhibitor of cell division, and would therefore have biostatic activity.

TABLE 12
Bioactive compounds in cranberry Extract 5 that block adhesion
and impact growth are summarized along with their molecular mass,
chemical class, relative abundance, and weight per 100 mg dose.

| | | | Relative | |
|---|---|---|---|---|
| | Molecular | Chemical | Abundance | Wt per 100 mg |
| Compound | Mass | Class | (%) | (μg) |
| aminolevulinic | 131.152 | fatty acid | 16.9 | 1429.1 |
| acid | | | | |
| abscisic acid | 264.136 | sterolic acid | 15.4 | 1305.2 |
| S-petasine | 348.176 | alkaloid | 1.0 | 86.6 |
| fraxin | 370.090 | glucoside | 0.6 | 53.1 |
| schisandrol B | 416.184 | lignan | 1.3 | 108.3 |

TABLE 13
Summary of the bioactive compounds in cranberry Extract 6 that
possess anti-adhesion and growth inhibition activities. Included
are the molecular mass, chemical class, relative abundances,
and weight per 100 mg based on relative abundances.

| | | | Relative | Wt per |
|---|---|---|---|---|
| | Molecular | Chemical | Abundance | 100 mg |
| Compound Name | Mass | Class | (%) | (μg) |
| L-threonine | 119.058 | amino acid | 0.1 | 6 |
| aminolevulinic acid | 131.058 | amino acid | 0.7 | 62 |
| Cinnamaldehyde | 132.058 | terpene | 13.4 | 1249 |
| 4-hydroxybenzoic | 139.037 | phenolic acid | 0.9 | 86 |
| acid | | | | |
| anethole/ | 148.089 | terpene | 1.4 | 130 |
| cuminaldehyde | | | | |
| Chitosan | 149.069 | polysaccharide | 0.6 | 55 |
| α-phenylindol | 193.089 | aromatic | 2.5 | 232 |
| biotin | 244.088 | Vitamin | 1.3 | 120 |
| abscisic acid | 264.136 | sterolic acid | 2.3 | 214 |
| vestitol | 272.105 | flavanoid | 4.6 | 429 |
| S-petasine | 348.176 | alkaloid | 1.4 | 134 |
| fraxin | 370.090 | glucoside | 0.1 | 10 |
| schisandrol B | 416.184 | lignan | 1 | 95 |

TABLE 14
Compounds identified by DART-MS in Extract 6 that bind to
E. coli and block adhesion.

| | | |
|---|---|---|
| Compound Name | Measured Mass | Calculated Mass |

Compounds in Extract 6 that Bind to E. coli

| | | |
|---|---|---|
| L-threonine | 120.0554 | 120.0658 |
| cinnamaldehyde | 133.0544 | 133.0658 |
| 4-hydroxybenzoic acid | 139.0359 | 139.0395 |
| fraxin | 371.0855 | 371.0978 |

Compounds in Extract 6 that Bind to C. albicans

| | | |
|---|---|---|
| L-threonine | 120.0551 | 120.0658 |
| aminolevulinic acid | 132.0834 | 132.0658 |
| cinnamaldehyde | 133.0568 | 133.0658 |
| 4-hydroxybenzoic acid | 139.0356 | 139.0395 |
| anethole/cuminaldehyde | 149.1111 | 149.0968 |
| chitosan | 150.0555 | 150.0768 |
| biotin | 245.1029 | 245.0958 |
| abscisic acid | 265.1422 | 265.1438 |
| vestitol | 273.1328 | 273.1128 |

Compounds in Extract 6 that Bind to S. aureus (MRSA)

| | | |
|---|---|---|
| L-threonine | 120.0861 | 120.0658 |
| anethole/cuminaldehyde | 149.1184 | 149.0968 |
| chitosan | 150.0724 | 150.0768 |
| a-phenylindol | 194.0873 | 194.0968 |
| fraxin | 371.1064 | 371.0978 |

F. Pharmacokinetics

The anti-adhesion compounds in Extract 5 appeared in serum within 10 minutes from 5 healthy adults who ingested two vegcaps (300 mg dose) at time zero (FIG. 10). The key compounds included abscisic acid, aminolevulenic acid, fraxin, schisandrol B and S-petasine. The levels of the compounds increased through about 40 minutes and declined thereafter, though detectable levels persisted in serum through 1-2 hours. Interestingly, all of the compounds, except schisandrol B, were undetectable after 180 minutes, which showed a second peak of abundance at 240 minutes. Three of the compounds in Extract 5, abscisic acid, aminolevulenic acid, and S-petasine appeared in urine by the first 1-hr time point and persisted through the 8 hour sampling time (FIG. 11). Abscisic acid was the most abundant bioactive in the urine with levels reaching a peak at 2 hours (FIG. 11). The data show that the anti-adhesion compounds in Extract 5 appear in serum within minutes of ingestion.

The anti-adhesion and growth inhibition compounds in Extract 6 appeared in urine within the 1-hr time point from 5 healthy adults who ingested two vegcaps at time zero (FIG. 12). The key compounds included 4-hydroxybenzoic acid, abscisic acid, aminolevulenic acid, α-phenylindol, chitosan, cinnamaldehyde, L-threonine, S-petasine and vestitol. The levels of the compounds increased through 2 hours and declined thereafter. Interestingly, all of the compounds except aminolevulenic acid and S-petasine were still present at 8 hours post-ingestion of Extract 6 (FIG. 12). The data show that the key compounds in Extract 6 appear in urine rapidly and persist through 8 hours, the last time point evaluated in this study.

We claim:

1. A cranberry extract comprising at least one compound selected from the group consisting of 0.5 to 10% by weight aminoevulinic acid, 0.5 to 10% by weight of abscisic acid, 0.01 to 5% by weight of S-petasine, 0.01 to 5% by weight of fraxin, and 0.01 to 5% by weight of schisandrol B.

2. The cranberry extract of claim 1, wherein the extract comprises 0.01 to 5% by weight of schisandrol B.

3. The cranberry extract of claim 1, wherein the extract comprises 0.01 to 5% by weight of fraxin.

4. The cranberry extract of claim 1, wherein the extract comprises 0.01 to 5% by weight of S-petasine.

5. The cranberry extract of claim 1, wherein the extract comprises 0.5 to 10% by weight of abscisic acid.

6. The cranberry extract of claim 1, wherein the extract comprises 0.5 to 10% by weight aminoevulinic acid.

7. The cranberry extract of claim 1, wherein the extract comprises at least one compound selected from the group consisting of 0.5 to 5% by weight aminoevulinic acid, 0.5 to 5% by weight of abscisic acid, 0.01 to 2% by weight of S-petasine, 0.01 to 2% by weight of fraxin, and 0.05 to 3% by weight of schisandrol B.

8. A cranberry extract comprising at least one compound selected from the group consisting of 500 to 5000 μg aminoevulinic acid, 500 to 5000 μg abscisic acid, 10 to 1000 μg S-petasine, 5 to 1000 μg fraxin, and 10 to 1000 μg schisandrol B, per 100 mg of the extract.

9. A cranberry extract comprising cinnamaldehyde, 0.1 to 5% L-threonine by weight of the cinnamaldehyde, 1 to 10% aminoevulinic acid by weight of the cinnamaldehyde, 1 to 15% 4-hydroxybenzoic acid by weight of the cinnamaldehyde, 5 to 20% anethole/cuminaldehyde by weight of the cinnamaldehyde, 1 to 10% chitosan by weight of the cinnamaldehyde, 10 to 25% α-phenylindol by weight of the cinnamaldehyde, 5 to 20% biotin by weight of the cinnamaldehyde, 10 to 25% abscisic acid by weight of the cinnamaldehyde, 20 to 50% vestitol by weight of the cinnamaldehyde, 5 to 20% S-petasine by weight of the cinnamaldehyde, 0.1 to 5% fraxin by weight of the cinnamaldehyde, and 1 to 15% Schisandrol B by weight of the cinnamaldehyde.

10. A cranberry extract having a fraction comprising a Direct Analysis in Real Time (DART) mass spectrometry chromatogram of any of FIGS. 1 through 6.

11. A cranberry extract of claim 1, wherein the extract has an IC₅₀ value for C. albicans of less than 1000 μg/mL.

12. The cranberry extract of claim 11, wherein the IC₅₀ value for C. albicans is about 1 μg/mL to 500 μg/mL.

13. The cranberry extract of claim 11, wherein the IC₅₀ value for C. albicans is about 1 μg/mL to 50 μg/mL to 100 μg/mL.

14. The cranberry extract of claim 11, wherein the IC₅₀ value for C. albicans is about 1 μg/mL 50 μg/mL.

15. The cranberry extract of claim 1, wherein the IC₅₀ value for E. coli is less than 500 μg/mL

16. The cranberry extract of claim 15, wherein the IC₅₀ value for E. coli is about 0.05 to 100 μg/mL.

17. The cranberry extract of claim 15, wherein the IC₅₀ value for E. coli is about 0.05 to 50 μg/mL.

18. The cranberry extract of claim 1, wherein the IC₅₀ value for S. aureus is less than 3000 μg/mL.

19. The cranberry extract of claim 18, wherein the IC₅₀ value for S. aureus is less than 2000 μg/mL.

20. The cranberry extract of claim 18, wherein the IC₅₀ value for S. aureus is about 1 to 2000 μg/mL.

21. The cranberry extract of claim 18, wherein the IC₅₀ value for S. aureus is about 1 to 500 μg/mL.

22. The cranberry extract of claim 18, wherein the IC₅₀ value for S. aureus is about 1 to 250 μg/mL.

23. The cranberry extract of claim 18, wherein the IC₅₀ value for S. aureus is about 1 to 100 μg/mL.

24. A combined cranberry and cinnamon extract comprising at least one compound selected from 0.001 to 5% by weight L-threonine, 0.01 to 5% by weight aminoevulinic acid, 0.5 to 10% cinnamaldehyde, 0.01 to 5% by weight 4-hydroxybenzoic acid, 0.01 to 5% by weight anethole/cuminaldehyde, 0.01 to 5% by weight chitosan, 0.05 to 10% by weight a-phenylindol, 0.01 to 5% by weight biotin, 0.05 to 10% by weight abscisic acid, 0.1 to 10% by weight vestitol, 0.01 to 5% S-petasine, 0.001 to 5% by weight fraxin, and 0.01 to 5% by weight schisandrol B.

25. The extract of claim 24, comprising at least one compound selected from 0.001 to 2% by weight L-threonine, 0.01 to 2% by weight aminoevulinic acid, 0.5 to 5% cinnamaldehyde, 0.01 to 2% by weight 4-hydroxybenzoic acid, 0.01 to 2% by weight anethole/cuminaldehyde, 0.01 to 2% by weight chitosan, 0.05 to 5% by weight α-phenylindol, 0.01 to 2% by weight biotin, 0.05 to 5% by weight abscisic acid, 0.1 to 5% by weight vestitol, 0.01 to 2% S-petasine, 0.001 to 2% by weight fraxin, and 0.01 to 2% by weight schisandrol B.

26. A combined cranberry and cinnamon extract comprising at least one compound selected from the group consisting of 1 to 1000 μg L-threonine, 5 to 1000 μg aminoevulinic acid, 500 to 5000 μg cinnamaldehyde, 10 to 1000 μg 4-hydroxybenzoic acid, 10 to 1000 μg anethole/cuminaldehyde, 10 to 1000 μg chitosan, 50 to 1500 μg α-phenylindol, 10 to 1500 μg biotin, 50 to 1500 μg abscisic acid, 50 to 2000 μg vestitol, 10 to 1500 μg S-petasine, 1 to 1000 μg fraxin, and 10 to 1000 μg schisandrol B per 100 mg of extract.

27. A combined cranberry and cinnamon extract comprising 0.1 to 5% L-threonine by weight of the cinnamaldehyde, 1 to 10% aminoevulinic acid by weight of the cinnamaldehyde, 1 to 15% 4-hydroxybenzoic acid by weight of the cinnamaldehyde, 5 to 20% anethole/cuminaldehyde by weight of the cinnamaldehyde, 1 to 10% chitosan by weight of the cinnamaldehyde, 10 to 25% α-phenylindol by weight of the cinnamaldehyde, 5 to 20% biotin by weight of the cinnamaldehyde, 10 to 25% abscisic acid by weight of the cinnamaldehyde, 20 to 50% vestitol by weight of the cinnamaldehyde, 5 to 20% S-petasine by weight of the cinnamaldehyde, 0.1 to 5% fraxin by weight of the cinnamaldehyde, and 1 to 15% Schisandrol B by weight of the cinnamaldehyde.

28. A combined cranberry and cinnamon extract having a fraction comprising a Direct Analysis in Real Time (DART) mass spectrometry chromatogram of FIG. 6.

29. A combined cranberry and cinnamon extract of claim 1, wherein the extract has an IC₅₀ value for C. albicans of less than 1000 μg/mL.

30. The combined cranberry and cinnamon extract of claim 29, wherein the IC₅₀ value for C. albicans is about 1 μg/mL to 50 μg/mL.

31. The combined cranberry and cinnamon extract of claim 30, wherein the IC₅₀ value for C. albicans is about 1 μg/mL to 50 μg/mL to 100 μg/mL.

32. The combined cranberry and cinnamon extract of claim 30, wherein the IC₅₀ value for C. albicans is about 1 μg/mL 50 μg/mL.

33. The combined cranberry and cinnamon extract of claim 1, wherein the IC₅₀ value for E. coli is less than 500 μg/mL

34. The combined cranberry and cinnamon extract of claim 33, wherein the IC₅₀ value for E. coli is about 0.05 to 100 μg/mL.

35. The combined cranberry and cinnamon extract of claim 33, wherein the IC₅₀ value for E. coli is about 0.05 to 50 μg/mL.

36. The combined cranberry and cinnamon extract of claim 1, wherein the IC₅₀ value for S. aureus is less than 3000 μg/mL

37. The combined cranberry and cinnamon extract of claim 36, wherein the IC₅₀ value for S. aureus is less than 2000 μg/mL.

38. The combined cranberry and cinnamon extract of claim 36, wherein the IC₅₀ value for S. aureus is about 1 to 2000 μg/mL.

39. The combined cranberry and cinnamon extract of claim 36, wherein the IC₅₀ value for S. aureus is about 1 to 500 μg/mL.

40. The cranberry extract of claim 36, wherein the IC₅₀ value for S. aureus is about 1 to 250 μg/mL.

41. The cranberry extract of claim 36, wherein the IC₅₀ value for S. aureus is about 1 to 100 μg/mL.

42. A pharmaceutical composition comprising a cranberry extract of any one of claim 1 and a pharmaceutically acceptable carrier.

43. A method of treating or preventing an infection, comprising administering to a subject in need thereof a therapeutically effective amount of the composition of claim 42.

44. The method of claim 43, wherein the infection is a bacterial infection or a fungal infection.

45. The method of claim 43, wherein the infection is selected from the group consisting of C. albicans, E. coli, or S. aureus.

46. The method of claim 43, wherein the infection is a yeast infection.

47. The method of claim 43, wherein the infection is a Staphylococcus infection.

48. The method of claim 43, wherein the infection is a methicillin resistant (MRSA) Staphylococcus infection.

49. The method of claim 43, wherein the infection is a urinary tract infection.

50. The composition of claim 42, wherein the composition is formulated as a suppository for vaginal administration.

51. The composition of claim 42, wherein the composition is formulated as a lotion, cream, ointment, oil, paste or transdermal patch and the administration is topical.

52. The composition of claim 42, wherein the composition is formulated as a functional food, dietary supplement, powder or beverage.

53. A cranberry extract prepared by a process comprising:
a) providing a cranberry feedstock; and
b) extracting the cranberry feedstock with dimethylsulfoxide; and
c) isolating the extract.

54. The extract of claim 53, wherein in the process further comprises
d) providing a second cranberry feedstock
e) extracting the second feedstock with aqueous ethanol to form an aqueous ethanol extract;
f) separating the aqueous Ethanolic extract on a chromatography column with aqueous methanol;
g) collecting a 100% methanol fraction from the separation;
h) combining the methanol fraction of step g) with the extract of step c).
